(19) United States
(12) Patent Application Publication
(10) Pub. No.: US 2024/0408207 A1
(43) Pub. Date: Dec. 12, 2024

(54) COMPOSITIONS AND METHODS FOR TREATING CANCER BY TARGETING ENDOTHELIAL CELLS HAVING UPREGULATED EXPRESSION OF TRANSMEMBRANE MOLECULES

(71) Applicant: President and Fellows of Harvard College, Cambridge, MA (US)

(72) Inventors: Munir M. Mosaheb, Cambridge, MA (US); Ulrich H. Von Andrian, Canbridge, MA (US)

(73) Assignee: President and Fellows of Harvard College, Cambridge, MA (US)

(21) Appl. No.: 18/668,547

(22) Filed: May 20, 2024

Related U.S. Application Data
(63) Continuation of application No. PCT/US2022/080449, filed on Nov. 23, 2022.
(60) Provisional application No. 63/282,565, filed on Nov. 23, 2021.

(51) Int. Cl. A61K 39/00; C07K 14/725; C07K 16/30

(52) U.S. Cl. CPC A61K 39/464493; A61K 39/4611; A61K 39/4631; C07K 14/7051; C07K 16/3069; C07K 2317/569; C07K 2317/732

(57) ABSTRACT

The technology described herein is directed to targeting molecules that bind to one or more transmembrane molecules expressed in tumor vascular endothelial cells and that are capable of targeting an agent that induces cell death, for example, immunogenic or non-immunogenic cancer cell death. In another aspect, described herein are methods of treating a subject having cancer comprising administering said targeting molecules and agents, and, in certain embodiments, enhancing the immunogenic response to the cancer, for example, by enhancing intratumoral infiltration of T cells.

### RELATED APPLICATIONS

This instant application is a continuation of International Application No. PCT/US2022/080449, filed Nov. 23, 2022, which claims the benefit of priority to U.S. Provisional Application No. 63/282,565, filed on Nov. 23, 2021. The entire contents of each of the foregoing applications are expressly incorporated herein by reference.

### GOVERNMENT SUPPORT

This invention was made with government support under AR068383 and AI112521 awarded by the National Institutes of Health. The government has certain rights to this invention.

### REFERENCE TO ELECTRONIC SEQUENCE LISTING

The application contains a Sequence Listing which has been submitted electronically in .XML format and is hereby incorporated by reference in its entirety. Said .XML copy, created on Jun. 27, 2024, is named “117823-30903.xml” and is 24,680 bytes in size. The sequence listing contained in this .XML file is part of the specification and is hereby incorporated by reference herein in its entirety.

### TECHNICAL FIELD

Described herein are compositions and methods related to targeting and treating cancer with targeting molecules that bind upregulated transmembrane molecules in tumor vascular endothelial cells.

### BACKGROUND OF THE INVENTION

Recent clinical experience indicates that a cancer patient's immune system can be therapeutically harnessed to attack malignant tumors and induce long-lasting tumor regression (1). For example, treatment with anti-CTLA-4, an immune checkpoint inhibitor (CPI), results in tumor regression and long-term survival of a subset of patients (˜20%) with certain types of cancers, such as melanoma (2). Similarly, anti-PD1 antibodies have also been successful for some patients and are used as first-line therapy for melanoma, gastric cancer, hepatocellular carcinoma, head and neck squamous cell carcinoma and urothelial cancer (3).

However, despite this progress, current immunotherapy regimens show efficacy only in a subset of malignancies and/or a minority of patients. The high failure rate of cancer immunotherapy is inversely correlated with the presence of tumor-infiltrating T lymphocytes (TILs). It has been documented in many studies that many immunotherapy resistant tumors lack T cells within the tumors and these cells instead accumulate in the tumor periphery, also a common observation by clinical pathologist (4-6).

The latest advances in cancer-immunotherapy have provided patients with a new type of treatment in which chimeric antigen receptor (CAR) T cells are generated to target malignant cells. However, CAR T cells have only limited clinical success for solid tumors (10, 11). This is probably due to the fact that blood-borne T cells are often unable to overcome the vascular barrier posed by the local microcirculation to access extravascular tumor cells. Moreover, even in settings where tumors are successfully targeted, the inherent genetic instability may allow tumor cells to acquire mutations resulting in resistance. Many tumors are also highly heterogeneous among and even within patients, so the efficacy of direct tumor targeting strategies can be highly variable.

The reason(s) for the paucity of T cells in so-called non-inflammatory tumors (which have a poor prognosis) are not well understood, but likely involve the inability of circulating tumor specific T cells to adhere to and emigrate from local microvessels into the surrounding tumor.

### SUMMARY OF THE INVENTION

A cancer patient's immune system can be therapeutically harnessed to eliminate malignant tumors. However, current immunotherapy regimens have shown efficacy only in a minority of malignancies. This high failure rate is inversely correlated with the presence of tumor-infiltrating T cells. The reasons for the paucity of T cells in so-called non-inflammatory (immunotherapy-resistant) tumors are poorly understood, but likely involve the inability of circulating T cells to adhere to and emigrate from tumor microvessels into surrounding tissue. The present invention involves unique transmembrane molecules (e.g., proteins) that are upregulated in both murine and human tumor microvasculature and not in healthy tissues. These differentially-expressed transmembrane molecules allow for tumor targeted treatment, via a targeting molecule, such as targeted CAR T cell therapy and other modes of targeted delivery of therapeutics. In another aspect, the present targeting molecules enable highly specific diagnostic imaging.

Specifically, the identification of these upregulated transmembrane molecules allows for generation of targeting molecules against these tumor-restricted endothelial markers to selectively target tumor microvessels in immunotherapy-resistant tumors. Accordingly, in one aspect, the invention comprises a platform approach for targeting the intra-humoral microvasculature, which allows for the present inventive compositions and methods to increase T cell recruitment into tumors so as to boost endogenous anti-tumor immunity and to synergize with other immuno-oncology approaches.

Accordingly, in one aspect, the present invention provides a targeting molecule, wherein the targeting molecule binds to a transmembrane molecule on a tumor cell in which expression of the transmembrane molecule is upregulated and wherein the transmembrane molecule is selected from the group consisting of molecules set forth in Tables 8-10. Optionally, the tumor cell is a tumor vascular endothelial cell or a tumor stroma cell.

In some embodiments, the tumor vascular endothelial cell is a venular cell.

In some embodiments, the transmembrane molecule is not expressed in non-tumor cells, the transmembrane molecule is expressed at higher levels in the tumor cells as compared to in non-tumor cell cells, or the transmembrane molecule is a variant of a transmembrane protein expressed in non-tumor vascular endothelial cells.

In some embodiments, the transmembrane molecule is not expressed in non-tumor vascular endothelial cells or non-tumor stroma cells, the transmembrane molecule is expressed at higher levels in the tumor vascular endothelial cells or the tumor stroma cells as compared to in non-tumor vascular endothelial cell or non-tumor stroma cells, or the transmembrane molecule is a variant of a transmembrane protein expressed in non-tumor vascular endothelial cells or non-tumor stroma cells.

In some embodiments, the transmembrane molecule is expressed at least 1.5-fold, at least 2-fold, at least 2.5-fold, at least 3-fold, at least 3.5-fold, at least 4-fold, at least 4.5-fold, or at least 5-fold more in the tumor vascular endothelial cells or the tumor stromal cells as compared to expression in non-tumor vascular endothelial cells or non-tumor stromal cells. In some embodiments, the expression of the transmembrane molecule is upregulated as compared to a control level. In some embodiments, the control level is the level of expression of the transmembrane molecule in a non-tumor vascular endothelial cell or non-tumor stromal cell.

In some embodiments, the targeting molecule is an antibody or antigen-binding fragment thereof. In some embodiments, the antibody or antigen-binding fragment is a monoclonal antibody, human antibody, a humanized antibody, a chimeric antibody, a recombinant antibody, a multispecific antibody, or an antigen-binding fragment thereof; wherein the antigen-binding fragment is 1) an Fv, Fab, F(ab′)2, Fab′, dsFv, scFv, or sc (Fv)2; 2) a diabody, ScFv, SMIP, single chain antibody, affibody, avimer, or nanobody; or 3) a single domain antibody. In some embodiments, the antigen-binding fragment is a nanobody.

In another aspect, the present invention provides a composition comprising 1) the targeting molecule of various embodiments of the above aspects or any other aspect in the invention delineated herein, and 2) an agent that (a) induces cell death to a tumor cell in which the expression of at least one transmembrane molecule selected from the group consisting of those molecules set forth in Tables 8-10 is upregulated as compared to a non-tumor vascular endothelial control cell, or (b) induces an inflammatory response. In certain embodiments, the tumor cell is a tumor vascular endothelial cell or a tumor stromal cell.

In certain embodiments, the agent that induces cell death is an agent that induces immunogenic cell death. Alternatively, in other embodiments, the agent that induces cell death is an agent that induces non-immunogenic cell death.

In some embodiments, the agent is selected from the group consisting of a small molecule, saccharide, oligosaccharide, polysaccharide, peptide, protein, peptide analog and derivatives, peptidomimetic, siRNAs, shRNAs, antisense RNAs, ribozymes, dendrimers, aptamers, and any combination thereof.

In some embodiments, the agent that induces an inflammatory response is a TLR4 agonist or GP-130 agonist. In some embodiments, the agent that induces cell death is a chemotherapeutic agent. In some embodiments, the agent that induces cell death is an engineered CAR-immune cell, optionally the CAR-immune cell is a CAR-T cell, CAR-macrophages, CAR-monocyte, CAR-granulocyte, CAR-NK cell, a CAR-NKT cell, a tumor infiltrating lymphocyte (TIL), a cell expressing an antigen recognizing a tumor antigen or a cell expressing a receptor recognizing an antibody bound to the surface of a tumor cell. In some embodiments, the agent is coupled to or is co-administered with the targeting molecule of the invention. In another aspect, the present invention provides a pharmaceutical composition comprising 1) the targeting molecule of various embodiments of the above aspects or any other aspect in the invention delineated herein, or the composition of various embodiments of the above aspects or any other aspect in the invention delineated herein, and 2) a pharmaceutically acceptable carrier.

In some embodiments, the pharmaceutical composition comprises a lipid formulation. In some embodiments, the lipid formulation comprises a lipid nanoparticle.

In another aspect, the present invention provides a method of treating cancer in a subject in need thereof, comprising administering any of the compositions disclosed herein or administering any of the pharmaceutical compositions disclosed herein. In addition, the present invention provides composition for treating cancer in a subject, wherein the composition comprises any of the compositions disclosed herein or any of the pharmaceutical compositions disclosed herein. Further, the present invention provides for use of any of the compositions disclosed herein or any of the pharmaceutical compositions disclosed herein for the preparation of a medicament for the treatment of cancer.

In another aspect, the present invention provides a method of treating cancer in a subject in need thereof, wherein the cancer is characterized by a tumor cell, such as a tumor vascular endothelial cell or tumor stromal cell, in which the expression of at least one transmembrane molecule is upregulated, comprising administering to the subject a composition comprising a targeting molecule which binds to the transmembrane molecule on the tumor cell and an agent that induces cancer cell death, optionally wherein the composition is a composition of various embodiments of the above aspects or any other aspect in the invention delineated herein, or the pharmaceutical composition of various embodiments of the above aspects or any other aspect in the invention delineated herein. For example, in any of these methods, the transmembrane molecule is selected from the group consisting of the molecules set forth in Tables 8-10.

In another aspect, the present invention provides a composition for treating cancer in a subject in need thereof, wherein the cancer is characterized by a tumor cell, such as a tumor vascular endothelial cell or a tumor stromal cell, in which the expression of at least one transmembrane molecule is upregulated, wherein the composition comprises a targeting molecule which binds to the transmembrane molecule on the tumor cell and an agent that induces cancer cell death, optionally wherein the composition is a composition of various embodiments of the above aspects or any other aspect in the invention delineated herein, or the pharmaceutical composition of various embodiments of the above aspects or any other aspect in the invention delineated herein. For example, in any of the compositions for treating cancer, the composition comprises a transmembrane molecule selected from the group consisting of the molecules set forth in Tables 8-10.

In another aspect, the present invention provides for use of a composition for the preparation of a medicament for treating cancer in a subject in need thereof, wherein the cancer is characterized by a tumor cell, such as a tumor vascular endothelial cell or a tumor stromal cell, in which the expression of at least one transmembrane molecule is upregulated, wherein the composition comprises a targeting molecule which binds to the transmembrane molecule on the tumor cell and an agent that induces cancer cell death, optionally wherein the composition is a composition of various embodiments of the above aspects or any other aspect in the invention delineated herein, or the pharmaceutical composition of various embodiments of the above aspects or any other aspect in the invention delineated herein. For example, in any of the uses, the composition comprises a transmembrane molecule selected from the group consisting of the molecules set forth in Tables 8-10.

In another aspect, the present invention provides a method of treating cancer in a subject in need thereof, comprising administering to the subject the composition of various embodiments of the above aspects or any other aspect in the invention delineated herein, or the pharmaceutical composition of various embodiments of the above aspects or any other aspect in the invention delineated herein.

In addition, the present invention provides compositions for treating cancer in a subject in need thereof, wherein the composition comprises various embodiments of the above aspects or any other aspect in the invention delineated herein, or the pharmaceutical composition of various embodiments of the above aspects or any other aspect in the invention delineated herein.

The invention also provides for use of the various embodiments of the above aspects or any other aspect in the invention delineated herein, or the pharmaceutical composition of various embodiments of the above aspects or any other aspect in the invention delineated herein for the preparation of a medicament for the treatment of cancer.

In some embodiments, the agent is coupled to or is co-administered with the targeting molecule. In some embodiments, the agent is co-administered with a lipid nanoparticle comprising the targeting molecule.

In some embodiments, the expression of the transmembrane molecule is upregulated as compared to a control level.

In some embodiments, the control level is the level of expression of the transmembrane molecule in a non-tumor cell, such as a non-tumor vascular endothelial cell or a non-tumor stromal cell.

In some embodiments, the method further comprising identifying in the subject the presence of the tumor cell, such as a tumor vascular endothelial cell or a tumor stromal cell, in which the expression of the at least one transmembrane molecule is upregulated as compared to expression of the transmembrane molecule in a non-tumor cell and wherein the transmembrane molecule is selected from the group consisting of molecules set forth in Tables 8-10.

In some embodiments, the composition for treating cancer or the medicament is administered to a subject identified to have the presence of a tumor cell, such as a tumor vascular endothelial cell or a tumor stromal cell, in which the expression of the at least one transmembrane molecule is upregulated as compared to expression of the transmembrane molecule in a non-tumor cell and wherein the transmembrane molecule is selected from the group consisting of molecules set forth in Tables 8-10.

In some embodiments, the method elicits or enhances an immune response to the cancer. In some embodiments, the method increases the level or activity of intra-tumoral T cells. In some embodiments, the level or activity of intra-tumoral T cells are increased at least 1.5-fold, at least 2-fold, at least 2.5-fold, at least 3-fold, at least 3.5-fold, at least 4-fold, at least 4.5-fold, or at least 5-fold more after administration as compared to the level or activity of intra-tumoral T cells prior to administration.

In some embodiments, administration of any of the compositions for treating cancer or administration of any of the medicaments described above elicits or enhances an immune response to the cancer. In some embodiments, administration increases the level or activity of intra-tumoral T cells. In some embodiments, the level or activity of intra-tumoral T cells are increased at least 1.5-fold, at least 2-fold, at least 2.5-fold, at least 3-fold, at least 3.5-fold, at least 4-fold, at least 4.5-fold, or at least 5-fold more after administration as compared to the level or activity of intra-tumoral T cells prior to administration.

In another aspect, the present invention provides a method of treating cancer in a subject in need thereof, comprising modifying the expression in a tumor cell, such as a tumor vascular endothelial cell or a tumor stromal cell, of at least one transmembrane molecule which is upregulated, optionally wherein the transmembrane molecule is selected from the group consisting of those molecules set forth in Tables 8-10, wherein modifying the expression of at least one transmembrane molecule comprises delivering a nucleic acid capable of modifying gene expression of the at least one transmembrane molecule.

In another aspect, the present invention provides a composition for treating cancer in a subject in need thereof, wherein the composition comprises a nucleic acid capable of modifying gene expression of at least one transmembrane molecule and administration of the composition modifies the expression in a tumor cell, such as a tumor vascular endothelial cell or a tumor stromal cell, of at least one transmembrane molecule which is upregulated, optionally wherein the transmembrane molecule is selected from the group consisting of those transmembrane molecules set forth in Tables 8-10.

In another aspect, the present invention provides a nucleic acid for the preparation of a medicament for treating cancer in a subject in need thereof, wherein the nucleic acid is capable of modifying gene expression of at least one transmembrane molecule and administration of the medicament modifies the expression in a tumor cell, such as a tumor vascular endothelial cell or a tumor stromal cell, of at least one transmembrane molecule which is upregulated, optionally wherein the transmembrane molecule is selected from the group consisting of those transmembrane molecules set forth in Tables 8-10.

In some embodiments, the method further comprising identifying in the subject the presence of the tumor cell, such as a tumor vascular endothelial cell or a tumor stromal cell, in which the expression of the at least one transmembrane molecule is upregulated as compared to a control level. In some embodiments, the composition for treating cancer or the medicament for treating cancer is administered to a subject identified as having the presence of the tumor cell, such as a tumor vascular endothelial cell or a tumor stromal cell, in which the expression of the at least one transmembrane molecule is upregulated as compared to a control level.

In some embodiments, the control level is the level of expression of the transmembrane molecule in a non-tumor control cell, such as a non-tumor vascular endothelial control cell or a non-tumor stromal control cell.

In some embodiments, the nucleic acid is selected from the group consisting of an antisense RNA, siRNA, shRNA, and a CRISPR system. In some embodiments, the nucleic acid encodes a CRISPR system.

In some embodiments, the CRISPR system comprises i) one or more guide RNAs (gRNAs), wherein the gRNA targets at least one transmembrane molecule gene or promoter region; and ii) a Cas9 protein, wherein the Cas9 protein is nuclease deficient (dCas9). In some embodiments, the dCas9 protein further comprises an effector molecule. In some embodiments, the effector molecule is selected from the group consisting of DNA-binding domain, epigenetic modifier, and a nuclease. In some embodiments, the DNA-binding domain is a DNA-binding domain from a Transcription activator-like effector (TALE) polypeptide or a zinc finger (ZNF) polypeptide. In some embodiments, the epigenetic modifier is selected form the group consisting of a DNA methyltransferase, histone acetyltransferase, histone deacetylase, histone methyltransferase, and histone demethylase.

In some embodiments, the nucleic acid encodes the antisense RNA, siRNA, or shRNA, wherein the antisense RNA, siRNA, or shRNA targets an mRNA of at least one transmembrane molecule.

In some embodiments, the nucleic acid is present in a vector, wherein the vector is a viral expression vector. In some embodiments, the viral expression vector is present in a composition. In some embodiments, the composition comprises a pharmaceutical composition. In some embodiments, the pharmaceutical composition comprises a lipid formulation.

In some embodiments, the lipid formulation comprises the targeting molecule of various embodiments of the above aspects or any other aspect in the invention delineated herein.

In some embodiments, the targeting molecule binds the same transmembrane molecule that the nucleic acid is capable of modifying expression of. In some embodiments, the targeting molecule binds a different transmembrane molecule that the nucleic acid is capable of modifying expression of.

In some embodiments, the at least one transmembrane molecule is not expressed in non-tumor cells, such as a non-tumor vascular endothelial cells or a non-tumor stromal cell, the at least one transmembrane molecule is expressed at higher levels in the tumor cells, such as a tumor vascular endothelial cells or tumor stromal cells, as compared to in non-tumor cells, or the transmembrane molecule is a variant of a transmembrane protein expressed in non-tumor cell.

In some embodiments, the method further comprising determining that expression of the at least one transmembrane molecule has been decreased as compared to a control cell that has not been administered the nucleic acid. In some embodiments, the method further comprising determining that expression of at least one transmembrane molecule has been increased as compared to a control cell that has not been administered the nucleic acid.

In some embodiments, the composition for treating cancer or the medicament for treating cancer is administered to a subject determined to have expression of at least one transmembrane molecule decreased as compared to a control cell that has not been administered the nucleic acid. In some embodiments, the composition for treating cancer or the medicament for treating cancer is administered to a subject determined to have expression of at least one transmembrane molecule increased as compared to a control cell that has not been administered the nucleic acid.

In another aspect, the present invention provides a method of treating cancer in a subject in need thereof, comprising administering to a subject having cancer an immune effector cell expressing a chimeric antigen receptor (CAR), wherein the CAR comprises the targeting molecule of various embodiments of the above aspects or any other aspect in the invention delineated herein, wherein the targeting molecule binds to a transmembrane molecule on a tumor cell, such as a tumor vascular endothelial cell or a tumor stromal cell, in which expression of the transmembrane molecule is upregulated. For example, in any of these methods, the transmembrane molecule is selected from the group consisting of molecules set forth in Tables 8-10.

In another aspect, the present invention provides a composition for treating cancer in a subject in need thereof, wherein the composition comprises an immune effector cell expressing a chimeric antigen receptor (CAR), wherein the CAR comprises the targeting molecule of various embodiments of the above aspects or any other aspect in the invention delineated herein, wherein the targeting molecule binds to a transmembrane molecule on a tumor cell, such as a tumor vascular endothelial cell or a tumor stromal cell, in which expression of the transmembrane molecule is upregulated. For example, in any of the compositions, the transmembrane molecule is selected from the group consisting of molecules set forth in Tables 8-10.

In another aspect, the present invention provides use of a composition for treating cancer for the preparation of a medicament for treating cancer in a subject in need thereof, wherein the composition comprises an immune effector cell expressing a chimeric antigen receptor (CAR), wherein the CAR comprises the targeting molecule of various embodiments of the above aspects or any other aspect in the invention delineated herein, wherein the targeting molecule binds to a transmembrane molecule on a tumor cell, such as a tumor vascular endothelial cell or a tumor stromal cell, in which expression of the transmembrane molecule is upregulated. For example, in any of these uses, the transmembrane molecule is selected from the group consisting of molecules set forth in Tables 8-10.

In another embodiment, the invention provides for a method of treating cancer in a subject in need thereof, comprising administering to a subject having cancer an immune effector cell expressing a chimeric antigen receptor (CAR), wherein a targeting molecule of various embodiments of the above aspects or any other aspect in the invention delineated herein, is expressed on the cell surface of the immune effector cell, wherein the targeting molecule binds to a transmembrane molecule on a tumor vascular endothelial cell in which expression of the transmembrane molecule is upregulated. In any of these methods, the immuno effector cell is a T-cell, macrophage, monocyte, granulocyte, natural killer (NK) cell or a natural killer T-cell (NKT-cell).

In another aspect, the present invention provides a composition for treating cancer in a subject in need thereof, wherein the composition comprises an immune effector cell expressing a chimeric antigen receptor (CAR), wherein a targeting molecule of various embodiments of the above aspects or any other aspect in the invention delineated herein, wherein the targeting molecule binds to a transmembrane molecule on a on a tumor cell, such as a tumor vascular endothelial cell or a tumor stromal cell, in which expression of the transmembrane molecule is upregulated. For example, in any of the compositions, the transmembrane molecule is selected from the group consisting of molecules set forth in Tables 8-10. In any of these compositions, the immuno effector cell is a T-cell, macrophages, natural killer (NK) cell or a natural killer T-cell (NKT-cell). In another aspect, the present invention provides use of a composition for the preparation of a medicament for treating cancer in a subject in need thereof, wherein the composition comprises an immune effector cell expressing a chimeric antigen receptor (CAR), wherein a targeting molecule of various embodiments of the above aspects or any other aspect in the invention delineated herein, wherein the targeting molecule binds to a transmembrane molecule on a on a tumor cell, such as a tumor vascular endothelial cell or a tumor stromal cell, in which expression of the transmembrane molecule is upregulated. For example, in any of the compositions, the transmembrane molecule is selected from the group consisting of molecules set forth in Tables 8-10. In any of these methods, the immuno effector cell is a T-cell, macrophage, natural killer (NK) cell or a natural killer T-cell (NKT-cell). In some embodiments, the method further comprising identifying in the subject the presence of the tumor cell, such as a tumor vascular endothelial cell or a tumor stromal cell, in which the expression of the at least one molecule is upregulated as compared to in a non-tumor cell, such as a non-tumor vascular endothelial cell or a non-tumor stromal cell.

In some embodiments, the composition for treating cancer or the medicament for treating cancer is administered to a subject identified as having the presence of the tumor cell, such as a tumor vascular endothelial cell or a tumor stromal cell, in which the expression of the at least one transmembrane molecule is upregulated as compared to a control level.

In some embodiments, the method or administration of the composition for treating cancer or administration of the medicament for treating cancer elicits or enhances an immune response to the cancer, optionally by increasing the level or activity of intra-tumoral T cells. In some embodiments, the level or activity of intra-tumoral T cells is increased at least 1.5-fold, at least 2-fold, at least 2.5-fold, at least 3-fold, at least 3.5-fold, at least 4-fold, at least 4.5-fold, or at least 5-fold more after administration as compared to the level or activity of intratumoral T cells to prior administration.

In some embodiments, the method or administration of the composition for treating cancer or administration of the medicament for treating cancer elicits an inflammatory response.

In another aspect, the present invention provides a method of diagnosing or prognosing cancer in a subject, comprising determining the expression of at least one transmembrane molecule selected from the group consisting of those molecules set forth in Tables 8-10 on a tumor cell, such as a tumor vascular endothelial cell or tumor stromal cell, wherein upregulation of expression of the at least one molecule on the tumor cell as compared to a control level is indicative of the presence or progression of the cancer.

In some embodiments, the control level is the level of expression of the transmembrane molecule in a non-tumor cell, such as a non-tumor vascular endothelial cell or a non-tumor stromal cell.

In some embodiments, the method further comprises a step of administering an agent that induces cancer cell death, optionally wherein the agent is any of the compositions described herein or any of the pharmaceutical compositions described herein. In another aspect, the present invention provides a method of determining the efficacy of treatment of cancer in a subject, comprising i) determining the expression of at least one transmembrane molecule selected from the group consisting of those molecules set forth in Tables 8-10 on a tumor cell, such as a tumor vascular endothelial cell or a tumor stromal cell, prior to administering a cancer treatment, wherein increased expression of the at least one transmembrane molecule on the tumor cell as compared to a control level is indicative of the presence or progression of the cancer; ii) determining the expression of the at least one transmembrane molecule after administration of the cancer treatment, wherein decreased expression of the at least one transmembrane molecule as compared to a control level is indicative of effective cancer treatment.

In some embodiments, the control level is the expression of the transmembrane molecule on a tumor cell, such as a tumor vascular endothelial cell or a tumor stromal cell, prior to administering the cancer treatment. In some embodiments, the tumor cell is a tumor vascular endothelial cell, and in some embodiments the tumor vascular endothelial cell is a venular cell.

In some embodiments, the transmembrane molecule is not expressed in non-tumor cells, such as non-tumor vascular endothelial cells or non-tumor stromal cells, the molecule is expressed at higher levels in the tumor cells as compared to non-tumor cells, or the transmembrane molecule is a variant of a transmembrane protein expressed in a non-tumor cell.

In some embodiments, the transmembrane molecule is expressed at least 1.5-fold, at least 2-fold, at least 2.5-fold, at least 3-fold, at least 3.5-fold, at least 4-fold, at least 4.5-fold, or at least 5-fold more in tumor cells, such as tumor vascular endothelial cells or tumor stromal cells, as compared to in non-tumor cells.

In some embodiments, the cancer is (i) a non-immunogenic cancer; (ii) a hematological cancer; or (iii) a solid tumor.

In some embodiments, the cancer is selected from the group consisting of melanoma, pancreatic cancer, and colorectal cancer.

In some embodiments, the cancer is breast cancer, prostate cancer, renal cell carcinoma, bone metastasis, lung cancer or metastasis, osteosarcoma, multiple myeloma, astrocytoma, pilocytic astrocytoma, dysembryoplastic neuroepithelial tumor, oligodendrogliomas, ependymoma, glioblastoma multiforme, mixed gliomas, oligoastrocytomas, medulloblastoma, retinoblastoma, neuroblastoma, germinoma, teratoma, gangliogliomas, gangliocytoma, central gangliocytoma, primitive neuroectodermal tumors (PNET, e.g. medulloblastoma, medulloepithelioma, neuroblastoma, retinoblastoma, ependymoblastoma), tumors of the pineal parenchyma (e.g. pineocytoma, pineoblastoma), ependymal cell tumors, choroid plexus tumors, neuroepithelial tumors of uncertain origin (e.g. gliomatosis cerebri, astroblastoma), esophageal cancer, colorectal cancer, CNS, ovarian, melanoma pancreatic cancer, squamous cell carcinoma, hematologic cancer (e.g., leukemia, lymphoma, and multiple myeloma), colon cancer, rectum cancer, stomach cancer, kidney cancer, pancreas cancer, skin cancer, or a combination thereof.

In any of the methods, uses or compositions for use disclosed herein, the tumor cell, such as a tumor vascular endothelial cell or a tumor stromal cell, has modified expression, e.g., upregulated expression or downregulated expression, of at least 1, or least 2, or at least 3, or at least 4, or at least 5, or least 6, or at least 7, or at least 8, or at least 9 or at least 10 transmembrane molecules set forth in Tables 8-10.

For example, the tumor cell, such as a tumor vascular endothelial cell or a tumor stromal cell, has upregulated expression of at least 1, or least 2, or at least 3, or at least 4, or at least 5, or least 6, or at least 7, or at least 8, or at least 9 or at least 10 transmembrane molecules set forth in Table 8, and particularly from the set of transmembrane molecules set forth in Table 8A, or Table 8B, or Table 8C, or Table 8D, or Table 8E, or Table 8F, and wherein the tumor cell is in a melanoma, pancreatic tumor, or colorectal tumor.

For example, the tumor cell, such as a tumor vascular endothelial cell or a tumor stromal cell, has upregulated expression of at least 1, or least 2, or at least 3, or at least 4, or at least 5, or least 6, or at least 7, or at least 8, or at least 9 or at least 10 transmembrane molecules set forth in Table 9, and particularly from the set of transmembrane molecules set forth in Table 9A, or Table 9B, or Table 9C, or Table 9D, or Table 9E, wherein the tumor cell is in a melanoma, pancreatic tumor, or colorectal tumor.

For example, the tumor cell, such as a tumor vascular endothelial cell or a tumor stromal cell, has upregulated expression of at least 1, or least 2, or at least 3, or at least 4, or at least 5, or least 6, or at least 7, or at least 8, or at least 9 or at least 10 transmembrane molecules set forth in Table 10, and particularly from the set of transmembrane molecules Table 10A, or Table 10B, or Table 10C, or Table 10D, or Table 10E, or Table 10F, wherein the tumor cell is in a melanoma, pancreatic tumor, or colorectal tumor.

### BRIEF DESCRIPTION OF THE DRAWINGS

FIGS. 1A-1D. FIG. 1A depicts imaging shows the proximity between VEC and T cells in MC38. Scale bar: 50 μm. FIG. 1B depicts gating strategy for EC and T cell. FIG. 1C depicts selection of CD31+ and/or DARC+ cells from tumors, peritumoral and healthy tissues from mice with MC38 tumors. FIG. 1D depicts selection of CD31+ and/or DARC+ cells from tumors, peritumoral and healthy tissues from mice with B16F10 tumors.

FIGS. 2A-2G. FIG. 2A depicts number of VEC per gram of tissue in MC38 and B16 subcutaneous (SubQ) tumors, peritumoral tissues and healthy skin. FIG. 2B depicts number of CD8+ T cells per gram of tissue in MC38 and B16 subcutaneous (SubQ) tumors, peritumoral tissues and healthy skin. FIG. 2C depicts correlation between the number of VEC and the number of CD8+ T cells in MC38 tumor models in mice. FIG. 2D depicts correlation between the number of VEC and the number of CD8+ T cells in B16 tumor models in mice. FIG. 2E depicts percentage of CD31+DARC+ blood endothelial cells in MC38 and B16 subcutaneous (SubQ) tumors, peritumoral tissues and healthy skin. FIG. 2F depicts percentage of CD3+CD8+ T cells among all CD45+ cells in MC38 and B16 subcutaneous (SubQ) tumors, peritumoral tissues and healthy skin. FIG. 2G depicts number of CD45+CD3+CD8+ T cells per gram of tissue in MC38 and B16 subcutaneous (SubQ) tumors, peritumoral tissues and healthy skin.

FIGS. 3A-3G. FIG. 3A depicts percentage of CD31+DARC+ blood endothelial cells (BEC) in pancreatic Panc02 tumors and pancreatic M8 organoid tumors in mice. FIG. 3B depicts number of CD31+DARC+ venular cells per gram of tissue in pancreatic Panc02 tumors and pancreatic M8 organoid tumors in mice. FIG. 3C depicts percentage of CD3+CD8+ T cells among all CD45+ cells in pancreatic Panc02 tumors and pancreatic M8 organoid tumors in mice. FIG. 3D depicts number of CD45+CD3+CD8+ T cells per gram of tissue in pancreatic Panc02 tumors and pancreatic M8 organoid tumors in mice. FIG. 3E depicts number of CD45+CD3+CD8+ T cells per gram of tissue in pancreatic Panc02 tumors in mice. FIG. 3F depicts number of CD45+CD3+CD8+ T cells per gram of tissue pancreatic M8 organoid tumors in mice. FIG. 3G depicts number of CD45+CD3+CD8+ T cells per gram of tissue in pancreatic Panc02 tumors and pancreatic M8 organoid tumors in mice.

FIGS. 4A-4F. FIG. 4A depicts percentage of CD31+DARC+ V-EC cells among BEC from human melanoma. FIG. 4B depicts number of DARC+ V-EC per gram of tissue from human melanoma. FIG. 4C depicts percent of CD45+CD3+CD8+ T cells among CD45+ cells from human melanoma. FIG. 4D depicts number of CD8+ T cells per gram of tissue from human melanoma. FIG. 4E depicts percent of CD45+CD3+CD8+ T cells among CD45+ cells from human melanoma. FIG. 4F depicts number of CD8+ T cells per gram of tissue from human melanoma.

FIGS. 5A-5B. FIG. 5A depicts a circle graph of the percentages of CD8+, CD4+, CD11c+CD103+, CD11c+CD103−, and CD11c− T cells in human skin sample and melanoma sample. FIG. 5B depicts the percentage of CD45-CD31+DARC+ V-EC among BEC in human skin tissue and melanoma tumor.

FIGS. 6A-6I. FIG. 6A depicts correlation between the number of CD8+ T cells and DARC+ VEC per gram of tissue in patient melanoma samples. R: patients who responded positively immunotherapy using anti PD1 or a combination of anti PD1 and anti CTL4 therapy. PD: patients with progressive disease, unresponsive to immunotherapy. FIG. 6B depicts the percentage of CD31+DARC+ V-EC of BEC in patient pancreatic tumor samples. FIG. 6C depicts number of CD31+CARD+ V-EC per gram in patient pancreatic tumor samples. FIG. 6D depicts percent of CD45+CD3+CD8+ T cells of CD45+ cells in patient pancreatic tumor samples. FIG. 6E depicts number of CD45+CD3+CD8+ T cells per gram in patient pancreatic tumor samples. FIG. 6F depicts correlation between the percentage of CD8+ T cells per amount of CD45+ cells and DARC+ VEC per amount of blood EC in patient pancreatic tumor samples. FIG. 6G depicts number of CD8+ T cells per gram in pancreatic tumor samples. FIG. 6H depicts comparison between pancreatic tumor, NM pancreas and duodenum. For each sample the percentage of CD8+ T cells per amount of CD45+ cells (open circle) and DARC+ VEC per amount of blood EC (full circle) were calculated and plotted using a line to connect them. FIG. 6I depicts homing experiment in RAG KO mouse. After MC38 and B16 tumors were allowed to grow in RAG KO mice, the mice were injected with fluorescent activated T cells. 24 hours after injection the number of CD8+ T cells per gram of tissue (open circle) and DARC+ VEC per gram of tissue (full circle) were calculated and plotted. A line to connect the data points form the same sample.

FIGS. 7A-7E. FIG. 7A depicts the correlation between the number of CD45+CD3+CD8+ T cells per gram of tissue and the number of CD31+DARC+ V-EC per gram of tissue in MC38 and B16F10 tumor models in mice. FIG. 7B depicts the correlation between the number of CD45+CD3+CD8+ T cells per gram of tissue and the number of CD31+DARC+ V-EC per gram of tissue in Panc02 and M8 tumors. FIG. 7C depicts the correlation of the number of CD8+ T cells per gram of tissue and the number of DARC+ V-EC per gram of tissue in human melanoma and pancreatic tumor tissues. FIG. 7D depicts the number of CD8+ T cells per gram of tissue in pancreatic and melanoma tissue samples. FIG. 7E depicts the number of DARC+ V-EC per gram of tissue in pancreatic and melanoma tissue samples.

FIGS. 8A and 8B. FIG. 8A depicts gating strategy for isolating CD8 T cells after differentiation of b-actin GFP splenocyte from a b-actin GFP mouse. Cells were cultured in anti-CD3 for 48 hours. Cells were washed in resuspended in media containing IL-2 (20 ng/mL) and used after 8-10 days. FIG. 8B depicts isolation of CD8+ cells from tumors 4 hours after transfer of differentiated b-actin GFP splenocytes.

FIGS. 9A-9F. FIG. 9A depicts processing pipeline. After sample collection (mouse or human), cells are dissociated and, for most samples, enriched for CD31+ cells using magnetic beads. Cells are then loaded on an array pre-loaded with sequencing beads. SeqWell protocol libraries were prepared and the data was analyzed. FIG. 9B depicts EC isolation: despite enriching for EC, many other cell types were identified. Therefore, using a combination of differential expression markers, EC scoring and cell type specific genes, EC were identified and isolated before proceeding with the next steps of the analysis. FIG. 9C depicts mouse healthy skin EC. Further, specialized EC subsets were identified based on previously validated gene markers for those subsets (FIG. 9D). FIG. 9E depicts human healthy skin EC and FIG. 9F depicts specialized EC subsets identified based on previously validated gene markers for those subsets.

FIGS. 10A-10C. FIG. 10A depicts a schematic of in silico gating of ECs. FIG. 10B depicts a sample of t-SNE of cells in healthy mouse skin. FIG. 10C depicts a sample of t-SNE and doublets overlay.

FIGS. 11A-11G. FIGS. 11A-1 to 11A-8 depict an iterative process to identify and select ECs in mouse healthy skin. In each iteration cell clusters containing ECs were identified based on cell markers, EC score and differentially expressed genes. First iteration: UMAP and EC score (FIG. 11A-1) and Darc, Pecam1, Cdh5, Lyve1 expression (FIG. 11A-2); Second iteration: UMAP and EC score (FIG. 11A-3) and Darc, Pecam1, Cdh5, Lyve1 expression (FIG. 11A-4); Third iteration: UMAP and EC score (FIG. 11A-5) and Darc, Pecam1, Cdh5, Lyve1 expression (FIG. 11A-6); and Fourth iteration: UMAP and EC score (FIG. 11A-7) and Darc, Pecam1, Cdh5, Lyve1 expression (FIG. 11A-8). FIG. 11B depicts healthy mouse skin ECs. Lymphatic ECs (LECs); non-venular ECs (NVECs); venular ECs (VECs). FIG. 11C depicts expression of EC genes, Darc, Pecam1, Cdh5, and Lyve1 overlaid on the UMAP (Uniform Manifold Approximation and Projection). FIG. 11D depicts expression of EC genes, Selp, Sele, Sdpr, and Vwf overlaid on the UMAP (Uniform Manifold Approximation and Projection). FIG. 11E depicts violin graph of VEC score. FIG. 11F depicts violin graph of Pecam1 expression. FIG. 11G depicts violin graph of Dare expression.

FIGS. 12A-12E. FIG. 12A depicts unbiased clustering (UMAP) and heatmap results in VEC and NVEC clusters from healthy skin. FIG. 12B depicts unbiased clustering (UMAP) and heatmap results in VEC and NVEC clusters from MC38 tumor samples. FIG. 12C depicts unbiased clustering (UMAP) and heatmap results in VEC and NVEC lusters from B16F10 tumor samples. VECs could not be identified by clustering alone. A VEC scoring with a cutoff of 0.2 to identify VECs. In the heatmap VECs were separated and put on the right to check their gene expression against the other clusters. FIG. 12D depicts in silico gating (unbiased clustering (UMAP)) of EC from B16F10 melanoma samples. FIG. 12E depicts a violin graph of each cluster based on VEC score from B16F10 samples in FIG. 12D. VEC scoring with a cutoff of 0.2 was used to identify VECs.

FIGS. 13A-131. FIG. 13A depicts unsupervised clustering of all mouse EC colored by cluster. FIG. 13B depicts unsupervised clustering of all mouse EC colored by cell and sample type. FIG. 13C depicts division of cell type by cluster. Heatmaps of top and bottom 50 differentially expressed genes in (FIG. 13D) Healthy skin VECs, (FIG. 13E) MC38 VECs, (FIG. 13F) B16 VECs. FIG. 13G depicts cell reassignment per cluster using the Silhouette algorithm. GSVA analysis of (FIG. 13H) MC38 VECs and (FIG. 13I) B16 VECs using a curated list of EC pathways. Bolded: pathways unique to MC38 or B16. Numbers in brackets: number of similar pathways.

FIGS. 14A-14F. depicts similarity scoring in mouse. FIG. 14A depicts healthy VEC, FIG. 14B depicts MC38 VEC, and FIG. 14C depicts B16 VEC using the VEC signature (top 50 upregulated genes in VECs) all cells were scored and compared. FIG. 14D depicts healthy VEC, FIG. 14E depicts MC38 VEC, and FIG. 14F depicts B16 VEC plotted on an axis. Here, for each sample type, a “VEC score” was calculated using the top 50 up-regulated genes and a “NVEC score” using the top 50 down-regulated genes. Those 2 scores were combined as follow: combined_score=(average_VEC_score_for_that_cluster)−(average_NVEC_score_for_that_cluster). Cells are then plotted on axis which ends up going from NVEC to VEC thus assessing the relative similarities of the subsets.

FIGS. 15A-15D. FIG. 15A depicts a UMAP of healthy human skin. FIG. 15B depicts in silico gating of EC from healthy human skin. FIG. 15C depicts expression of EC genes, Darc, CLDN5, PROX1, CDH5, and LYVE1 overlaid on the UMAP. FIG. 15D depicts a heatmap of healthy human skin.

FIGS. 16A-16E. FIG. 16A depicts heatmap of the gene signature in human healthy skin samples. FIG. 16B depicts heatmap of the gene signature in non-malignant pancreas VEC samples. FIG. 16C depicts heatmap of the gene signature in melanoma VEC samples. FIG. 16D depicts heatmap of the gene signature in pancreatic tumor VEC samples. FIG. 16E depicts cell reassignment per cluster using the Silhouette algorithm in human samples.

FIGS. 17A-17C. FIG. 17A depicts UMAPs and DotPlots of typical cell type markers for human Melanoma samples. FIG. 17B depicts UMAPs and DotPlots of typical cell type markers for human Non-malignant Pancreas samples. FIG. 17C depicts UMAPs and DotPlots of typical cell type markers for human Pancreatic tumor samples.

FIGS. 18A-18F. FIG. 18A depicts unsupervised clustering of human healthy skin and melanoma ECs colored by cluster and sample type. FIG. 18B depicts unsupervised clustering of human healthy skin and melanoma ECs colored by cell and sample type. FIG. 18C depicts division of cell type by cluster. FIG. 18D depicts unsupervised clustering of human (NM) pancreas and pancreatic tumor EC colored by cluster and sample type. FIG. 18E depicts unsupervised clustering of human (NM) pancreas and pancreatic tumor EC colored by cell and sample type. FIG. 18F depicts division of cell type by cluster.

FIGS. 19A-19D. FIG. 19A depicts the correlation between number of CD45+CD3+CD8+ T cells per gram of tissue and number CD31+DARC+ V-EC per gram of tissue in Panc02 and M8 tumors. FIG. 19B depicts the correlation between number of CD8+ T cells and number of DARC+ V-EC per gram of tissue in human melanoma and pancreatic tumor samples. FIG. 19C depicts the number of CD8+ T cells per gram of tissue from pancreatic and melanoma tumor samples. FIG. 19D depicts the number of DARC+ VEC per gram of tissue from pancreatic and melanoma tumor samples.

FIG. 20 depicts a heatmap of non-malignant NVEC, pancreatic tumor VEC, pancreatic tumor NVEC, human melanoma VEC, human melanoma NVEC, non-malignant pancreatic VEC, human skin VEC, and human skin NVEC tissue samples.

FIGS. 21A-21F. FIG. 21A depicts Venn diagrams of genes upregulated in immunogenic tumors. FIG. 21B depicts the top 10 enriched pathways in the shared gene list in immunogenic tumors using EnrichR. FIG. 21C depicts enriched transcription factors in immunogenic tumors. FIG. 21D depicts Venn diagrams of genes upregulated in non-immunogenic tumors. FIG. 21E depicts the top 10 enriched pathways in the shared gene list in non-immunogenic tumors using EnrichR. FIG. 21F depicts enriched transcription factors in non-immunogenic tumors. The combined score is a combination of the p-value and z-score calculated by multiplying the two scores as follows: c=ln(p)*z. Where c is the combined score, p is the p-value computed using Fisher's exact test, and z is the z-score computed to assess the deviation from the expected rank. The combined score provides a compromise between both methods and has been shown to reports the best rankings when compared with other scoring schemes.^{35,36}

FIGS. 22A-22D. FIG. 22A depicts DotPlots of transcription factors, TR1, TR2, and TR3, in various cells types, such as healthy skin (LEC, VEC, and NVEC), melanoma (NVEC, VEC, and LEC), healthy pancreas (LEC, NVEC, and VEC), and tumor pancreas (VEC and NVEC). FIG. 22B depicts DotPlots of transcription factors, TR1, TR2, and TR3, in various cells types, such as MC38 tumor (VEC and NVEC), B16 tumor (VEC and NVE), healthy skin (VEC, NVEC, and LEC). FIG. 22C depicts expression levels of mouse TR3 from all ECs in healthy skin, MC38 tumor, and B16 tumor cells. FIG. 22D depicts expression levels of human TR3 from all ECs in healthy skin, melanoma, non-malignant pancreas, and pancreatic tumor cells.

FIGS. 23A-23D. FIG. 23A depicts expression levels of mouse HIF1a in healthy skin cells (VEC, NVEC, and LEC), MC38 tumor cells (VEC, NVEC, and LEC), and B16F10 tumor cells (VEC, NVEC, and LEC). FIG. 23B depicts expression levels of mouse HIF1a from all ECs in healthy skin, MC38 tumor, and B16 tumor cells. FIG. 23C depicts expression levels of human HIF1a in healthy skin cells (VEC, NVEC, and LEC), melanoma tumor cells (VEC, NVEC, and LEC), non-malignant pancreas cells (VEC, NVEC, and LEC), and pancreatic tumor cells (VEC, NVEC, and LEC). FIG. 23D depicts expression levels of human HIF1a from all ECs in healthy skin, melanoma tumor, non-malignant pancreas, and pancreatic tumor cells.

FIGS. 24A-24B. FIG. 24A depicts tumor size (volume mm³) from MC38 tumors implanted in wild type and TR3 knockout mice. FIG. 24B isolation of CD31+DARC+ cells from MC38 tumors implanted in wild type and TR3 knockout mice.

FIGS. 25A-25D. FIG. 25A depicts percentage of CD31+DARC+ BACs per gram of tissue in MC38 tumors and peritumoral tissues from wild type and TR3 knockout mice. FIG. 25B depicts number of CD31+DARC+ BACs per gram of tissue in MC38 tumors and peritumoral tissues from wild type and TR3 knockout mice. FIG. 25C depicts percentage of CD31+DARC-NVEC BACs per gram of tissue in MC38 tumors and peritumoral tissues from wild type and TR3 knockout mice. FIG. 25D depicts number of CD31+DARC-BACs per gram of tissue in MC38 tumors and peritumoral tissues from wild type and TR3 knockout mice.

FIGS. 26A-26C depicts Venn diagrams of up-regulated genes in murine and human tumor microvasculature compared to healthy tissues. Single cell suspensions of murine MC38 colorectal adenocarcinoma (MC38, blue), murine B16F10 melanoma (B16, red), human pancreatic cancer (hPanT, yellow), human melanoma (hMel, green) and peri-tumoral tissue were isolated by Seq Well and processed for scRNA-seq. V-ECs and NV-ECs were identified based on characteristic gene expression patterns and each EC subset in tumors and matched peri-tumoral tissue was compared to identify tumor-specific over-expressed genes. Venn diagrams of the number of upregulated genes as compared to healthy in FIG. 26A) all blood ECs, FIG. 26B) NV-ECs and FIG. 26C) V-ECs.

FIGS. 27A-27E. FIGS. 27A and 27B depicts validation assays of a candidate tumor EC target, PMEPA1. EC mRNA levels of PMEPA1 were compared in VEC, NVEC and lymphatic EC (LEC) in (FIG. 27A) healthy mouse skin and subcutaneous MC38 and B16F10 tumors and (FIG. 27B) human non-malignant pancreas and pancreatic cancer. FIG. 27C FACS analysis of PMEPA1 on ECs in MC38 tumors and healthy skin. FIG. 27D FACS analysis of PMEPA1 on ECs in MC38 tumors and healthy skin using Iso control antibody. FIG. 27E Percentage of PMEPA1+ blood EC (BEC).

FIGS. 28A-28D depicts the generation of nanobody against PMEPA1. FIG. 28A L1.2 cells were transfected with either a linearized or a circular plasmid. FIG. 28B The cells were expanded in the presence of G418 and GFP expression was assessed by FACS. FIG. 28C Cells with the highest MFI were single sorted and expanded. FIG. 28D Clones demonstrating the highest level of PMEPA1-GFP expression (which is enhanced by treatment with sodium butyrate) is ready for use in selection of sdAb from the yeast display library.

FIG. 29 shows enrichment of receptor positive cells. Receptor-negative and receptor-positive cells were labeled with different fluorescent dyes and mixed in 1:1 ratio. Yeast expressing sdAb library was added to the cultures in proportion Yeast:Targets=25:1. The cultures were incubated on a gentle shaker at 4° C. for 1 h, the cells were then collected and stained for FACS to determine the percentage of HA+ cells in each population.

FIG. 30 shows a schematic strategy to generate sdAb against PMEPA1 to target CAR-T cells to solid tumors.

### DETAILED DESCRIPTION

The present disclosure provides compositions and methods for treating solid tumors by targeting clinically relevant molecules that are upregulated in tumor cells. For example, the present disclosure provides compositions and methods for treating solid tumors by targeting clinically relevant molecules that are upregulated in tumor vascular endothelial cells, such as non-venular and venular endothelial cells in and surrounding a tumor, forming the tumor microvasculature, but that are not upregulated in vasculature from healthy tissues, e.g., non-tumor vascular endothelial cells. Characterization of tumor microvasculature, human and murine immune cell infiltrates of immunogenic tumors (T-cell rich and onco-immunotherapy responders), and nonimmunogenic tumors (T-cell poor and onco-immunotherapy non-responders) demonstrated the importance of the vasculature in recruiting intra-tumoral T cells and allowed for the identification of genes that are over-represented in tumor microvasculature of solid tumors, globally in all tumor vascular endothelial cells within the tumors or selectively in venules or non-venules (capillaries and arterioles).

Thus, the present disclosure provides for methods and compositions i) for targeting overexpressed venular surface molecules identified from both immunogenic tumors and non-immunogenic tumors to target the venules with gene and/or drug delivery for tumor vascular endothelial cell re-programing to increase intra-tumoral T cells, and/or ii) for targeting overexpressed venular surface molecules identified from solid tumors to specifically target oncotherapeutic agents (e.g., chemotherapeutic agent or CAR T cells) to venules or non-venules selectively in human tumors with minimum off target effects. The present disclosure also provides targeting molecules, such as nanobodies, that target the overexpressed surface molecules found on tumor vascular endothelial cells.

### Definitions

In order that the present invention may be more readily understood, certain terms are first defined.

Unless otherwise defined herein, scientific and technical terms used in connection with the present invention shall have the meanings that are commonly understood by those of ordinary skill in the art. The meaning and scope of the terms should be clear, however, in the event of any latent ambiguity, definitions provided herein take precedent over any dictionary or extrinsic definition.

The use of the terms “a” and “an” and “the” and similar referents in the context of describing the invention (especially in the context of the following claims) are to be construed to cover both the singular and the plural (i.e., one or more), unless otherwise indicated herein or clearly contradicted by context. The terms “comprising, “having,” “including,” and “containing” are to be construed as open-ended terms (i.e., meaning “including, but not limited to”) unless otherwise noted. Recitation of ranges of values herein are merely intended to serve as a shorthand method of referring individually to each separate value recited or falling within the range, unless otherwise indicated herein, and each separate value is incorporated into the specification as if it were individually recited.

The term “about” or “approximately” means within 5%, or more preferably within 1%, of a given value or range.

As used herein, the term “encode” or “encoding” refers to a property of sequences of nucleic acids, such as a vector, a plasmid, a gene, cDNA, mRNA, to serve as templates for synthesis of other molecules such as proteins.

The terms “increased,” “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein, the term “substantially” refers to the qualitative condition of exhibiting total or near-total extent or degree of a characteristic or property of interest. One of ordinary skill in the art will understand that biological and chemical phenomena rarely, if ever, go to completion and/or proceed to completeness or achieve or avoid an absolute result. The term “substantially” may therefore be used in some embodiments herein to capture potential lack of completeness inherent in many biological and chemical phenomena.

It should be noted that whenever a value or range of values of a parameter are recited, it is intended that values and ranges intermediate to the recited values are also intended to be part of this invention.

As used herein, the term “tumor vascular endothelial cell” refers to the endothelial cells that are associated with a tumor. Vascular endothelial cells form the lining of the inner surface of all blood vessels, and constitute a non-thrombogenic interface between blood and tissue. In addition, vascular endothelial cells are an important component for the development of new capillaries and blood vessels. Tumor vascular endothelial cells proliferate during the angiogenesis, or neovascularization, associated with tumor growth and metastasis. Tumor vascular endothelial cells are associated with new capillaries and blood vessels associated with tumors. (See Dudley A C. Tumor endothelial cells. Cold Spring Harb Perspect Med. 2012; 2 (3): a006536. doi: 10.1101/cshperspect.a006536). The tumor vascular endothelial cells include both venular and non-venular endothelial cells found in or surrounding tumor.

As used herein, the term “non-tumor vascular endothelial cell” refer to endothelial cells that are not associated with a tumor and, for example, are found in normal “healthy” tissues. The vascular endothelium is a dynamic cellular “organ” that controls passage of nutrients into tissues, maintains the flow of blood, and regulates the trafficking of leukocytes (e.g., T cell). In normal tissues, the endothelial cells form a continuous and uniform monolayer, while tumor endothelial cells are irregular in shape and size and have cytoplasmic projection extending into the vessel lumen. Tumor vascular endothelial cells can block T cells from entry into the tumor through the deregulation of adhesion molecules in the vessels. (See Lanitis E, Irving M, Coukos G. Targeting the tumor vasculature to enhance T cell activity. Curr Opin Immunol. 2015; 33:55-63. doi:10.1016/j.coi.2015.01.011).

As used herein, the term “tumor stroma” refers to a heterogeneous component of a tumor microenvironment. The “tumor stroma” is made up of noncellular and cellular components such as the extracellular matrix, the tumor-vasculature and tumor stromal cells.

As used herein, the term “tumor stromal cell” refers to a non-cancerous cell and non-immune cell within a tumor, and the tumor stromal cell is within the “tumor stroma.” Tumor stromal cells include connective tissue cells such as fibroblasts, e.g., cancer-associated fibroblasts, mesenchymal stromal cells, and pericytes. In solid tumors, the stromal cells interact with neoplastic cells to influence the behavior of a tumor.

As used herein, the term “non-tumor stromal cells” refers to stromal cells that are not associated with a tumor and, for example, are found in normal “healthy” tissues.

### Targeting Molecules

As used herein, a “targeting molecule” refers to any molecule that binds to a component associated with an organ, tissue, cell, extracellular matrix, and/or subcellular locale. In some embodiments, such a component is referred to as a “target” or a “marker”.

A targeting molecule may be a nucleic acid, polypeptide, glycoprotein, carbohydrate, lipid, small molecule, etc. For example, a targeting molecule can be a nucleic-acid targeting molecule (e.g., an aptamer, Spiegelmer®, etc.) that binds to a cell type specific marker. In general, an aptamer is an oligonucleotide (e.g., DNA, RNA, or an analog or derivative thereof) that binds to a particular target, such as a polypeptide. In some embodiments, a targeting molecule may be a naturally occurring or synthetic ligand for a cell surface receptor, e.g., a growth factor, hormone, LDL, transferrin, etc. A targeting molecule can be an antibody, which term is intended to include antibody fragments, characteristic portions of antibodies, single chain antibodies, etc. Synthetic binding proteins such as Affibodies®, Nanobodies™, AdNectins™, Avimers™, etc., can be used. In a particular embodiment, the targeting molecule is a nanobody. Peptide targeting molecule can be identified, e.g., using procedures such as phage display. This widely used technique has been used to identify cell specific ligands for a variety of different cell types.

In accordance with the present invention, a targeting molecule recognizes one or more “targets” or “markers” associated with a particular organ, tissue, cell, and/or subcellular locale. In some embodiments, a target may be a marker that is exclusively or primarily associated with one or a few cell types, with one or a few diseases, and/or with one or a few developmental stages.

In a particular embodiment, the target is a transmembrane molecule on tumor vascular endothelial cells. In some embodiments, the transmembrane molecule is upregulated on the tumor vascular endothelial cells as compared expression on a non-tumor vascular endothelial cell. In some embodiments, the transmembrane molecule is selected from the molecules listed in Tables 8-10. In some embodiments, the tumor vascular endothelial cell is a venular cell.

The transmembrane molecule (e.g., selected from the molecules listed in Table 8-10) is typically expressed at levels at least 1.5-fold, at least 2-fold, at least 2.5-fold, at least 3-fold, at least 3.5-fold, at least 4-fold, at least 4.5-fold, or at least 5-fold greater in tumor vascular endothelial cells than in a reference population of cells (e.g., non-tumor vascular endothelial cell) which may consist, for example, of a mixture containing an approximately equal amount of cells (e.g., approximately equal numbers of cells, approximately equal volume of cells, approximately equal mass of cells, etc.). In some embodiments, the transmembrane molecule is present at levels at least 1.5 fold, at least 2 fold, at least 3 fold, at least 4 fold, at least 5 fold, at least 6 fold, at least 7 fold, at least 8 fold, at least 9 fold, at least 10 fold, at least 50 fold, at least 100 fold, at least 500 fold, at least 1000 fold, at least 5000 fold, or at least 10,000 fold greater than its average expression in a reference population. Detection or measurement of the transmembrane molecule may make it possible to distinguish the cell type or types of interest from cells of many, most, or all other types.

In some embodiments, targeting molecules are coupled (e.g., covalently associated) with an agent that is capable of inducing cell death to a tumor vascular endothelial cell in which the expression of at least one transmembrane molecule (e.g., a transmembrane molecule from Tables 8-10) is upregulated as compared to a non-tumor vascular endothelial cell control cell. For example, the agent may be capable of inducing immunogenic cell (e.g., cancer cell) death whereby a subsequent immune response is elicited upon the cell death. Alternatively, the agent may be capable of inducing a non-immunogenic cell (e.g., cancer cell) death, whereby a subsequent immune response is not elicited upon cell death.

In some embodiments, covalent association is mediated by a linker. In some embodiments, targeting molecules are not covalently associated with the agent that is capable of inducing cell death to an tumor vascular endothelial cell in which the expression of at least one transmembrane molecule (e.g., a transmembrane molecule from Tables 8-10) is upregulated as compared to a non-tumor vascular endothelial cell control cell. For example, targeting molecules may be associated with the surface of, encapsulated within, surrounded by, and/or distributed throughout the lipid formulation or polymeric matrix of an lipid nanoparticle, nanosphere, nanocarrier, microsphere, or microparticle. For example, in some embodiments, a targeting molecule can be encapsulated within, surrounded by, and/or dispersed throughout the liposomal membrane and/or polymeric matrix of a lipid nanoparticle, a nanosphere, a nanocarrier, a microsphere or a microparticle. Alternatively or additionally, a targeting molecule can be associated with a lipid nanoparticle or a nanocarrier by charge interactions, affinity interactions, metal coordination, physical adsorption, host-guest interactions, hydrophobic interactions, TT stacking interactions, hydrogen bonding interactions, van der Waals interactions, magnetic interactions, electrostatic interactions, dipole-dipole interactions, and/or combinations thereof.

The nanoparticles, nanospheres, nanocarrier, microparticles or microspheres may comprise one or more of polysaccharides, proteins, lipids, chitosan, alginate, pectin, xanthan gum, and cellulose. The nanoparticles, nanospheres or nanocarriers may be liposomes, polymerie micelles, dendrimers. Exemplary dendrimers include those comprising poly-L-lysine, polyamidoamine (PAMAM), polypropylene imine (PPI), liquid crystalline, core-shell, chiral, peptide, glycodendrimers and PAMAMOS dendrimers. Alternatively, the nanoparticles, nanospheres, nanocarrier, microparticles or microspheres may comprise an inorganic compound such as silver, gold, iron oxide, silica, zinc oxide, titanium oxide, platinum, selenium, gadolinium, palladium, or cerium dioxide. In some embodiments, the targeting molecule is covalently linked to a lipid nanoparticle, nanosphere, nanocarrier, microsphere or microparticle. For example, the targeting molecule is linked to a nanoparticle, nanosphere, nanocarrier, microsphere or microparticle by a peptide linker. Exemplary peptide linkers include the dipeptide Val Cit (VC), the tripeptide AAN, or longer peptide such as (GGGGS) (n=1, 2, 3, or 4) (SEQ ID NO: 51), (Gly)8 (SEQ ID NO: 52), (Gly)6 (SEQ ID NO: 53), (EAAAK)3 (SEQ ID NO: 54), (EAAAK) (n=1-3) (SEQ ID NO: 55), A(EAAAK)4ALEA(EAAAK)4A (SEQ ID NO: 56), PAPAP (SEQ ID NO: 57), AEAAAKEAAAKA (SEQ ID NO: 58), (Ala-Pro)n (10-34 aa) (SEQ ID NO: 59). Other types of linkers include GPI-anchors and cross-linked polymers.

Alternatively, the targeting molecule is linked to a nanoparticle, nanosphere, nanocarrier, microsphere or microparticle by a cleavable linker such as an acid-labile linker, a protease cleavable linker, an enzyme cleavable linker, or a reducible disulfide linkage. Exemplary cleavable linkers include those comprising an ester bond such as a glutaryl linker, those comprising an amide bond and those comprising a carbamate bond. An exemplary acid-labile linker are hydrozone linkers.

In addition, the targeting molecule is linked to a nanoparticle, nanosphere, nanocarrier, microsphere or microparticle by an uncleavable such as an amide bond and a succinimidyl thioester linker or an amide bond and triazole linker or an oxime linker or a triazole linker.

In some embodiments, a targeting molecule in accordance with the present invention may be a protein or peptide. In certain embodiments, peptides range from about 5 to about 100, from about 5 to about 50, from about 10 to about 75, from about 15 to about 50, or from about 20 to about 25 amino acids in size. In some embodiments, a peptide sequence can be based on the sequence of a protein. In some embodiments, a peptide sequence can be a random arrangement of amino acids.

The terms “polypeptide” and “peptide” are used interchangeably herein, with “peptide” typically referring to a polypeptide having a length of less than about 100 amino acids. Polypeptides may contain L-amino acids, D-amino acids; or both and may contain any of a variety of amino acid modifications or analogs known in the art. Useful modifications include, e.g., terminal acetylation, amidation, lipidation, phosphorylation, glycosylation, acylation, farnesylation, sulfation, etc.

Exemplary proteins that may be used as targeting molecules in accordance with the present invention include, but are not limited to, antibodies, receptors, cytokines, peptide hormones, glycoproteins, glycopeptides, proteoglycans, proteins derived from combinatorial libraries (e.g., Avimers™, Affibodies®, etc.), and characteristic portions thereof. Synthetic binding proteins such as Nanobodies™, AdNectins™, etc., can be used. In some embodiments, protein targeting molecules can be a nanobody.

One of ordinary skill in the art will appreciate that any protein and/or peptide that specifically binds to a desired target as described herein, can be used in accordance with the present invention.

In some embodiments, a targeting molecule may be an antibody and/or characteristic portion thereof. The term “antibody” refers to any immunoglobulin, whether natural or wholly or partially synthetically produced and to derivatives thereof and characteristic portions thereof. An antibody may be monoclonal or polyclonal. An antibody may be a member of any immunoglobulin class, including any of the human classes: IgG, IgM, IgA, IgD, and IgE.

As used herein, an antibody fragment (i.e. characteristic portion of an antibody) refers to any derivative of an antibody which is less than full-length. In some embodiments, an antibody fragment retains at least a significant portion of the full-length antibody's specific binding ability. Examples of such antibody fragments include, but are not limited to, Fab, Fab′, F(ab′)2, scFv, Fv, dsFv diabody, and Fd fragments. Antibody fragments also include, but are not limited, to Fc fragments.

An antibody fragment may be produced by any means. For example, an antibody fragment may be enzymatically or chemically produced by fragmentation of an intact antibody and/or it may be recombinantly produced from a gene encoding the partial antibody sequence. Alternatively or additionally, an antibody fragment may comprise multiple chains which are linked together, for example, by disulfide linkages. An antibody fragment may optionally comprise a multimolecular complex. A functional antibody fragment will typically comprise at least about 50 amino acids and more typically will comprise at least about 200 amino acids.

In some embodiments, antibodies may include chimeric (e.g. “humanized”) and single chain (recombinant) antibodies. In some embodiments, antibodies may have reduced effector functions and/or bispecific molecules. In some embodiments, antibodies may include fragments produced by a Fab expression library.

Nanobodies are recombinant antibody fragments consisting of one variable heavy chain. In some embodiments, the variable heavy chain of a nanobody comprises a CDR1, CDR2, and CDR3. The CDR1 and CDR2 segments can be short in comparison to the CDR3 segment, which is longer than the typical CDR3 in a conventional antibody or scFv molecule.

In some embodiments, the nanobodies can comprise multiple (two or more) VH segments, such as a dimer. Peptide linker can be between VH segments. Each VH segment in a multimer nanobody can be the same VH sequence binding to the same antigen, or different VH sequence binding to different antigens, or different VH sequences binding the same antigen at non-overlapping epitopes. In some embodiments, the nanobodies can comprise multiple segments of VH segments as described above and scFv molecules.

In some embodiments, the nanobodies can be covalently linked to a drug (e.g., chemotherapeutic drug), imaging probe, or displayed on the surface of nanoparticles, viruses, or CAR T cells.

In some embodiment, the antibody or antigen-binding fragment thereof is covalently linked to one or more detectable markers (e.g., imaging probe or detectable labels) or other signal-generating groups or moieties, depending on the intended use of the labeled nanobody. Suitable markers and techniques for attaching, using and detecting them will be clear to the skilled person and, for example, include, but are not limited to, fluorescent labels (such as fluorescein, isothiocyanate, rhodamine, phycoerythrin, phycocyanin, allophycocyanin, o-phthaldehyde, and fluorescamine and fluorescent metals such as Eu or others metals from the lanthanide series), phosphorescent labels, chemiluminescent labels or bioluminescent labels (such as luminal, isoluminol, theromatic acridinium ester, imidazole, acridinium salts, oxalate ester, dioxetane or GFP and its analogs), radio-isotopes, metals, metals chelates or metallic cations or other metals or metallic cations that are particularly suited for use in in vivo, in vitro or in situ diagnosis and imaging, as well as chromophores and enzymes (such as malate dehydrogenase, staphylococcal nuclease, delta-V-steroid isomerase, yeast alcohol dehydrogenase, alpha-glycerophosphate dehydrogenase, triose phosphate isomerase, biotinavidin peroxidase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholine esterase). Other suitable labels will be clear to the skilled person and, for example, include moieties that can be detected using NMR or ESR spectroscopy.

Single-chain Fvs (scFvs) are recombinant antibody fragments consisting of only the variable light chain (VL) and variable heavy chain (VH) covalently connected to one another by a polypeptide linker. Either VL or VH may comprise the NH2-terminal domain. The polypeptide linker may be of variable length and composition so long as the two variable domains are bridged without significant steric interference. Typically, linkers primarily comprise stretches of glycine and serine residues with some glutamic acid or lysine residues interspersed for solubility.

Diabodies are dimeric scFvs. Diabodies typically have shorter peptide linkers than most scFvs, and they often show a preference for associating as dimers.

An Fv fragment is an antibody fragment which consists of one VH and one VL domain held together by noncovalent interactions. The term “dsFv” as used herein refers to an Fv with an engineered intermolecular disulfide bond to stabilize the VH-VL pair.

An F(ab′)2 fragment is an antibody fragment essentially equivalent to that obtained from immunoglobulins by digestion with an enzyme pepsin at pH 4.0-4.5. The fragment may be recombinantly produced.

A Fab′ fragment is an antibody fragment essentially equivalent to that obtained by reduction of the disulfide bridge or bridges joining the two heavy chain pieces in the F(ab′)2 fragment. The Fab′ fragment may be recombinantly produced.

A Fab fragment is an antibody fragment essentially equivalent to that obtained by digestion of immunoglobulins with an enzyme (e.g., papain). The Fab fragment may be recombinantly produced. The heavy chain segment of the Fab fragment is the Fd piece.

Agents that Induce Cell Death or Inflammatory Response

Cell death can be classified according to the morphological appearance of the lethal process (that may be apoptotic, necrotic, autophagic or associated with mitosis), enzymological criteria (with and without the involvement of nucleases or distinct classes of proteases, like caspases), functional aspects (programmed or accidental, physiological or pathological) or immunological characteristics (immunogenic or non-immunogenic) (Kroemer et al., 2009).

Agents that induce cell (e.g., cancer cell) death, also referred to herein as “cell death stimulating agents” may be immunogenic or non-immunogenic in nature.

As used herein, the term “immunogenic cell death” or “immunogenic apoptosis” refers to dying cells that alert the immune system, which then mounts a therapeutic anti-cancer immune response and contributes to the eradication of residual tumor cells. Conversely, when cancer cells succumb to a non-immunogenic death modality, i.e., non-immunogenic cell death, they fail to elicit such a protective immune response.

As used herein, the term “anti-cancer immune response” refers to when an immune response is directed against tumor cells, in particular cancerous cells. The anti-cancer immune response is allowed by a reaction from the immune system of the subject to the presence of cells, preferably of tumor cells, dying from an immunogenic cell death (as defined previously).

As used herein, the terms “agent that induces an immunogenic cell death” or “immunogenic cell death stimulating agents” refer to an agent that induces an immunogenic cell death which then in turn induces an anti-cancer immune response. In some embodiments, targeting molecules may target and/or transport one or more immunogenic cell death stimulating agents (e.g., an agent that induces an immunogenic cell death, e.g., chemotherapeutic agent or CAR T cells) which can help stimulate immune responses. In some embodiments, immunogenic cell death stimulating agents boost immune responses by activating APCs to enhance their immunostimulatory capacity. In some embodiments, immunogenic cell death stimulating agents boost immune responses by amplifying lymphocyte responses to specific antigens. In some embodiments, immunogenic cell death stimulating agents boost immune responses by inducing the local release of mediators, such as cytokines from a variety of cell types. In some embodiments, the immunogenic cell death stimulating agents suppress or redirect an immune response. In some embodiments, the immunogenic cell death stimulating agents induce regulatory T cells. In some embodiments, the immunogenic cell death stimulating agents increase the levels or activity of intra-tumoral T cells.

As used herein, the term “agent that induces a non-immunogenic cell death” refers to an agent that induces cell death, but fails to elicit a corresponding protective immune response in doing so.

In some embodiments, the term “agent that induces an inflammatory response” refers to an agent that induces an inflammatory response which in turn induces a pro-inflammatory cytokine cascade. Cytokines activate immune cells such as T cells and macrophages, stimulating them to produce more cytokines resulting in so-called cytokine storms or cascades. In some embodiments, the agent that induces an inflammatory response is a TLR4 agonist or a GP-130 agonist.

In some embodiments, the agent is selected from the group consisting of a small molecule, saccharide, oligosaccharide, polysaccharide, peptide, protein, peptide analog and derivatives, peptidomimetic, siRNAs, shRNAs, antisense RNAs, ribozymes, dendrimers, aptamers, and any combination thereof.

In some embodiments, the targeting molecules and cell death stimulating agents, e.g., immunogenic or non-immunogenic cancer cell death stimulating agent, or inflammatory response stimulating agents are coupled (e.g., covalently associated or within the same structure, e.g., within a nanoparticle or the targeting molecule is decorating the cell membrane of a CAR T cell). In some embodiments, a nanoparticle comprises a lipid membrane (e.g., lipid bilayer, lipid monolayer, etc.), wherein at least one type of cell death stimulating agent is associated with the lipid membrane of the nanoparticle and at least one targeting molecule is associated with the lipid membrane of the nanoparticle. In some embodiments, at least one type of cell death stimulating agent is embedded within the lipid membrane of the nanoparticle and at least one targeting molecule is embedded within the lipid membrane of the nanoparticle. In some embodiments, the at least type of cell death stimulating agent is encapsulated by the lipid membrane of the nanoparticle and the at least targeting molecule is associated and/or embedded in the lipid membrane or the nanoparticle.

In some embodiments, the at least one type of cell death stimulating agent, e.g., immunogenic or non-immunogenic cancer cell death stimulating agent, is associated with the interior surface of the lipid membrane of the nanoparticle and the targeting molecule is associated with the exterior surface of the lipid membrane of the nanoparticle. In some embodiments, the at least one type of cell death stimulating agent may be located at multiple locations of a nanoparticle. For example, a first type of cell death stimulating agent may be embedded within a lipid membrane, and a second type of cell death stimulating agent may be encapsulated within the lipid membrane of a nanoparticle. To give another example, a first type of cell death stimulating agent may be associated with the exterior surface of a lipid membrane, and a second type of cell death stimulating agent may be associated with the interior surface of the lipid membrane of a nanoparticle. In some embodiments, a first type of cell death stimulating agent may be embedded within the lipid bilayer of a nanoparticle, and the lipid bilayer may encapsulate a polymeric matrix throughout which a second type of cell death stimulating agent is distributed. In some embodiments, a first type of cell death stimulating agent and a second type of cell death stimulating agent may be in the same locale of a nanoparticle (e.g., they may both be associated with the exterior surface of a nanoparticle; they may both be encapsulated within the nanoparticle; etc.). One of ordinary skill in the art will recognize that the preceding examples are only representative of the many different ways in which multiple cell death stimulating agents or inflammatory response stimulating agents may be associated with different locales of nanoparticles. Multiple cell death stimulating agents or inflammatory response stimulating agents may be located at any combination of locales of nanoparticles.

In certain embodiments, cell death stimulating agents, e.g., immunogenic or non-immunogenic cancer cell death stimulating agent, or inflammatory response stimulating agents may be interleukins, interferon, cytokines, etc. In specific embodiments, an cell death stimulating agent may be a natural or synthetic agonist for a Toll-like receptor (TLR). In specific embodiments, nanoparticles incorporate a ligand for toll-like receptor (TLR)-7, such, as CpGs, which induce type I interferon production. In specific embodiments, an cell death stimulating agent may be an agonist for the DC surface molecule CD40. In certain embodiments, to stimulate immunity rather than tolerance, a nanoparticle incorporates an cell death stimulating agent that promotes DC maturation (needed for priming of naive T cells) and the production of cytokines, such as type I interferons, which promote antibody responses and anti-viral immunity. In some embodiments, an cell death stimulating agent may be a TLR-4 agonist, such as bacterial lipopolysaccharide (LPS), VSV-G, and/or HMGB-1. In some embodiments, cell death stimulating agent are cytokines, which are small proteins or biological factors (in the range of 5 kD-20 kD) that are released by cells and have specific effects on cell-cell interaction, communication and behavior of other cells. In some embodiments, cell death stimulating agent may be proinflammatory stimuli released from necrotic cells (e.g., urate crystals). In some embodiments, cell death stimulating agents or inflammatory response stimulating agents may be activated components of the complement cascade (e.g., CD21, CD35, etc.). In some embodiments, cell death stimulating agents or inflammatory response stimulating agents may be activated components of immune complexes. The cell death stimulating agents include TLR-1, TLR-2, TLR-3, TLR-4, TLR-5, TLR-6, TLR-7, TLR-8, TLR-9, and TLR-10 agonists. The inflammatory response stimulating agents include, but are not limited to, TLR-4 agonist and GP-130 agonist. The cell death stimulating agents also include complement receptor agonists, such as a molecule that binds to CD21 or CD35. In some embodiments, the complement receptor agonist induces endogenous complement opsonization of the nanocarrier, cell death stimulating agents also include cytokine receptor agonists, such as a cytokine. In some embodiments, the cytokine receptor agonist is a small molecule, antibody, fusion protein, or aptamer.

In some embodiments, there are more than one type of cell death stimulating agent, e.g., immunogenic and/or non-immunogenic cancer cell death stimulating agent. In some embodiments, the different cell death stimulating agents or different inflammatory response stimulating agents each act on a different pathway. The cell death stimulating agents, therefore, can be different Toll-like receptors, a Toll-like receptor and CD40, a Toll-like receptor and a component of the inflammasome, etc.

In some embodiments, the cell death stimulating agents or inflammatory response stimulating agents may be an adjuvant. Thus, in some embodiments, the present invention provides pharmaceutical compositions comprising nanoparticles formulated with one or more adjuvants. The term “adjuvant”, as used herein, refers to an agent that does not constitute a specific antigen, but boosts the immune response to the administered antigen.

In some embodiments, the present invention is directed to delivery of adjuvant using nanoparticles capable of carrying the adjuvant (encapsulated and/or on a surface) to targeted locations such as a tumor vascular endothelial cell, wherein the nanoparticle comprises: (i) one or more molecules on a surface to target a specific cell; (iii) one or more molecules that are capable of eliciting an cell death when covalently attached to a polymer or encapsulated inside the nanoparticles. The embodiment is directed to enhancing the potentiating of an immune response in a mammal, comprising administering art effective amount of a nanoparticle delivery of adjuvant of the present invention to enhance the immune response of a mammal to one or more antigens.

For example, in some embodiments, the adjuvant is encapsulated within the nanoparticles of the invention. Typically, in such cases, the adjuvant is present in free form, i.e., the adjuvant is not conjugated to the polymers that form the nanoparticles. Adjuvant is encapsulated during the preparation of the nanoparticles in the usual manner, as exemplified herein. The release profile of the adjuvant from the nanoparticles when administered to a patient will depend upon a variety of factors, including the size of the nanoparticles, rate of dissolution of the polymer forming the nanoparticles (if dissolution occurs), the molecular weight of the polymer forming the nanoparticles, and the chemical characteristics of the adjuvant (which, in turn, will influence the location of the adjuvant within the nanoparticles, diffusion rates, etc.). The amount of adjuvant encapsulated in the polymer nanoparticles will be determined during the process of formation of the nanoparticles.

For example, in some embodiments, the adjuvant is conjugated to the polymers that form the nanoparticles. Typically, in such cases, the adjuvant is expressed on or near the surface of the nanoparticles. In some embodiments, an amphilic polymer capable of self-assembling into nanoparticles is used, and the adjuvant is covalently attached to one terminus of the polymer. For example, the adjuvant may be used as an initiating species in the polymerization reaction used to form the polymers. When the adjuvant is conjugated (i.e., covalently bonded) to a terminus of the polymer, upon self-assembly of the polymer, the adjuvant is concentrated at the periphery or at the core of the nanoparticles. For example, in a polymer comprising a hydrophobic block and a hydrophilic block, wherein the polymer is allowed to self-assemble into nanoparticles having a hydrophobic core and a hydrophilic periphery, adjuvant that is conjugated to the terminus of the hydrophilic block will be concentrated at the periphery of the nanoparticles. In some preferred embodiments, the adjuvant is concentrated at the surface of the nanoparticles and remains in a position to act as an immunostimulant. For nanoparticle formulations comprising conjugated adjuvant, the density of adjuvant on the surface of the nanoparticles will be a function of a variety of factors, including the molecular weight of the polymers forming the nanoparticles, the density of the nanoparticles, and the chemical characteristics of the adjuvant.

In some embodiments, a combination of encapsulated and conjugated adjuvant is used.

For example, in some embodiments, nanoparticles are formulated with one or more adjuvants such as gel-type adjuvants (e.g., aluminum hydroxide, aluminum phosphate, calcium phosphate, etc.), microbial adjuvants (e.g., immunomodulatory DNA sequences that include CpG motifs; endotoxins such as monophosphoryl lipid A; exotoxins such as cholera toxin, E. coli heat labile toxin, and pertussis toxin; muramyl dipeptide, etc.); oil-emulsion and emulsifier-based adjuvants (e.g., Freund's Adjuvant, MF59 [Novartis], SAF, etc.); particulate adjuvants (e.g., liposomes, biodegradable microspheres, saponins, etc.); synthetic adjuvants (e.g., nonionic block copolymers, muramyl peptide analogues, polyphosphazene, synthetic polynucleotides, etc.), surfactant based adjuvants, and/or combinations thereof. Other exemplary adjuvants include some polymers (e.g., polyphosphazenes, described in U.S. Pat. No. 5,500,161, which is incorporated herein by reference), QS21, squalene, tetrachlorodecaoxide, etc.

The term “adjuvant” is intended to include any substance which is incorporated into or administered simultaneously with the conjugates of the invention and which nonspecifically potentiates the immune response in the subject. Adjuvants include aluminum compounds, e.g., gels, aluminum hydroxide and aluminum phosphate; and Freund's complete or incomplete adjuvant (in which the conjugate is incorporated in the aqueous phase of a stabilized water in paraffin oil emulsion). The paraffin oil may be replaced with different types of oils, e.g., squalene or peanut oil. other materials with adjuvant properties include BCG (attenuated Mycobacterium tuberculosis), calcium phosphate, levamisole, isoprinosine, polyanions (e.g., poly A:U) leutinan, pertussis toxin, choler toxin, lipid A, saponins and peptides, e.g., muramyl dipeptide. Rare earth salts, e.g., lanthanum and cerium, may also be used as adjuvants. The number and/or amount of adjuvants depends on the subject and the particular conjugate used and can be readily determined by one skilled in the art without undue experimentation. The adjuvant to be incorporated in the nanoparticle system and delivered to a target cell or tissue of the present invention may be combined with a diagnostic, antigen, prophylactic or prognostic agents. Any chemical compound to be administered to an individual may be delivered using the adjuvant nanoparticle delivery system of the invention.

In certain embodiments, a lipid to be used in nanoparticle can be, but is not limited to, one or a plurality of the following: phosphatidylcholine, lipid A, cholesterol, dolichol, sphingosine, sphingomyelin, ceramide, glycosylceramide, cerebroside, sulfatide, phytosphingosine, phosphatidyl-ethanolamine, phosphatidylglycerol, phosphatidylinositol, phosphatidylserine, cardiolipin, phosphatidic acid, and lyso-phophatides. In certain embodiments, an immunomodulatory agent can be conjugated to the surface of a nanoparticle. In some embodiments, the nanoparticle surface membrane can be modified with targeting molecules that can selectively deliver the cell death stimulating agent(s), e.g., immunogenic or non-immunogenic cancer cell death stimulating agent, to specific transmembrane expressing cells (e.g., tumor vascular endothelial cells).

### CAR T Cells

In some embodiments, the cell death stimulating agent is a chimeric antigen receptor T cell (CAR T cell).

As used herein, a “chimeric antigen receptor” (CAR) is an artificially constructed hybrid protein or polypeptide comprising a specificity or recognition (i.e. binding) domain linked to an immune receptor responsible for signal transduction in lymphocytes. The binding domain is typically derived from a Fab antibody fragment that has been fashioned into a single chain scFv via the introduction of a flexible linker between the antibody chains within the specificity domain. Other possible specificity domains can include the signaling portions of hormone or cytokine molecules, the extracellular domains of receptors, and peptide ligands or peptides isolated by library (e.g. phage) screening (see Ramos and Dotti, (2011) Expert Opin Bio Ther 11 (7): 855). Flexibility between the signaling and the binding portions of the CAR may be a desirable characteristic to allow for more optimum interaction between the target and the binding domain, so often a hinge region is included. One example of a structure that can be used is the CH2-CH3 region from an immunoglobulin such as an IgG molecule. The signaling domain of the typical CAR comprises intracellular domains of the TCR-CD3 complex such as the zeta chain. Alternatively, the γ chain of an Fe receptor may be used. The transmembrane portion of the typical CAR can comprise transmembrane portions of proteins such as CD4, CD8 or CD28 (Ramos and Dotti, ibid). Characteristics of some CARs include their ability to redirect T-cell specificity and reactivity toward a selected target in a non-MHC-restricted manner. The non-MHC-restricted target recognition gives T-cells expressing CARs the ability to recognize a target independent of antigen processing, thus bypassing a major mechanism of tumor escape.

In some embodiments, the surface of the CAR T cells is decorated with one or more targeting molecules. In some embodiments, the one or more targeting molecules are embedded in the lipid membrane of the CAR T cell. In some embodiments, the one or more targeting molecules are associated with the lipid membrane of the CAR T cell (e.g., binding to molecule on the exterior of the CAR T cell, covalently linked to a molecule on the exterior of the CAR T cell). In some embodiments, there are two or more different types of targeting molecules on the exterior of the CAR T cells.

### Treatment of Disease or Disorders

As used herein, the term “therapeutically effective amount” means an amount of a therapeutic, prophylactic, and/or diagnostic agent (e.g., inventive vaccine nanocarrier) that is sufficient, when administered to a subject suffering from or susceptible to a disease, disorder, and/or condition, to treat, alleviate, ameliorate, relieve, alleviate symptoms of, prevent, delay onset of, inhibit progression of, reduce severity of, and/or reduce incidence of the disease, disorder, and/or condition. The term is also intended to refer to an amount of nanocarrier or composition thereof provided herein that modulates an immune response in a subject.

As used herein, the term “therapeutic agent” refers to any agent that, when administered to a subject, has a therapeutic, prophylactic, and/or diagnostic effect and/elicits a desired biological and/or pharmacological effect.

As used herein, the term “treating” refers to a partially or completely alleviating, ameliorating, relieving, delaying onset of, inhibiting progression of, reducing severity of, and/or reducing incidence of one or more symptoms or features of a particular disease, disorder, and/or condition. For example, “treating” a microbial infection may refer to inhibiting survival, growth, and/or spread of the microbe. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition. In some embodiments, treatment comprises delivery of an inventive vaccine nanocarrier to a subject.

In some embodiments, the disease or disorder is a cancer. In some embodiments, the cancer is a non-immunogenic cancer. In some embodiments, the cancer is a hematological cancer. In some embodiments, the cancer is a solid tumor. In some embodiments, the cancer is melanoma, pancreatic cancer, and colorectal cancer.

In some embodiments, the disease or disorder the cancer is breast cancer, prostate cancer, renal cell carcinoma, bone metastasis, lung cancer or metastasis, osteosarcoma, multiple myeloma, astrocytoma, pilocytic astrocytoma, dysembryoplastic neuroepithelial tumor, oligodendrogliomas, ependymoma, glioblastoma multiforme, mixed gliomas, oligoastrocytomas, medulloblastoma, retinoblastoma, neuroblastoma, germinoma, teratoma, gangliogliomas, gangliocytoma, central gangliocytoma, primitive neuroectodermal tumors (PNET, e.g. medulloblastoma, medulloepithelioma, neuroblastoma, retinoblastoma, ependymoblastoma), tumors of the pineal parenchyma (e.g. pineocytoma, pineoblastoma), ependymal cell tumors, choroid plexus tumors, neuroepithelial tumors of uncertain origin (e.g. gliomatosis cerebri, astroblastoma), esophageal cancer, colorectal cancer, CNS, ovarian, melanoma pancreatic cancer, squamous cell carcinoma, hematologic cancer (e.g., leukemia, lymphoma, and multiple myeloma), colon cancer, rectum cancer, stomach cancer, kidney cancer, pancreas cancer, skin cancer, or a combination thereof.

The term “diagnosis” as used herein refers to methods by which the skilled artisan can estimate and/or determine whether or not a patient is suffering from a given disease or condition. The skilled artisan often makes a diagnosis on the basis of one or more diagnostic indicators, e.g., a biomarker, the presence, absence, amount, or change in amount of which is indicative of the presence, severity, or absence of the condition.

As used herein the term “prognosis” shall be taken to mean an indicator of the predicted progression of the disease (including but not limited to aggressiveness and metastatic potential) and/or predicted patient survival time.

As used herein, the term “identifying” or grammatical variations thereof refer to determining the presence of a diagnostic indicators, e.g., tumor vascular endothelial cells expressing one or more transmembrane molecules from Tables 8-10, wherein the one or more transmembrane molecules are upregulated when compared to a non-diseased control.

The term “control” or “control sample,” as used herein, refers to any clinically relevant control sample, including, for example, a sample from a healthy subject not afflicted with the disease or condition being assayed (e.g., cancer), a sample from a subject having a less severe or slower progressing disease or condition (e.g., cancer) than the subject to be assessed, a sample from a subject having some other type of cancer or disease, and the like. A control sample may include a sample derived from one or more subjects. A control sample may also be a sample made at an earlier timepoint from the subject to be assessed. For example, the control sample could be a sample taken from the subject to be assessed before the onset of the disease or condition being assayed (e.g., cancer), at an earlier stage of disease, or before the administration of treatment or of a portion of treatment. The control sample may also be a sample from an animal model, or from a tissue or cell lines derived from the animal model, of the disease or condition being assayed (e.g., cancer). For example, the expression level of a molecule, such as the proteins listed in Tables 8-10, in a control sample that consists of a group of measurements may be determined based on any appropriate statistical measure, such as, for example, measures of central tendency including average, median, or modal values.

The term “control level” refers to an accepted or pre-determined expression level of a molecule, such as the proteins listed in Tables 8-10 which is used to compare with the expression level of a molecule, such as the proteins listed in Tables 8-10 in a sample derived from a subject. In one embodiment, the control level of a molecule, such as the proteins listed in Tables 8-10 is based on the expression level of the molecule in sample(s) from a subject(s) having slow disease progression. In another embodiment, the control level of the molecule, such as proteins listed in Tables 8-10, is based on the expression level in a sample from a subject(s) having rapid disease progression. In another embodiment, the control level of the molecule, such as proteins listed in Tables 8-10, in based on sample(s) from an unaffected, i.e., non-diseased, subject(s), i.e., a subject who does not have a disease or disorder (e.g., cancer). In yet another embodiment, the control level of the molecule, such as proteins listed in Tables 8-10, is based on the expression level of the molecule in a sample from a subject(s) prior to the administration of a therapy for the disease or disorder (e.g., cancer). In yet another embodiment, the control level of the molecule, such as proteins listed in Tables 8-10, is based on the expression level of the molecule in a sample from a subject(s) after the administration of a therapy for the disease or disorder (e.g., cancer). In one embodiment, the control level of the molecule, such as proteins listed in Tables 8-10, is based on the level in a sample(s) from an animal model of a disease or disorder, (e.g., cancer), a cell, or a cell line derived from the animal model of a disease or disorder, (e.g., cancer).

In some embodiments, the disclosure provides methods for treating a subject having cancer including the use of a composition comprising a targeting molecule. In some embodiments, the targeting molecule binds to transmembrane molecule on a tumor vascular endothelial cell selected from Tables 8-10. In some embodiments, the tumor vascular endothelial cell is a cancer cell and/or is a venular cell. In some embodiments, the transmembrane molecule is upregulated in the tumor vascular endothelial cell when compared to a non-tumor vascular endothelial control cell. In some embodiments, the composition further comprises an agent that induces an agent that induces cell death, e.g., an agent that induces immunogenic or non-immunogenic cancer cell death, in the tumor vascular endothelial cell expressing the target transmembrane molecule.

In some embodiments, the agent that induces an agent that induces an cell death is a chemotherapeutic agent or a CAR T cell. In some embodiments, the agent is a CAR T cell.

In some embodiments, the targeting molecule is an antibody or antigen-binding fragment. In some embodiments, the targeting molecule is a nanobody.

In some embodiments, the targeting molecule is a nucleic acid, e.g., DNA or RNA, e.g., aptamer.

In some embodiments, the methods of use provided herein include identifying if a subject has tumor vascular endothelial cells that express one or more transmembrane molecules from Tables 8-10. In some embodiments, the method comprises administering a targeting molecule coupled to a diagnostic agent and determine a subject has tumor vascular endothelial cells that express one or more transmembrane molecules from Tables 8-10. In some embodiments, the method comprises determining the one or more transmembrane molecules are upregulated in comparison to a control cell. In some embodiments, the method comprises determining the one or more transmembrane molecules are downregulated in comparison to a control cell.

In some embodiments, the methods of use provided herein include determining is a treatment of cancer in a subject is effective. In some embodiments, the targeting molecule is used to determine the presence of tumor vascular endothelial cells expressing the one or more transmembrane molecules before treatment and after treatment and compared to a non-tumor vascular endothelial control cell. In some embodiments, after treatment the presence of tumor vascular endothelial cells expressing one or more transmembrane molecules is decreased in comparison to prior administration of the treatment indicating the treatment is effective. In some embodiments, after treatment the presence of tumor vascular endothelial cells expressing one or more transmembrane molecules is increased or stays the same in comparison to prior administration of the treatment indicating the treatment is not effective.

In some embodiments, the methods of use provided herein include modifying gene expression of one or more transmembrane molecules in a tumor vascular endothelial cell, wherein the one or more transmembrane molecules are upregulated in comparison to a control cell. In some embodiments, the tumor vascular endothelial cell is contacted with a composition comprising a targeting molecule which binds to one or more transmembrane molecules as listed in Tables 8-10 and a nucleic acid capable of modifying the expression of said transmembrane molecule.

In some embodiments, the nucleic acid capable of modifying the expression of the transmembrane molecule encodes an inhibitory RNA molecule or a CRISPR-Cas9 system.

In certain circumstances it will be desirable to deliver the agent that induces cell death, e.g., an agent that induces immunogenic or non-immunogenic cell death, and targeting molecule in suitably formulated compositions disclosed herein either by pipette, retro-orbital injection, subcutaneously, intraocularly, intravitreally, parenterally, subcutaneously, intravenously, intracerebroventricular (ICV), intravenous injection into the cisterna magna (ICM), intracerebro-ventricularly, intramuscularly, intrathecally, intraspinally, orally, intraperitoneally, by oral or nasal inhalation, or by direct application or injection to one or more cells, tissues, or organs.

In some embodiments, the targeting molecule is associated with a nanoparticle comprising nucleic acids capable of altering gene expression in a cell. In some embodiments, the targeting molecule targets a transmembrane molecule on a tumor vascular endothelial cell. In some embodiments, the expression of the transmembrane molecule is upregulated in comparison to expression in a non-tumor vascular endothelial control cell.

As used herein, the term “gene” may include not only coding sequences but also regulatory regions such as promoters, enhancers, and termination regions. The term further can include all introns and other DNA sequences spliced from the mRNA transcript, along with variants resulting from alternative splice sites. The term further refers to a coding sequence for a desired expression product of a polynucleotide sequence such as a polypeptide, peptide, protein or interfering RNA including short interfering RNA (siRNA), miRNA or small hairpin RNA (shRNA). The sequences can also include degenerate codons of a reference sequence or sequences that may be introduced to provide codon preference in a specific organism or cell type. As used herein, the term “heterologous gene” refers to a gene provided to the target cell by an exogenous source, such as a viral vector, e.g., rAAV. In some embodiments, the gene encodes a polypeptide or a nucleic acid molecule, such as microRNA (miRNA), artificial microRNA (amiRNA), and short hairpin RNA (shRNA).

The term “viral vector” refers to a nucleic acid molecule that includes virus-derived nucleic acid elements that facilitate transfer and expression of non-native nucleic acid molecules within a cell. The term adeno-associated viral vector refers to a viral vector or plasmid containing structural and functional genetic elements, or portions thereof, that are primarily derived from AAV. The term “retroviral vector” refers to a viral vector or plasmid containing structural and functional genetic elements, or portions thereof, that are primarily derived from a retrovirus. The term “lentiviral vector” refers to a viral vector or plasmid containing structural and functional genetic elements, or portions thereof, that are primarily derived from a lentivirus, and so on. The term “hybrid vector” refers to a vector including structural and/or functional genetic elements from more than one virus type.

As used herein, the term “adenovirus vector” refers to those constructs containing adenovirus sequences sufficient to (a) support packaging of an expression construct and (b) to express a coding sequence that has been cloned therein in a sense or antisense orientation. A recombinant Adenovirus vector includes a genetically engineered form of an adenovirus. Knowledge of the genetic organization of adenovirus, a 36 kb, linear, double-stranded DNA virus, allows substitution of large pieces of adenoviral DNA with foreign sequences up to 7 kb. In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification. As used herein, the term “AAV vector” in the context of the present invention includes without limitation AAV type 1, AAV type 2, AAV type 3 (including types 3A and 3B), AAV type 4, AAV type 5, AAV type 6, AAV type 7, AAV type 8, AAV type 9, AAV type 10, AAV type 11, avian AAV, bovine AAV, canine AAV, equine AAV, and ovine AAV and any other AAV now known or later discovered. See, e.g., BERNARD N. FIELDS et al., VIROLOGY, volume 2, chapter 69 (4th ed., Lippincott-Raven Publishers). A number of additional AAV serotypes and clades have been identified (see, e.g., Gao et al., (2004) J. Virol. 78:6381-6388 and Table 1), which are also encompassed by the term “AAV.” Adenovirus is particularly suitable for use as a gene transfer vector because of its mid-sized genome, ease of manipulation, high titer, wide target-cell range, and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression, and host cell shut-off. The products of the late genes, including the majority of the viral capsid proteins, are expressed only after significant processing of a single primary transcript issued by the major late promoter (MLP). The MLP is particularly efficient during the late phase of infection, and all the mRNAs issued from this promoter possess a 5′-tripartite leader (TPL) sequence which makes them preferred mRNAs for translation.

Other than the requirement that an adenovirus vector be replication defective, or at least conditionally defective, the nature of the adenovirus vector is not believed to be crucial to the successful practice of particular embodiments disclosed herein. The adenovirus may be of any of the 42 different known serotypes or subgroups A-F. In some embodiments, adenovirus type 5 of subgroup C is the preferred starting material in order to obtain a conditional replication-defective adenovirus vector for use in some embodiments, since Adenovirus type 5 is a human adenovirus about which a great deal of biochemical and genetic information is known, and it has historically been used for most constructions employing adenovirus as a vector.

As indicated, the typical vector is replication defective and will not have an adenovirus E1 region. Thus, it will be most convenient to introduce the polynucleotide encoding the gene of interest at the position from which the E1-coding sequences have been removed. However, the position of insertion of the construct within the adenovirus sequences is not critical. The polynucleotide encoding the gene of interest may also be inserted in lieu of a deleted E3 region in E3 replacement vectors or in the E4 region where a helper cell line or helper virus complements the E4 defect.

Adeno-Associated Virus (AAV) is a parvovirus, discovered as a contamination of adenoviral stocks. It is a ubiquitous virus (antibodies are present in 85% of the US human population) that has not been linked to any disease. It is also classified as a dependovirus, because its replication is dependent on the presence of a helper virus, such as adenovirus. Various serotypes have been isolated, of which AAV-2 is the best characterized. AAV has a single-stranded linear DNA that is encapsidated into capsid proteins VP1, VP2 and VP3 to form an icosahedral virion of 20 to 24 nm in diameter.

The AAV DNA is 4.7 kilobases long. It contains two open reading frames and is flanked by two ITRs. There are two major genes in the AAV genome: rep and cap. The rep gene codes for proteins responsible for viral replications, whereas cap codes for capsid protein VP1-3. Each ITR forms a T-shaped hairpin structure. These terminal repeats are the only essential cis components of the AAV for chromosomal integration. Therefore, the AAV can be used as a vector with all viral coding sequences removed and replaced by the cassette of genes for delivery. Three AAV viral promoters have been identified and named p5, p19, and p40, according to their map position. Transcription from p5 and p19 results in production of rep proteins, and transcription from p40 produces the capsid proteins.

AAVs stand out for use within the current disclosure because of their superb safety profile and because their capsids and genomes can be tailored to allow expression in selected cell populations. scAAV refers to a self-complementary AAV. pAAV refers to a plasmid adeno-associated virus. rAAV refers to a recombinant adeno-associated virus.

Other viral vectors may also be employed. For example, vectors derived from viruses such as vaccinia virus, polioviruses and herpes viruses may be employed. They offer several attractive features for various mammalian cells.

Retrovirus. Retroviruses are a common tool for gene delivery. “Retrovirus” refers to an RNA virus that reverse transcribes its genomic RNA into a linear double-stranded DNA copy and subsequently covalently integrates its genomic DNA into a host genome. Once the virus is integrated into the host genome, it is referred to as a “provirus.” The provirus serves as a template for RNA polymerase II and directs the expression of RNA molecules which encode the structural proteins and enzymes needed to produce new viral particles.

Illustrative retroviruses suitable for use in some embodiments include: Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV) and Rous Sarcoma Virus (RSV) and lentivirus.

“Lentivirus” refers to a group (or genus) of complex retroviruses. Illustrative lentiviruses include: HIV (human immunodeficiency virus; including HIV type 1, and HIV type 2); visna-maedi virus (VMV); the caprine arthritis-encephalitis virus (CAEV); equine infectious anemia virus (EIAV); feline immunodeficiency virus (FIV); bovine immune deficiency virus (BIV); and simian immunodeficiency virus (SIV). In some embodiments, HIV based vector backbones (i.e., HIV cis-acting sequence elements) can be used.

A safety enhancement for the use of some vectors can be provided by replacing the U3 region of the 5′ LTR with a heterologous promoter to drive transcription of the viral genome during production of viral particles. Examples of heterologous promoters which can be used for this purpose include, for example, viral simian virus 40 (SV40) (e.g., early or late), cytomegalovirus (CMV) (e.g., immediate early), Moloney murine leukemia virus (MoMLV), Rous sarcoma virus (RSV), and herpes simplex virus (HSV) (thymidine kinase) promoters. Typical promoters are able to drive high levels of transcription in a Tat-independent manner. This replacement reduces the possibility of recombination to generate replication-competent virus because there is no complete U3 sequence in the virus production system. In some embodiments, the heterologous promoter has additional advantages in controlling the manner in which the viral genome is transcribed. For example, the heterologous promoter can be inducible, such that transcription of all or part of the viral genome will occur only when the induction factors are present. Induction factors include one or more chemical compounds or the physiological conditions such as temperature or pH, in which the host cells are cultured.

In some embodiments, expression of heterologous sequences in viral vectors is increased by incorporating posttranscriptional regulatory elements, efficient polyadenylation sites, and optionally, transcription termination signals into the vectors. A variety of posttranscriptional regulatory elements can increase expression of a heterologous nucleic acid. Examples include the woodchuck hepatitis virus posttranscriptional regulatory element (WPRE; Zufferey et al, 1999, J. Virol., 73:2886); the posttranscriptional regulatory element present in hepatitis B virus (HPRE) (Smith et al., Nucleic Acids Res. 26 (21): 4818-4827, 1998); and the like (Liu et al., 1995, Genes Dev., 9:1766). In some embodiments, vectors include a posttranscriptional regulatory element such as a WPRE or HPRE. In some embodiments, vectors lack or do not include a posttranscriptional regulatory element such as a WPRE or HPRE.

Elements directing the efficient termination and polyadenylation of a heterologous nucleic acid transcript can increase heterologous gene expression. Transcription termination signals are generally found downstream of the polyadenylation signal. In some embodiments, vectors include a polyadenylation sequence 3′ of a polynucleotide encoding a molecule (e.g., protein) to be expressed. The term “poly(A) site” or “poly(A) sequence” denotes a DNA sequence which directs both the termination and polyadenylation of the nascent RNA transcript by RNA polymerase II. Polyadenylation sequences can promote mRNA stability by addition of a poly(A) tail to the 3′ end of the coding sequence and thus, contribute to increased translational efficiency. Particular embodiments may utilize BGHpA or SV40 pA. In some embodiments, a preferred embodiment of an expression construct includes a terminator element. These elements can serve to enhance transcript levels and to minimize read through from the construct into other plasmid sequences.

In some embodiments, a viral vector further includes one or more insulator elements. Insulator elements may contribute to protecting viral vector-expressed sequences, e.g., effector elements or expressible elements, from integration site effects, which may be mediated by as acting elements present in genomic DNA and lead to deregulated expression of transferred sequences (i.e., position effect; see, e.g., Burgess-Beusse et al, PNAS., USA, 99:16433, 2002; and Zhan et al., Hum. Genet., 109:471, 2001). In some embodiments, viral transfer vectors include one or more insulator elements at the 3′ LTR and upon integration of the provirus into the host genome, the provirus includes the one or more insulators at both the 5′ LTR and 3′ LTR, by virtue of duplicating the 3′ LTR. Suitable insulators for use in particular embodiments include the chicken b-globin insulator (see Chung et al., Cell 74:505, 1993; Chung et al., PNAS USA 94:575, 1997; and Bell et al., Cell 98:387, 1999), SP10 insulator (Abhyankar et al., JBC 282:36143, 2007), or other small CTCF recognition sequences that function as enhancer blocking insulators (Liu et al., Nature Biotechnology, 33:198, 2015).

Beyond the foregoing description, a wide range of suitable expression vector types will be known to a person of ordinary skill in the art. These can include commercially available expression vectors designed for general recombinant procedures, for example plasmids that contain one or more reporter genes and regulatory elements required for expression of the reporter gene in cells. Numerous vectors are commercially available, e.g., from Invitrogen, Stratagene, Clontech, etc., and are described in numerous associated guides. In some embodiments, suitable expression vectors include any plasmid, cosmid or phage construct that is capable of supporting expression of encoded genes in mammalian cell, such as pUC or Bluescript plasmid series.

In some embodiments, vectors (e.g., AAV) include AAV9 (Gombash et al., Front Mol Neurosci. 2014; 7:81), AAVrh.10 (Yang, et al., Mol Ther. 2014; 22 (7): 1299-1309), AAV1 R6, AAV1 R7 (Albright et al., Mol Ther. 2018; 26 (2): 510), rAAVrh.8 (Yang, et al., supra), AAV-BR1 (Marchio et al., EMBO Mol Med. 2016; 8 (6): 592), AAV-PHP.S (Chan et al., Nat Neurosci. 2017; 20(8): 1 172), AAV-PHP.B (Deverman et al., Nat Biotechnol. 2016; 34 (2): 204), and AAV-PPS (Chen et al., Nat Med. 2009; 15:1215). The PHP.eB capsid differs from AAV9 such that, using AAV9 as a reference, the sequence DGTLAVPFK (SEQ ID NO: 41) is inserted between amino acids residues 586 and 587 of AAV9.

In some embodiments, AAV comprises AAV type 1 (AAV1), AAV type 2 (AAV2), AAV type 3 (including types AAV3A and AAV3B), AAV type 4 (AAV4), AAV type 5 (AAV5), AAV type 6 (AAV6), AAV type 7 (AAV7), AAV type 8 (AAV8), AAV type 9 (AAV9), AAV type 10 (AAV10), and AAV type 11 (AAV11) and any other AAV now known or later discovered.

### Formulations

Artificial expression constructs and vectors of the present disclosure (referred to herein as physiologically active components) can be formulated with a carrier that is suitable for administration to a cell, tissue slice, animal (e.g., mouse, non-human primate), or human. Physiologically active components within compositions described herein can be prepared in neutral forms, as freebases, or as pharmacologically acceptable salts.

Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Carriers of physiologically active components can include solvents, dispersion media, vehicles, coatings, diluents, isotonic and absorption delaying agents, buffers, solutions, suspensions, colloids, and the like. The use of such carriers for physiologically active components is well known in the art. Except insofar as any conventional media or agent is incompatible with the physiologically active components, it can be used with compositions as described herein.

The phrase “pharmaceutically-acceptable carriers” refer to carriers that do not produce an allergic or similar untoward reaction when administered to a human, and in some embodiments, when administered intravenously (e.g., at the retro-orbital plexus).

In some embodiments, compositions can be formulated for intravenous, intraocular, intravitreal, parenteral, subcutaneous, intracerebro-ventricular, intramuscular, intracerebroventricular, intravenous injection into the cisterna magna (ICM), intrathecal, intraspinal, oral, intraperitoneal, oral or nasal inhalation, or by direct injection in or application to one or more cells, tissues, or organs.

Compositions may include liposomes, lipids, lipid complexes, microspheres, microparticles, nanospheres, and/or nanoparticles.

As used herein, the term “lipid nanoparticle” refers to a vesicle formed by one or more lipid components. Lipid nanoparticles are typically used as carriers for nucleic acid delivery in the context of pharmaceutical development. They work by fusing with a cellular membrane and repositioning its lipid structure to deliver a drug or active pharmaceutical ingredient (API). Generally, lipid nanoparticle compositions for such delivery are composed of synthetic ionizable or cationic lipids, phospholipids (especially compounds having a phosphatidylcholine group), cholesterol, and a polyethylene glycol (PEG) lipid; however, these compositions may also include other lipids. The sum composition of lipids typically dictates the surface characteristics and thus the protein (opsonization) content in biological systems thus driving biodistribution and cell uptake properties.

As used herein, the “liposome” refers to lipid molecules assembled in a spherical configuration encapsulating an interior aqueous volume that is segregated from an aqueous exterior. Liposomes are vesicles that possess at least one lipid bilayer. Liposomes are typical used as carriers for drug/therapeutic delivery in the context of pharmaceutical development. They work by fusing with a cellular membrane and repositioning its lipid structure to deliver a drug or active pharmaceutical ingredient. Liposome compositions for such delivery are typically composed of phospholipids, especially compounds having a phosphatidylcholine group, however these compositions may also include other lipids.

As used herein, the term “ionizable lipid” refers to lipids having at least one protonatable or deprotonatable group, such that the lipid is positively charged at a pH at or below physiological pH (e.g., pH 7.4), and neutral at a second pH, preferably at or above physiological pH. It will be understood by one of ordinary skill in the art that the addition or removal of protons as a function of pH is an equilibrium process, and that the reference to a charged or a neutral lipid refers to the nature of the predominant species and does not require that all of the lipid be present in the charged or neutral form. Generally, ionizable lipids have a pKa of the protonatable group in the range of about 4 to about 7. Ionizable lipids are also referred to as cationic lipids herein.

As used herein, the term “non-cationic lipid” refers to any amphipathic lipid as well as any other neutral lipid or anionic lipid. Accordingly, the non-cationic lipid can be a neutral uncharged, zwitterionic, or anionic lipid.

As used herein, the term “conjugated lipid” refers to a lipid molecule conjugated with a non-lipid molecule, such as a PEG, polyoxazoline, polyamide, or polymer (e g., cationic polymer).

As used herein, the term “excipient” refers to pharmacologically inactive ingredients that are included in a formulation with the API, e.g., ceDNA and/or lipid nanoparticles to bulk up and/or stabilize the formulation when producing a dosage form. General categories of excipients include, for example, bulking agents, fillers, diluents, antiadherents, binders, coatings, disintegrants, flavours, colors, lubricants, glidants, sorbents, preservatives, sweeteners, and products used for facilitating drug absorption or solubility or for other pharmacokinetic considerations.

The formation and use of liposomes is generally known to those of skill in the art. Liposomes have been developed with improved serum stability and circulation half-times (see, for instance, U.S. Pat. No. 5,741,516). Further, various methods of liposome and liposome like preparations as potential drug carriers have been described (see, for instance U.S. Pat. Nos. 5,567,434; 5,552,157; 5,565,213; 5,738,868; and 5,795,587).

The disclosure also provides for pharmaceutically acceptable nanocapsule formulations of the physiologically active components. Nanocapsules can generally entrap compounds in a stable and reproducible way (Quintanar-Guerrero et al., Drug Dev Ind Pharm 24 (12): 11 13-1 128, 1998; Quintanar-Guerrero et al, Pharm Res. 15 (7): 1056-1062, 1998; Quintanar-Guerrero et al., J. Microencapsul. 15 (1): 107-1 19, 1998; Douglas et al, Crit Rev Ther Drug Carrier Syst 3 (3): 233-261, 1987). To avoid side effects due to intracellular polymeric overloading, such ultrafine particles can be designed using polymers able to be degraded in vivo. Biodegradable polyalkyl-cyanoacrylate nanoparticles that meet these requirements are contemplated for use in the present disclosure. Such particles can be easily made, as described in Couvreur et al., J Pharm Sci 69 (2): 199-202, 1980; Couvreur et al., Crit Rev Ther Drug Carrier Syst. 5 (1) 1-20, 1988; zur Muhlen et al., Eur J Pharm Biopharm, 45 (2): 149-155, 1998; Zambau x et al., J Control Release 50 (1-3): 31-40, 1998; and U.S. Pat. No. 5,145,684.

Injectable compositions can include sterile aqueous solutions or dispersions and sterile powders for the extemporaneous preparation of sterile injectable solutions or dispersions (U.S. Pat. No. 5,466,468). For delivery via injection, the form is sterile and fluid to the extent that it can be delivered by syringe. In some embodiments, it is stable under the conditions of manufacture and storage, and optionally contains one or more preservative compounds against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion, and/or by the use of surfactants. The prevention of the action of microorganisms can be brought about by various antibacterial and/or antifungal agents, for example, parabens, chlorobutanol, phenol, sorbic acid, thimerosal, and the like. In various embodiments, the preparation will include an isotonic agent(s), for example, sugar(s) or sodium chloride. Prolonged absorption of the injectable compositions can be accomplished by including in the compositions of agents that delay absorption, for example, aluminum monostearate and gelatin. Injectable compositions can be suitably buffered, if necessary, and the liquid diluent first rendered isotonic with sufficient saline or glucose.

Dispersions may also be prepared in glycerol, liquid polyethylene glycols, and mixtures thereof and in oils. As indicated, under ordinary conditions of storage and use, these preparations can contain a preservative to prevent the growth of microorganisms.

Sterile compositions can be prepared by incorporating the physiologically active component in an appropriate amount of a solvent with other optional ingredients (e.g., as enumerated above), followed by filtered sterilization. Generally, dispersions are prepared by incorporating the various sterilized physiologically active components into a sterile vehicle that contains the basic dispersion medium and the required other ingredients (e.g., from those enumerated above). In the case of sterile powders for the preparation of sterile injectable solutions, preferred methods of preparation can be vacuum-drying and freeze-drying techniques which yield a powder of the physiologically active components plus any additional desired ingredient from a previously sterile-filtered solution thereof.

Oral compositions may be in liquid form, for example, as solutions, syrups or suspensions, or may be presented as a drug product for reconstitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid). The compositions may take the form of, for example, tablets or capsules prepared by conventional means with pharmaceutically acceptable excipients such as binding agents (e.g., pregelatinized maize starch, polyvinyl pyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). Tablets may be coated by methods well-known in the art.

Inhalable compositions can be delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

Compositions can also include microchip devices (U.S. Pat. No. 5,797,898), ophthalmic formulations (Bourlais et al, Prog Retin Eye Res, 17 (1): 33-58, 1998), transdermal matrices (U.S. Pat. Nos. 5,770,219 and 5,783,208) and feedback-controlled delivery (U.S. Pat. No. 5,697,899).

Supplementary active ingredients can also be incorporated into the compositions.

Typically, compositions can include at least 0.1% of the physiologically active components or more, although the percentage of the physiologically active components may, of course, be varied and may conveniently be between 1 or 2% and 70% or 80% or more or 0.5-99% of the weight or volume of the total composition. Naturally, the amount of physiologically active components in each physiologically-useful composition may be prepared in such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of compositions and dosages may be desirable.

In some embodiments, for administration to humans, compositions should meet sterility, pyrogenicity, and the general safety and purity standards as required by United States Food and Drug Administration (FDA) or other applicable regulatory agencies in other countries.

### EXAMPLES

### Example 1: Determining Expression of Genes in Tumor Vascular Endothelial Cells

A hallmark of solid tumors is the formation of new vasculature (angiogenesis). This process is required to support tumor growths beyond a few millimeters in size due to the limit of oxygen and nutrient diffusion within neoplastic tissues (Folkman, J. Tumor angiogenesis: therapeutic implications. N Engl J Med, 285, 1182-1186, (1971)). Tumor neovasculature is often poorly adhesive for blood-borne T cells, which is thought to present a major impediment to T cell dependent immunotherapy (Peske J D, Woods A B, Engelhard V H. Control of CD8 T-Cell Infiltration into Tumors by Vasculature and Microenvironment. Adv Cancer Res., 128, 263-307, (2015)). In order to gain a better understanding of this issue, normal microvasculature most be analyzed, which consists of a network of functionally specialized vessels, including arteries, arterioles, venules and veins, which are all connected by a common capillary network.

Arteries and arterioles regulate blood flow, while gas and nutrient exchange takes place at the capillary level (Potente, M., Mäkinen, T. Vascular heterogeneity and specialization in development and disease. Nat Rev Mol Cell Biol 18, 477-494 (2017)). Using intravital microscopy, it has been shown that the recruitment of blood-borne leukocytes is invariably restricted to postcapillary and collecting venules, whereas capillaries and arterioles do not support leukocyte adhesion (Halin, C., J. Rodrigo Mora, C. Sumen, and U. H. von Andrian. In vivo imaging of lymphocyte trafficking. Annu Rev Cell Dev Biol, 21, 581-603, (2005)). There is strong evidence suggesting that this functional distinction among microvessels is due to segmental specialization of endothelial cells (ECs), not hemodynamic differences (Ley, K., and P. Gaehtgens. Endothelial, not hemodynamic, differences are responsible for preferential leukocyte rolling in rat mesenteric venules. Circ Res, 69, 1034-1041, (1991)). Indeed, microvascular specialization is already apparent during embryogenesis before the initiation of blood flow (Lawson, N. D., and B. M. Weinstein. Arteries and veins: making a difference with zebrafish. Nat Rev Genet, 3, 674-682, (2002)).

Regardless of whether uninflamed microvessels that constitutively recruit leukocytes or acutely or chronically inflamed peripheral tissues are assessed, venules are the exclusive port of exit for blood-borne leukocytes that access the extravascular compartment. Accordingly, multiple studies have shown that most leukocyte adhesion receptors are restricted to venular ECs (VECs), although the expression of these molecules is not uniform in different vascular beds (von Andrian, U. H., and C. R. Mackay. 2000. T-cell function and migration. Two sides of the same coin. N. Engl. J. Med., 343, 1020-1034, (2000)). Several molecules have been identified that specify the differentiation of blood ECs and lymphatic ECs (LECs) and contribute to EC proliferation in tumors, (Rocha, S. F., and R. H. Adams. Molecular differentiation and specialization of vascular beds. Angiogenesis, 12, 139-147, (2009); Oliver, G., and R. S. Srinivasan. Endothelial cell plasticity: how to become and remain a lymphatic endothelial cell. Development, 137, 363-372, (2010)), but the mechanism(s) that render(s) VECs uniquely capable of supporting leukocyte trafficking remain(s) a mystery. A monoclonal antibody (mAb) against DARC (ACKR1) was developed, which selectively recognizes VECs in normal murine tissues (Thiriot, A. et al. Differential DARC/ACKR1 expression distinguishes venular from non-venular endothelial cells in murine tissues, both tumor and non-tumor tissues. BMC Biology, 15, 45, (2017)). In preliminary experiments, using this mAb, as well as a commercial mAb against human DARC, primary VECs, non-venular ECs (NVECs) and LECs were isolated from a variety of murine and human non-malignant tissues to compare EC subsets at the transcriptome and proteome level.

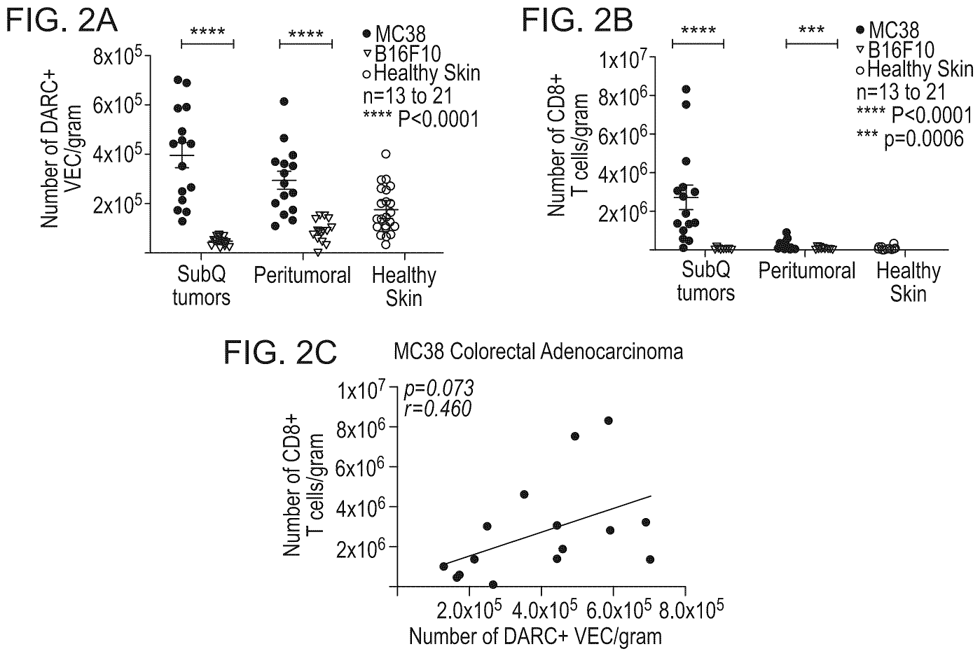

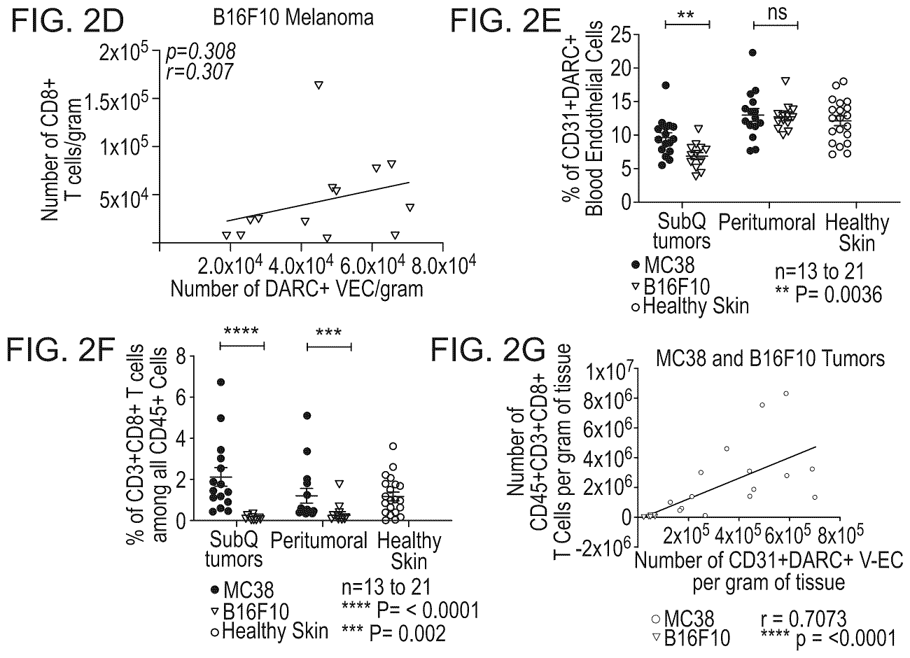

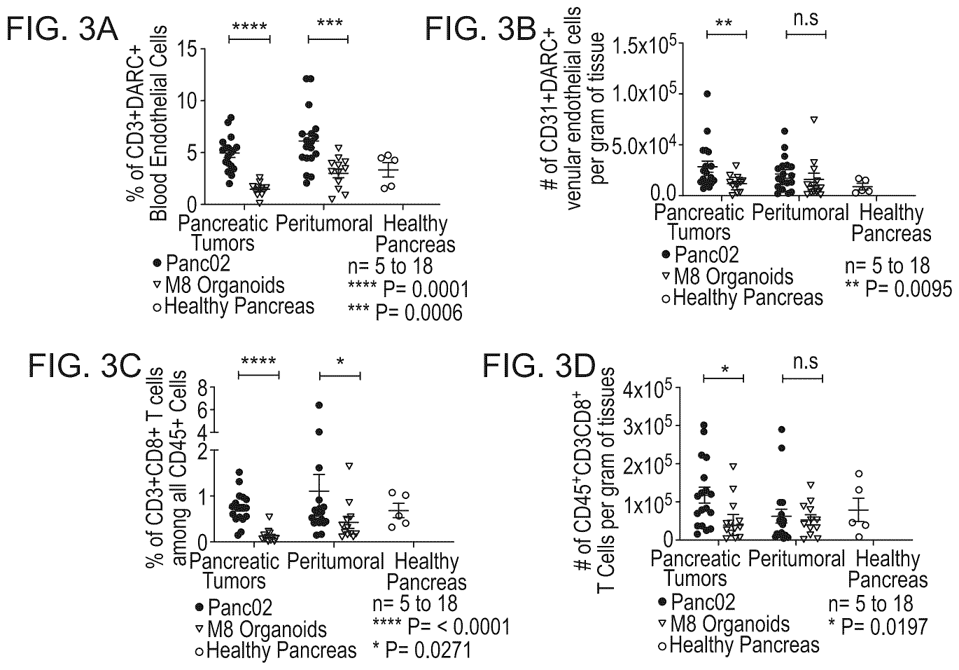

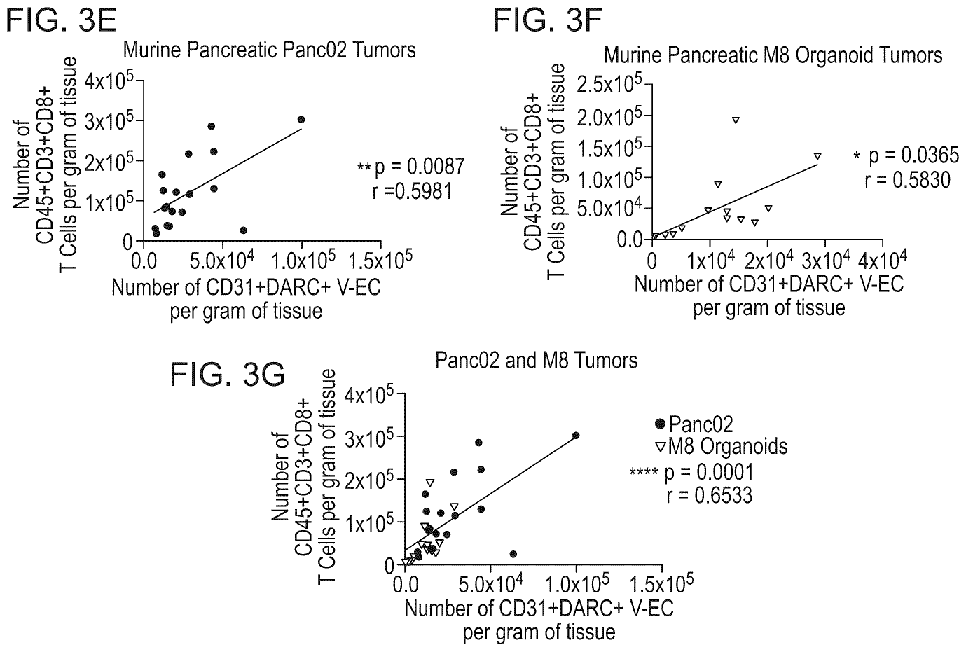

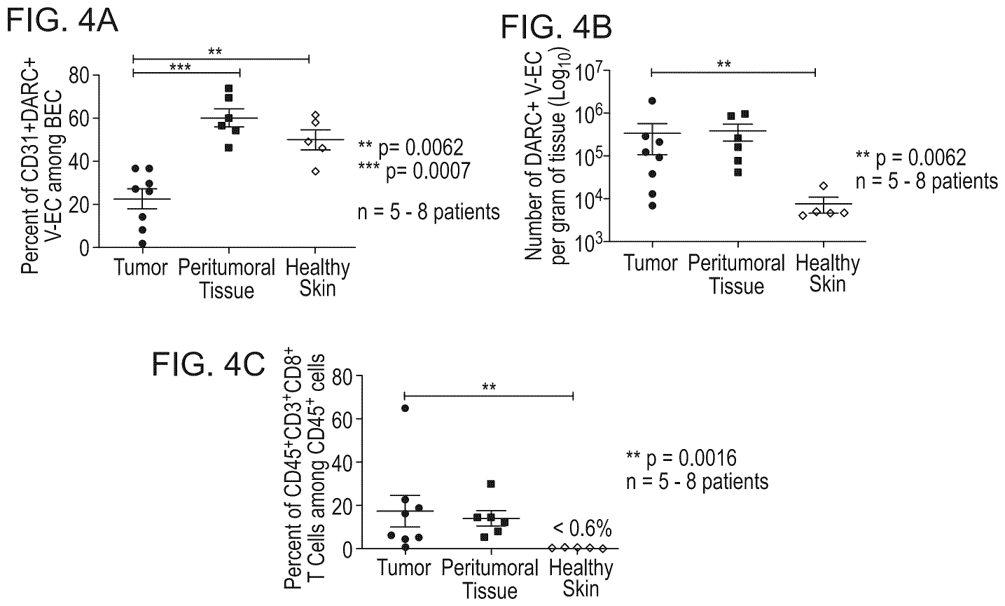

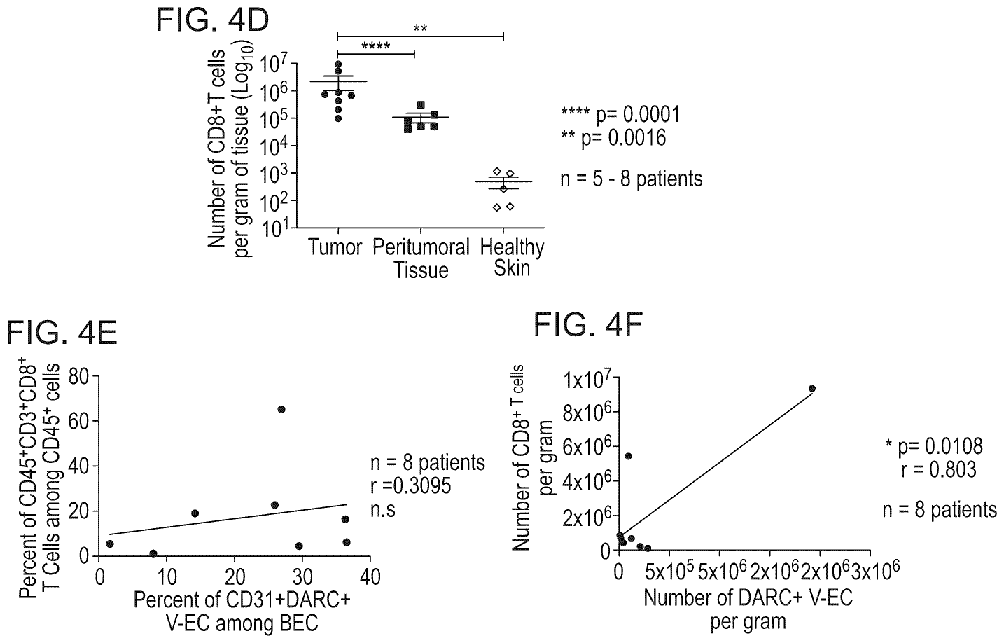

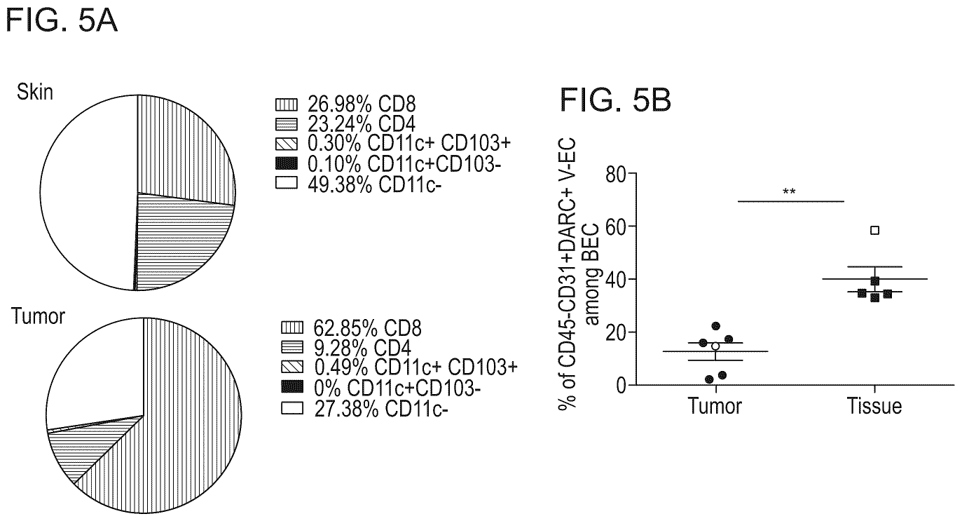

Their analysis revealed that DARC is exquisitely restricted to post-capillary and small collecting venules and completely absent from arteries, arterioles, capillaries, veins, and most lymphatic ECs in every tissue analyzed. Accordingly, intravital microscopy showed that adhesive leukocyte-endothelial interactions were restricted to DARC+ venules. DARC was detectable over the entire circumference of VECs but was more concentrated at cell-cell junctions (Thiriot, A. et al. BMC Biology, 15, 45, (2017) FIG. 2). Analysis of single-cell suspensions suggested that the frequency of VECs among the total microvascular EC pool varies considerably between different tissues.

However, preliminary findings showed that some solid tumors may contain up to 70% fewer venules than healthy tissue, which could explain the paucity of T cells in many cancers. The VECs in those tumors lacked many venular markers, suggesting that newly formed tumor microvessels may not initiate venular differentiation programs. Since VEC are the principal gatekeepers for leukocyte emigration, drugs that promote VEC differentiation could potentially boost tumor infiltration by T cells and thus enhance immunotherapy. Therefore, the hypothesis is that the neovasculature of solid tumors may be inherently suboptimal at recruiting T cells because of inadequate endothelial differentiation into functional venular type microvessels. The transcriptome and proteome of VECs in healthy and in immunogenic vs non immunogenic tumor samples were analyzed to gain a better understanding of the VEC differentiation program.

### Results

Microvascular ECs likely differ between tumors that are susceptible to immunotherapy compared to those that are resistant to treatment; thus the microvascular composition of immunogenic and non-immunogenic tumor models in mouse was characterized. For this purpose, B16 melanoma cells (B16) and MC38 colorectal adenocarcinoma cells (MC38) were used. MC38 has been reported to be susceptible to immunotherapy while B16 melanoma is non-responsive to checkpoint inhibitors. To characterize VEC and T cell content in those tumors, histology and flow cytometry were used to investigate whether tumoral VEC frequency correlates with the number of tumor-infiltrating T cells.

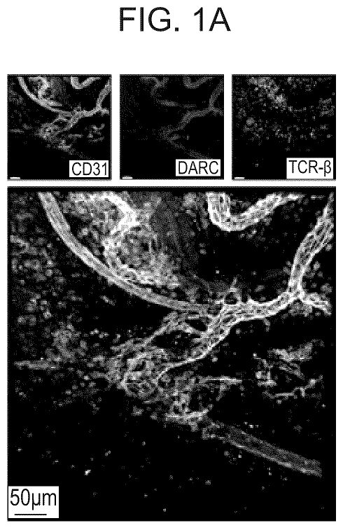

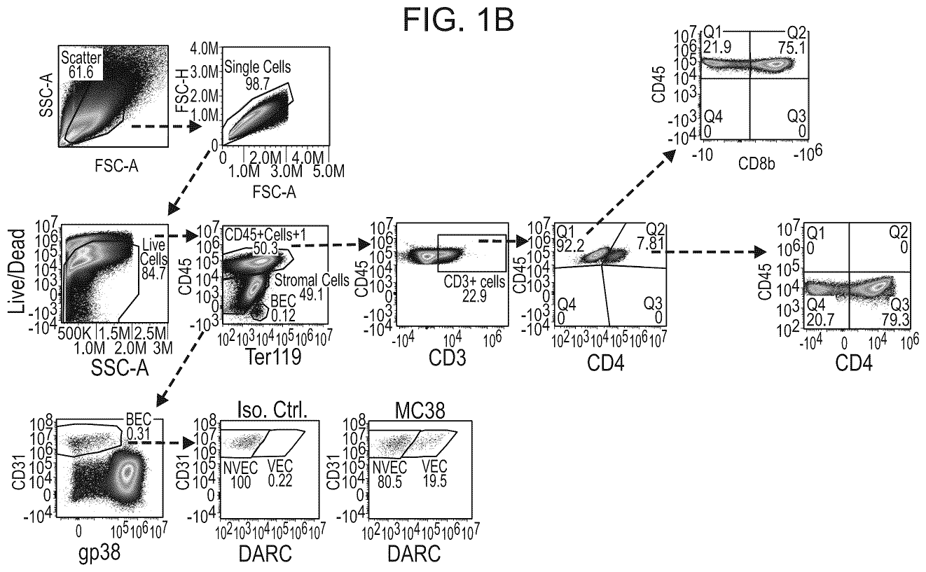

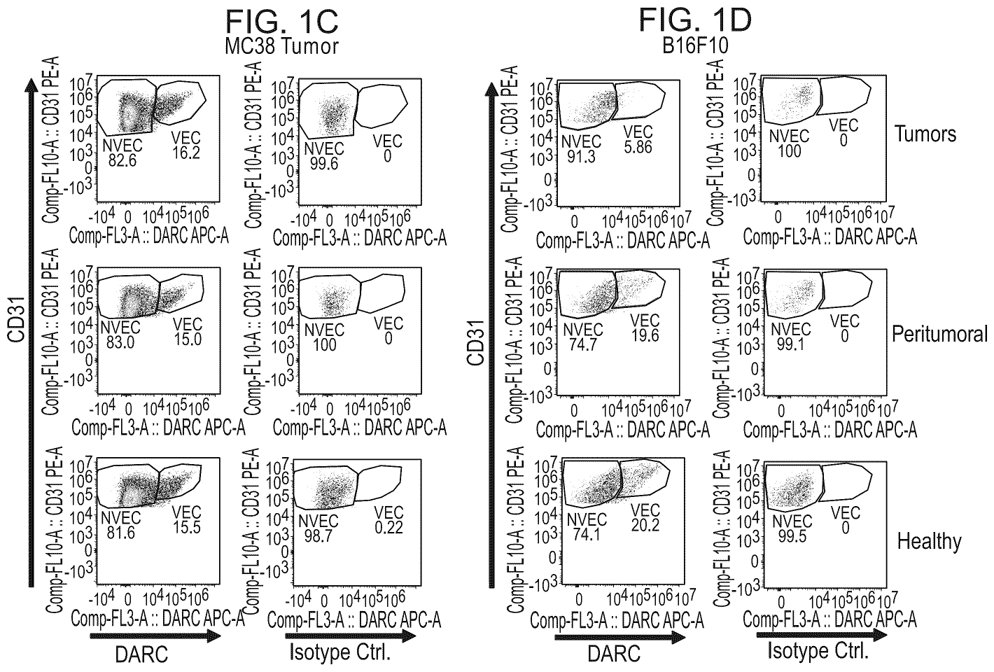

Using antibodies against CD31 (a pan-endothelial marker) and against DARC venules in subcutaneous MC38 implanted in mice were imaged (FIG. 1A). By combining this with T cell imaging through a TCR-β antibody, the proximity between VEC and T cells as well as the extent of the T cell infiltration in this immunogenic tumor model were revealed. This was confirmed by quantification of the T cell infiltrate in both B16 and MC38.

FACS was used to analyze both CD8+ T cells and VEC from B16 and MC38 subcutaneous tumors (gating strategies in FIG. 2) and to quantify how many CD8+ T cells and VEC were observed per gram of tissue in each case. This was repeated for peritumoral tissue and healthy mouse skin (control). A very clear difference in the amount of both VEC and CD8+ T cells in the immunogenic (MC38) vs non-immunogenic model (B16) was observed. The amount of VEC in MC38 was quadrupled compared to what was observed in B16 (FIG. 2A). More strikingly B16 contains very few T cells whereas MC38 contains 3.106 T cells per gram of tissue (FIG. 2B). Interestingly MC38 also showed an increase in VEC and T cell numbers when compared to healthy tissue which confirmed its increased immunogenicity and capacity to attract T cells. Both MC38 and B16 peritumoral tissue showed numbers reflecting an intermediate state between tumor and healthy, as expected.

Next, the direct correlation between VEC and CD8+ T cell numbers in individual animals transplanted with either tumor model was analyzed (FIGS. 2C and 2D). Both models showed a strong correlation between VEC and CD8+ T cells numbers in the tumor confirming that the presence of VEC is likely to increase T cell infiltration. However, the absolute number of CD8+ T cells and VECs is lower in B16 reflecting the increased immunogenicity of MC38.

In order to confirm that the data observed in mouse tumor models held up in human tumor, resections of tumor samples from patients were acquired. Samples of melanoma and pancreatic tumor and healthy control samples (e.g., healthy skin and non-malignant pancreas, respectively) were used.

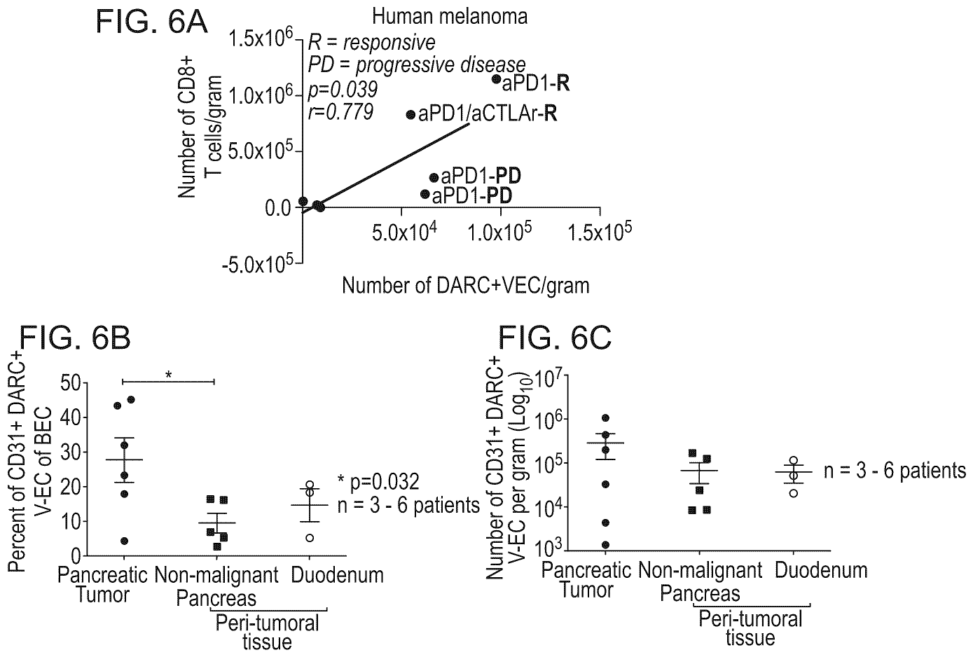

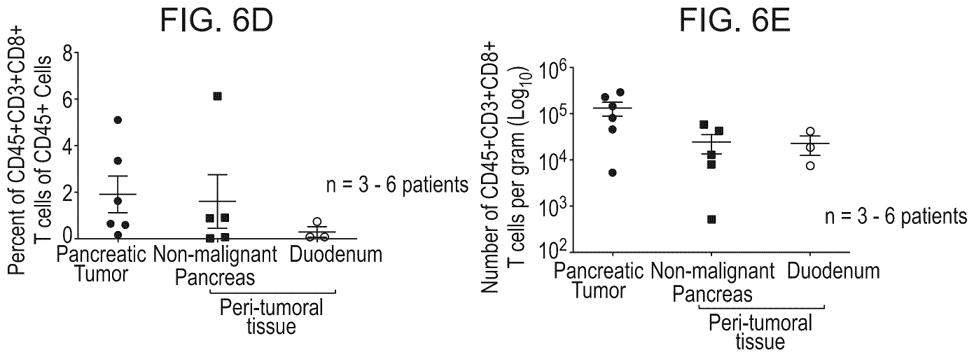

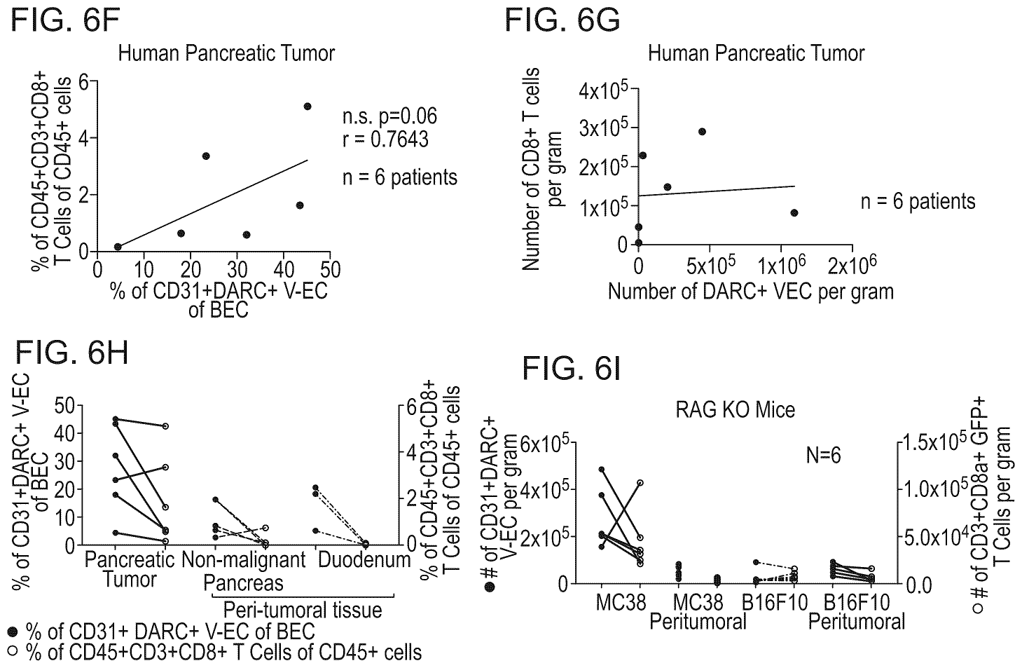

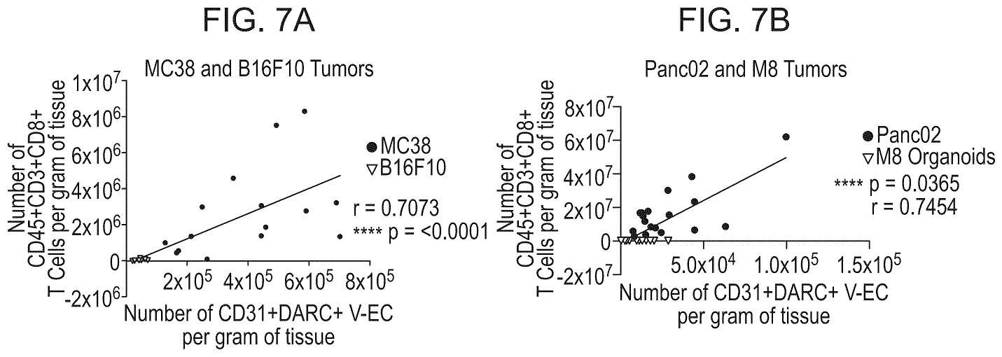

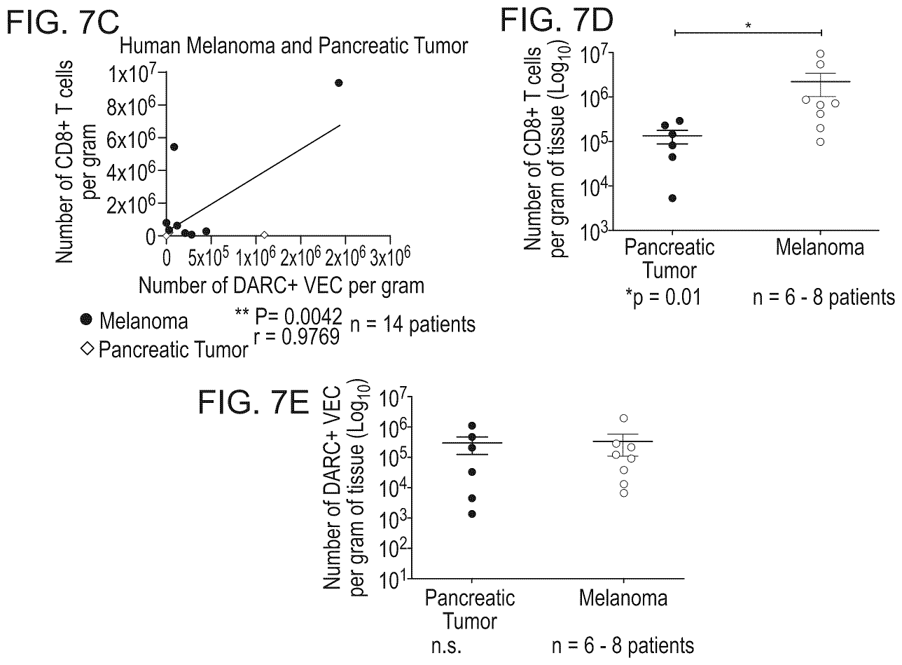

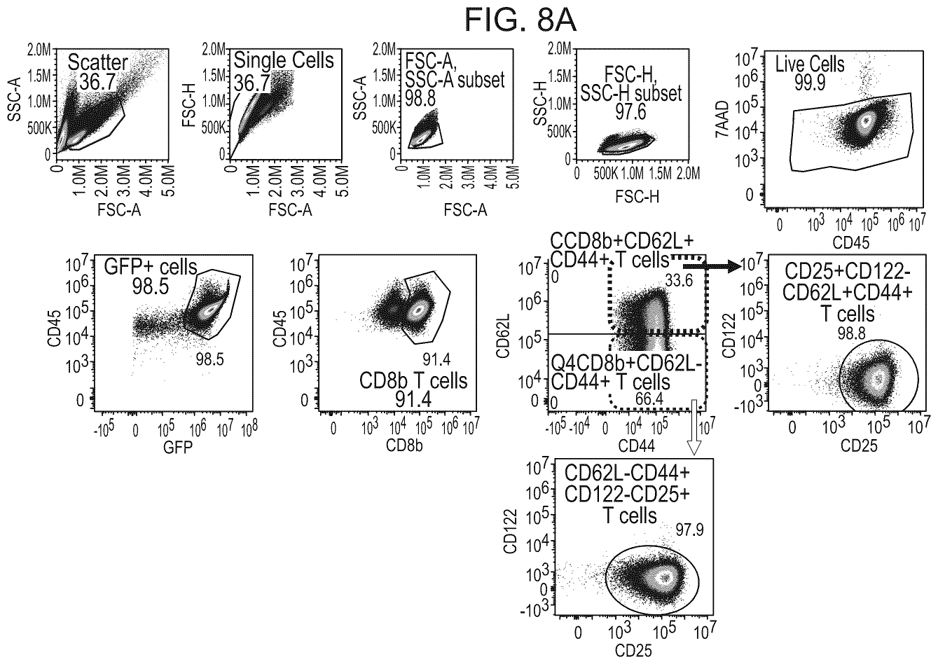

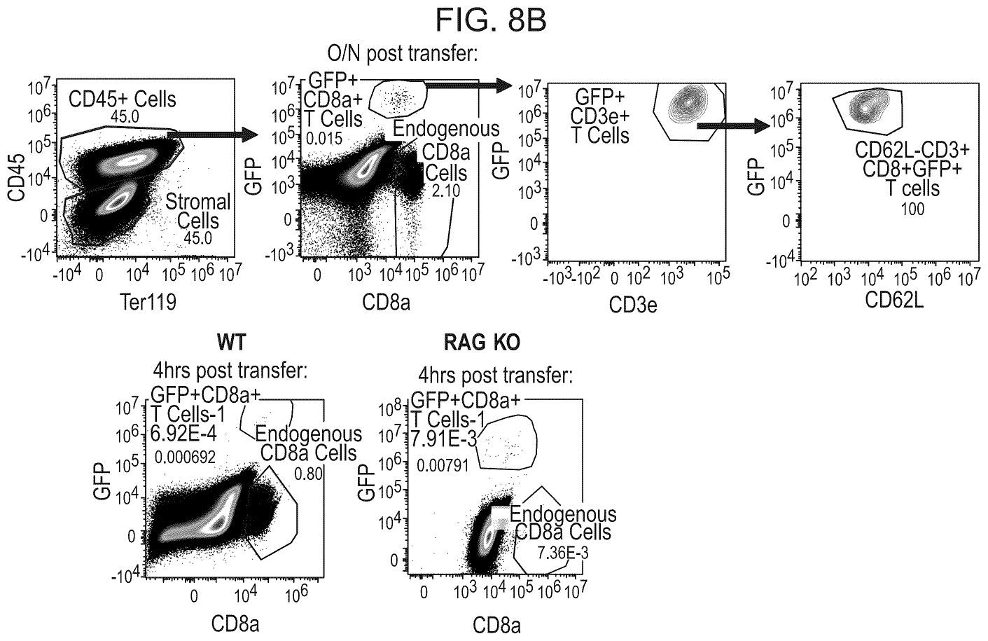

For the melanoma samples, resections from patients who were either responsive or unresponsive (progressive disease) to immunotherapy were used to directly compare the numbers of VECs and T cells in those two cases (FIG. 6A). Interestingly, a correlation was observed between the number of VECs and T cell whether or not the patients were responding to immunotherapy, but samples from responsive patients had much higher number of T cells in the tumor and the correlation with the number of VECs was stronger. This seems to indicate that in unresponsive patients the VECs might be dysfunctional and unable to facilitate the infiltration of T cells in the tumor.

Similarly, a strong correlation was observed between VEC numbers and T cell numbers in human pancreatic tumors (FIG. 6F). Pancreatic tumors were also of particular interest because non-malignant pancreas samples as well as peritumoral duodenum samples were available. Although the non-malignant (NM) pancreas samples were not from the same patients as the pancreatic tumor samples, they were obtained from “healthy” areas of diseased pancreas, which allowed for these samples to be used as a healthier control to the pancreas tumor samples. Duodenum samples were obtained from patients undergoing a Whipple procedure where part of the duodenum is removed along with the pancreatic tumor so they can be considered peritumoral samples. VEC and T cell contents were measured in all samples and matched the number for each individual sample (FIG. 6H). Again, a strong correlation was observed between VEC and T cell numbers, and similarly to what was observed in the mouse MC38 model the peritumoral and the non-malignant tissues had much lower numbers of T cell and VEC suggesting that VEC are a key component of T cell infiltration in tumor.

Generally, the melanoma samples had higher T cell infiltration than the pancreas samples, in part because they came from patients who were responsive to immunotherapy. Thus, melanoma has been used as an example of immunogenic tumor in human while the pancreas tumor samples has been used as an example of non-immunogenic tumor in human.

To focus selectively on T cell recruitment, homing experiments with adoptively transferred tumor-specific effector and memory T cell subsets were performed (Weninger, W., M. A. Crowley, N. Manjunath, and U. H. von Andrian. Migratory properties of naive, effector, and memory CD8 (+) T cells. J. Exp. Med. 194, 953-966, (2001); Gerlach, C. et. al. The Chemokine Receptor CX3CR1 Defines Three Antigen-Experienced CD8 T Cell Subsets with Distinct Roles in Immune Surveillance and Homeostasis. Immunity, 45, 1270-1284, (2016)). To assess T cell interactions with tumor microvessels, RAG knockout (KO) mice were used in which fluorescent activated T cells were transferred after MC38 and B16 tumor had been allowed to grow in these mice. The absence of mature B and T cells in RAG KO mice means that all T cell infiltration in the tumor happened after the transfer of exogenous T cells and that the T cells could be tracked to compare their numbers in immunogenic versus non-immunogenic tumors. 24 hours after the transfer a sharp difference between MC38 and B16 T cell infiltration was observed (FIG. 6I). The number of VECs and T cells in B16 tissues were on par with peritumoral and healthy tissue numbers while MC38 had at least twice the same amount of both VECs and T cells. This confirmed that the presence of intratumoral T cells is mainly due to infiltration and not to intratumoral T cell proliferation.

Isolation and Characterization of Venular Endothelial Cells by scRNA-Seq

So far, the finding that an increase in T cell infiltration correlates with an increase in the number of VECs in the tumor has been established. This increase in intratumoral T cells seems to be due to an increase in T cell interactions with tumor microvessels. However, it is still unclear how these interactions take place and what makes VECs in immunogenic tumor more efficient at recruiting T cells. To address these questions, scRNA-Seq was used to compare the EC transcriptomes of the two subcutaneous murine tumors (MC38 and B16F10) described above and fresh patient-derived human melanoma and pancreatic cancer ECs. For each tumor, VECs and NVECs from tumoral and non-malignant tissues were analyzed. This strategy can be used to identify endothelial genes, including genes encoding cell surface and secreted molecules, that are uniquely upregulated in the tumor microvasculature and possibly specific to immunogenic tumor VECs.

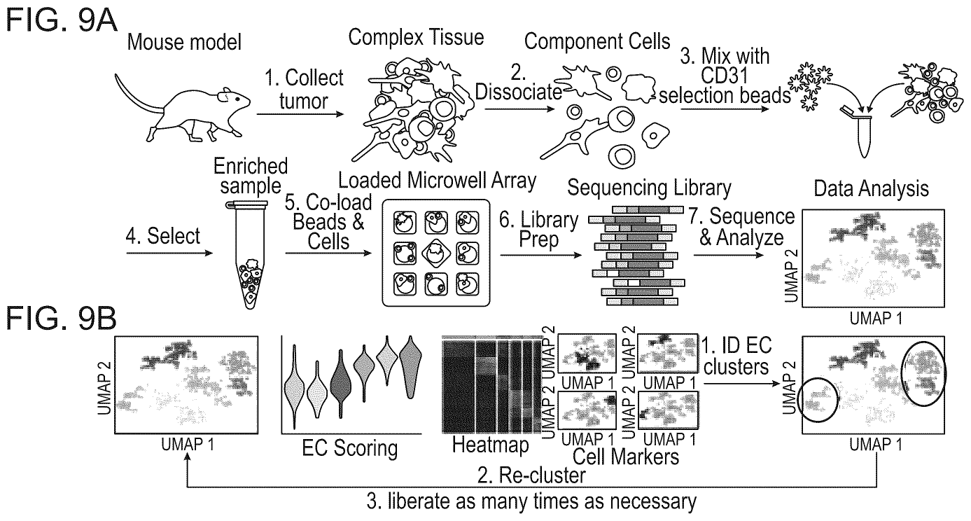

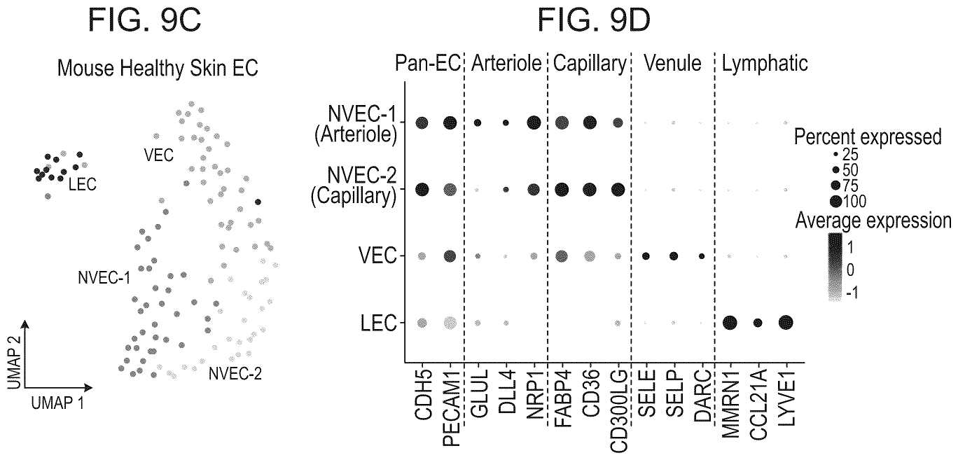

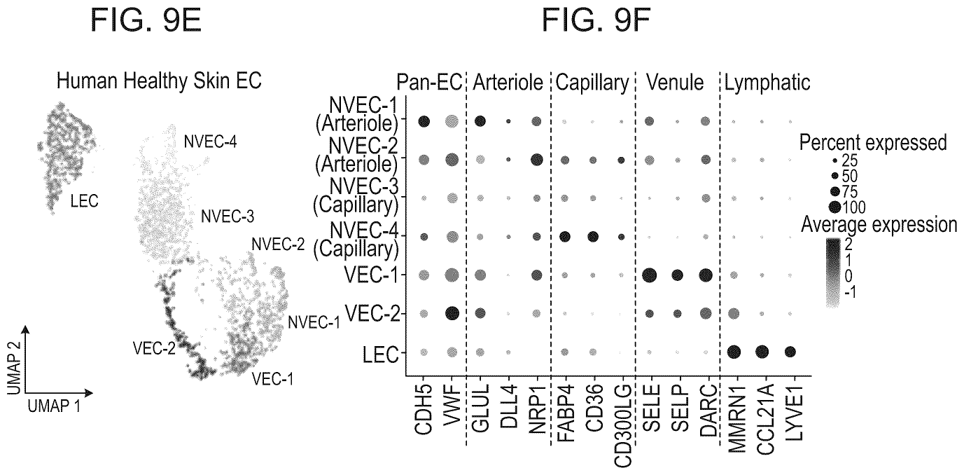

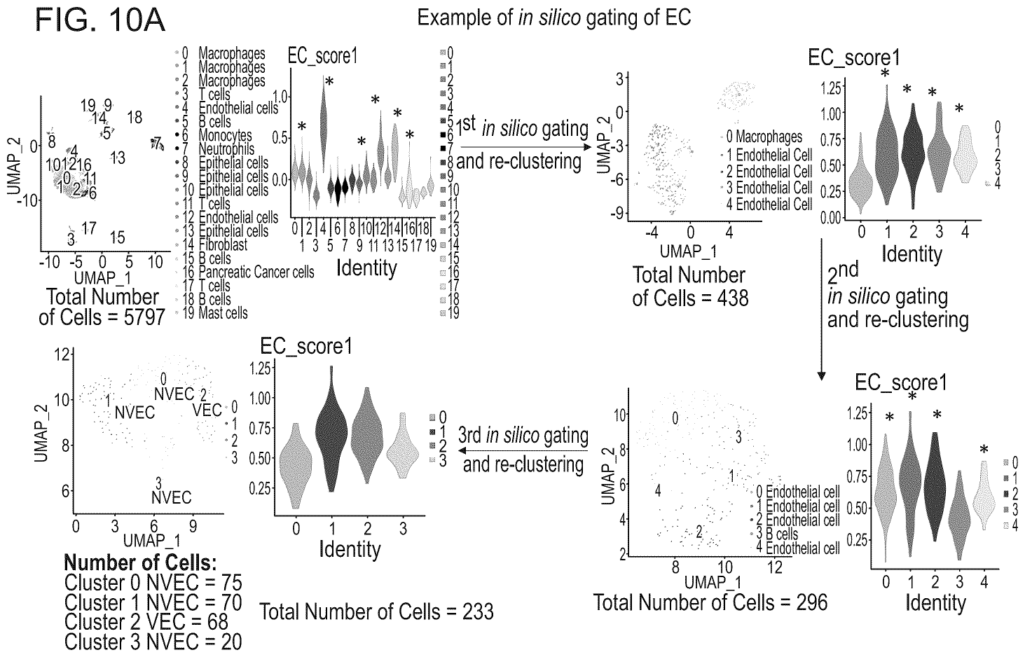

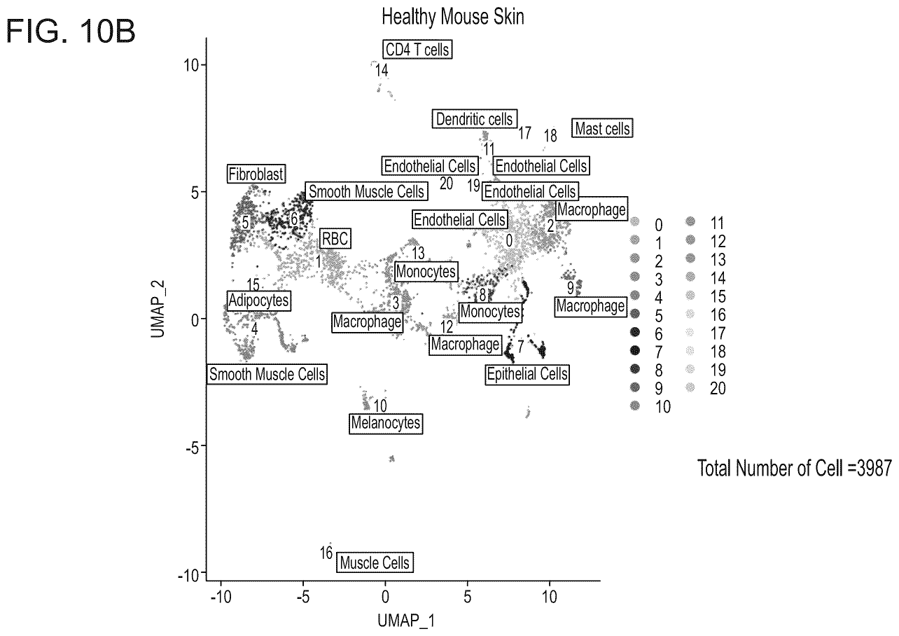

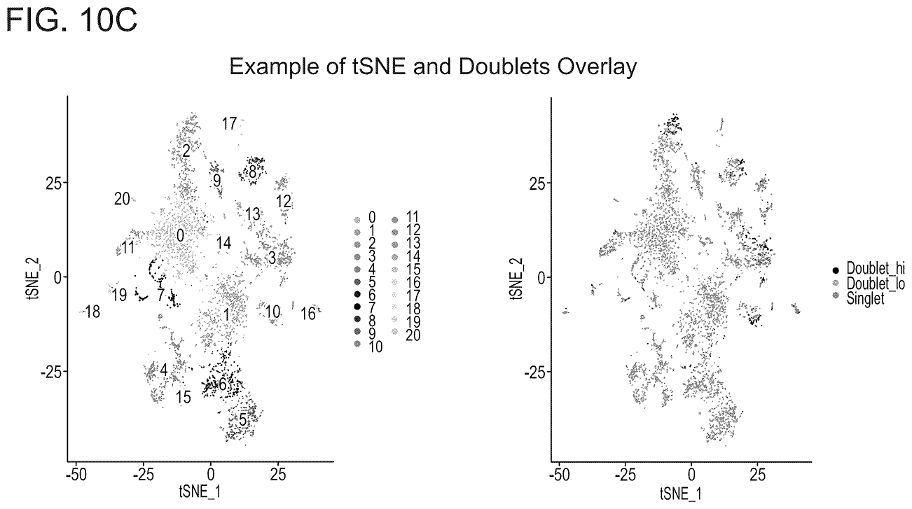

All mouse and human samples were processed using Seq-Well (FIG. 9A). Prior to Seq-Well, single-cell suspensions were prepared and were either used as is, or after CD45+ depletion or after CD31+ enrichment or a combination of both (see Table 1). Samples were then loaded on microwell arrays preloaded with barcoded beads. Libraries were prepared following the SeqWell protocol, and sequenced on an Illumina instrument. Segregation by enrichment method or by patient was never observed, so samples of the same tumor type were pooled after sequencing regardless of the preparation strategy for single cell suspension.

TABLE 1
Samples collected.

| Name | Sample type | Species | Enrichment |
|---|---|---|---|
| 180118 WT murine | dorsal skin | M | none |
| skin | | | |
| 180126 MC38 sort | Tumor MC38 | M | none |
| seq well | | | |
| | Healthy Skin | M | none |
| 180130 B16F10 Seq | Tumor B16F10 | M | none |
| Well | | | |
| | Healthy Skin | M | none |
| 180209 Seq Well | Tumor MC38 | M | None |
| | Tumor B16F10 | M | None |
| 180212 Seq Well | Tumor MC38 | M | None |
| | Tumor B16F10 | M | None |
| 180222 hSample | Pancreatic Tumor | H | none |
| PanT and Duo | | | |
| 180301 hSkin BP | Skin - array 1 | H | none |
| | Skin - array 2 | H | none |
| 180321 MC38 Seq | Tumor MC38 | M | CD31+ enrichment + spike with some −ve |
| Well | | | fraction |
| | Healthy | M | CD31+ enrichment + spike with some −ve |
| | | | fraction |
| 180322 hPT and Duo | Pancreatic Tumor | H | Depleted CD235a/b and CD45+ cells |
| 180327 B16F10 for | Tumor B16F10 | M | CD31+ enrichment + spike with some −ve |
| Sequencing | | | fraction |
| | Healthy | M | CD31+ enrichment + spike with some −ve |
| | | | fraction |
| 180328 B16F10 for | Tumor B16F10 | M | CD31+ enrichment + spike with some −ve |
| sequencing | | | fraction |
| 180403 MC38 Seq | Tumor MC38 | M | CD31+ enrichment + spike with some −ve |
| Well Rep2 | | | fraction |
| 180403 Healthy Pan | NM pancreas | H | CD31+ enrichment + spike with some −ve |
| | | | fraction |
| 180423 MC38 Seq | Tumor MC38 | M | CD31+ enrichment no other cells added |
| Well CD31 Pure#1 | | | |
| | Healthy | M | CD31+ enrichment no other cells added |
| 180425 B16 Seq Well | Tumor B16F10 | M | CD31+ enrichment no other cells added |
| CD31 Pure#1 | | | |
| | Healthy | M | CD31+ enrichment no other cells added |
| Healthy Panc | Healthy | H | CD31+ enrichment no other cells added |
| Human Pan Tumor | Pancreatic tumor | H | CD31+ enrichment no other cells added |
| and Healthy pancreas | | | |
| | Healthy Pancreas | H | CD31+ enrichment no other cells added |
| 180821 healthy human | Healthy Pancreas | H | CD31+ enrichment no other cells added |
| Pancreas | | | |
| 180910 Healthy | Healthy Pancreas | H | CD31+ enrichment no other cells added |
| Human Pancreas | | | |
| 181009 Human Pan | Healthy Pancreas | H | CD31+ enrichment no other cells added |
| Tumor and Healthy | | | |
| Pancreas | | | |
| | Pancreatic tumor | H | CD31+ enrichment no other cells added |
| 181009 Human Skin | Human Skin | H | CD31+ enrichment no other cells added |
| 181010 B16F10 and | B16F10 | M | CD31+ enrichment no other cells added |
| Peritumoral | | | |
| 181012 MC38 and | MC38 | M | CD31+ enrichment no other cells added |
| Peritumoral | | | |
| 181017 Human Skin | Human Skin | H | CD31+ enrichment no other cells added |
| 181018 Human Skin | Human Skin | H | CD31+ enrichment no other cells added |
| 181102 Human | Pancreatic Tumor | H | CD31+ enrichment no other cells added |
| Pancreas tumor | | | |
| 181105 Human | Melanoma | H | CD31+ enrichment no other cells added |
| Melanoma | | | |
| 181116 Human | Pancreatic Tumor | H | CD31+ enrichment no other cells added |
| Pancreatic Tumor and | | | |
| healthy pancreas | | | |
| 181116 Human | Healthy Pancreas | H | CD31+ enrichment no other cells added |
| Pancreatic Tumor and | | | |
| healthy pancreas | | | |
| 181120 Mouse | Healthy Mouse | M | CD31+ enrichment no other cells added |
| Healthy Skin | Skin | | |
| 181128 human Pan | Pancreatic Tumor | H | CD31+ enrichment no other cells added |
| Tumor and Pan | | | |
| Healthy | | | |
| 181128 human Pan | Healthy Pancreas | H | CD31+ enrichment no other cells added |
| Tumor and Pan | | | |
| Healthy | | | |
| 190130 hMelanoma | Melanoma | H | CD31+ enrichment no other cells added |
| 32719 MC38 | MC38 | M | CD31+ enrichment no other cells added |

M: mouse; H: Human; −ve fraction: CD45+ fraction collected after CD45 depletion.

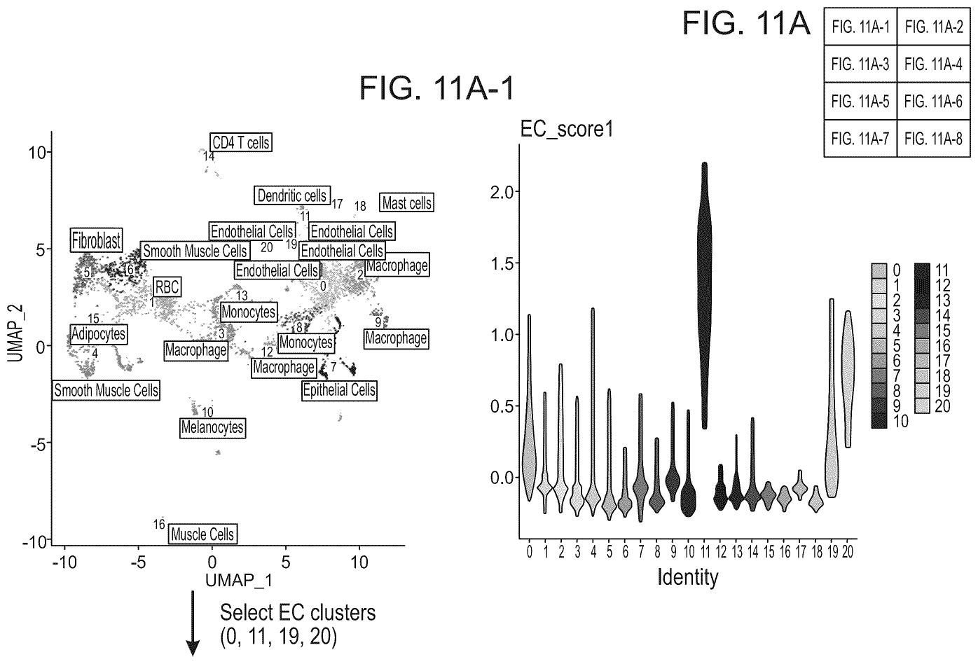

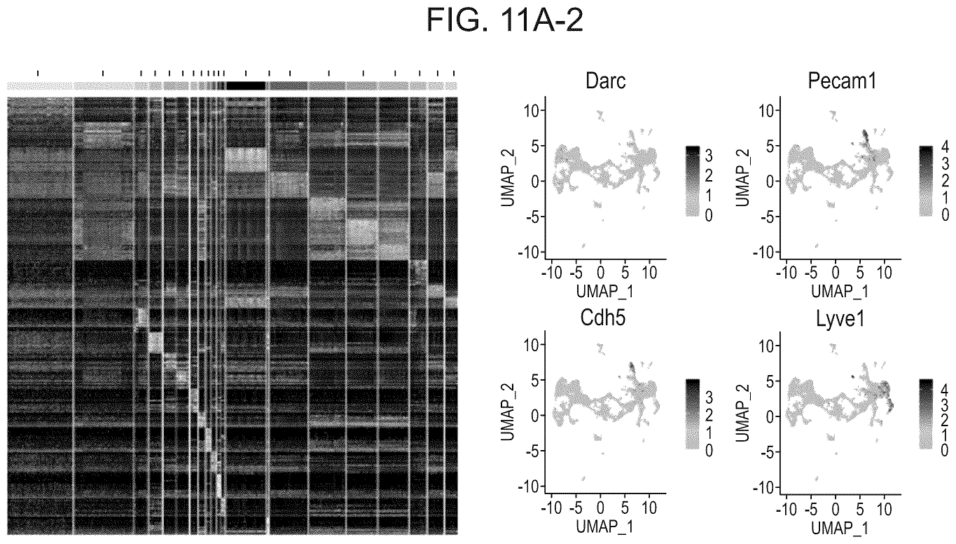

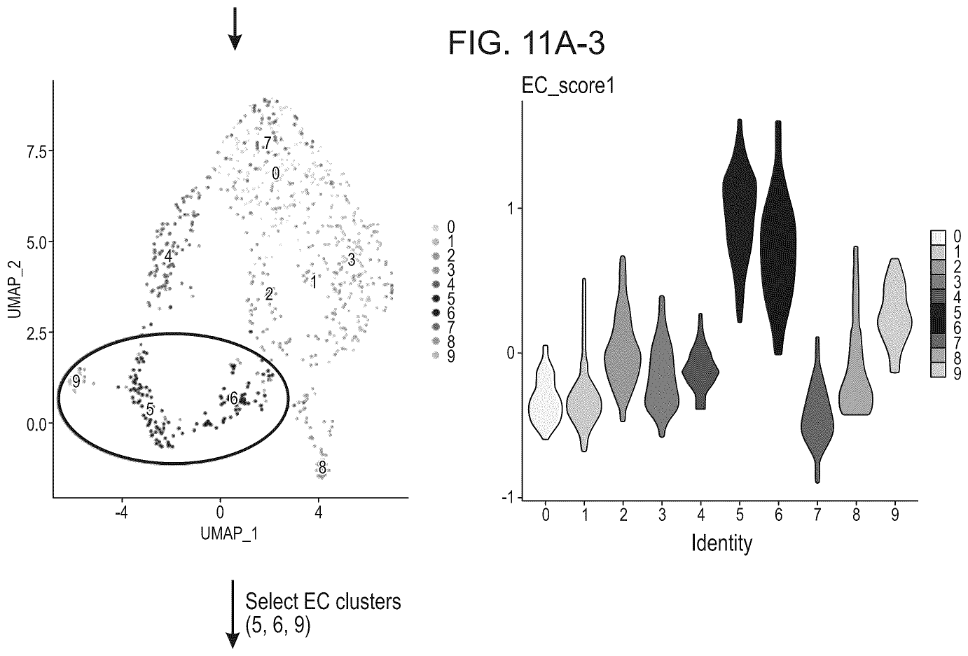

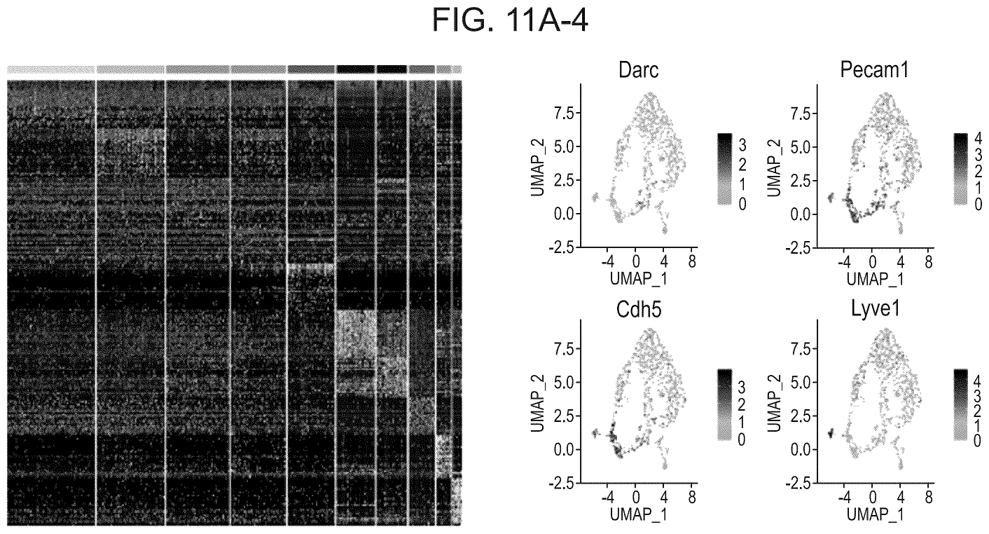

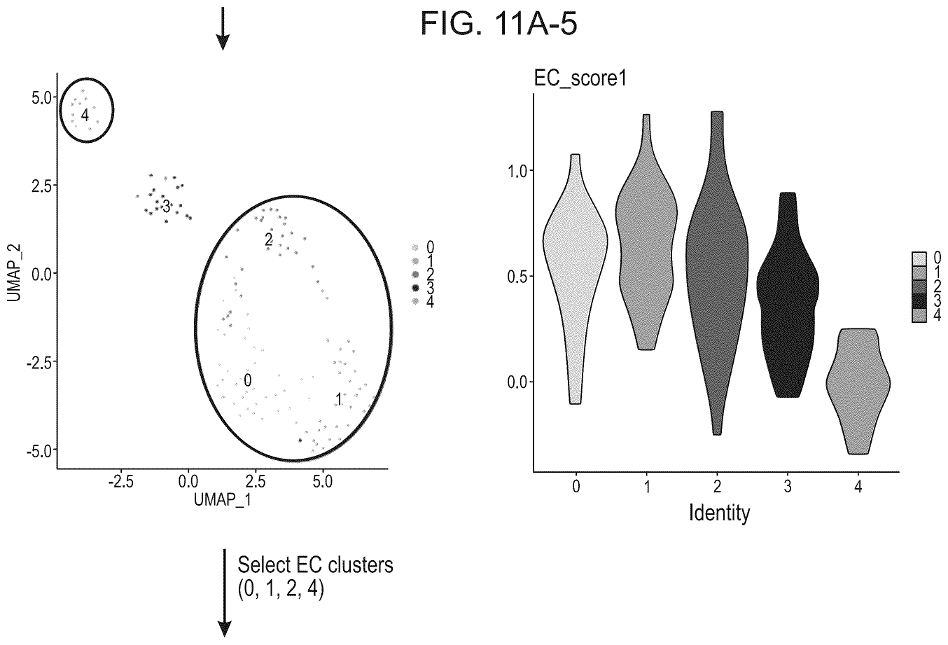

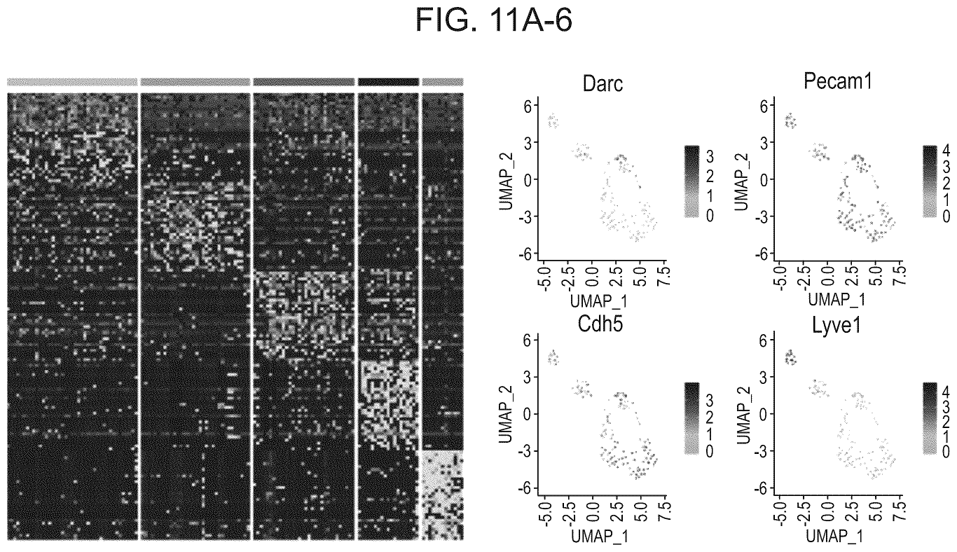

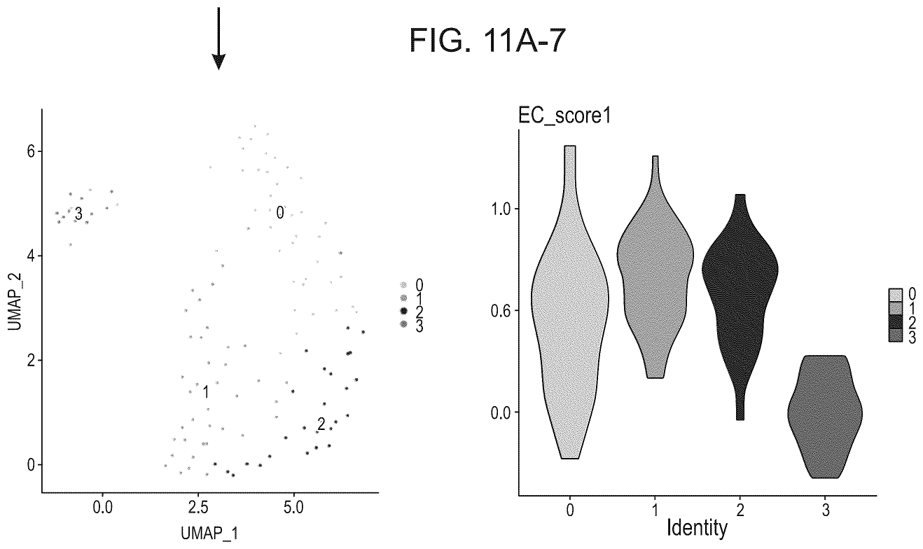

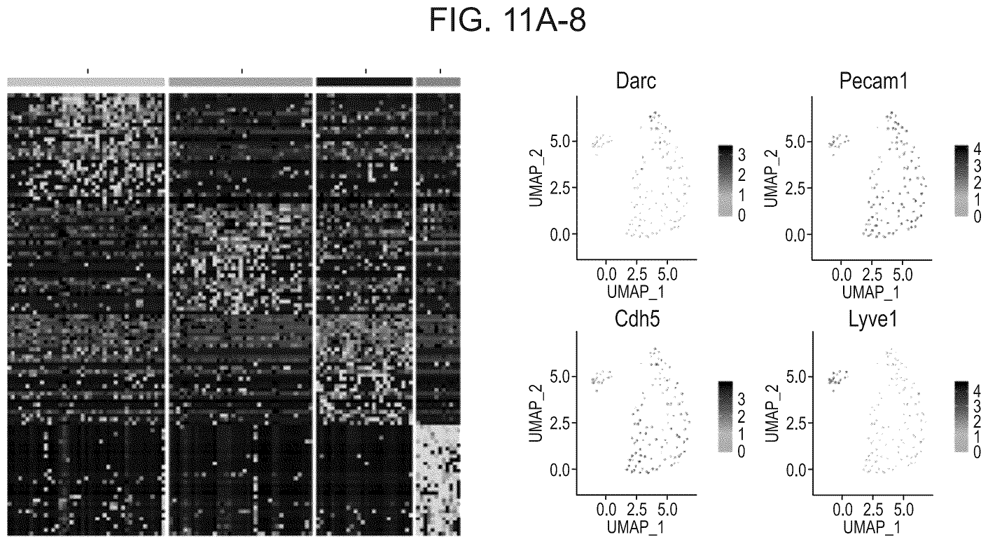

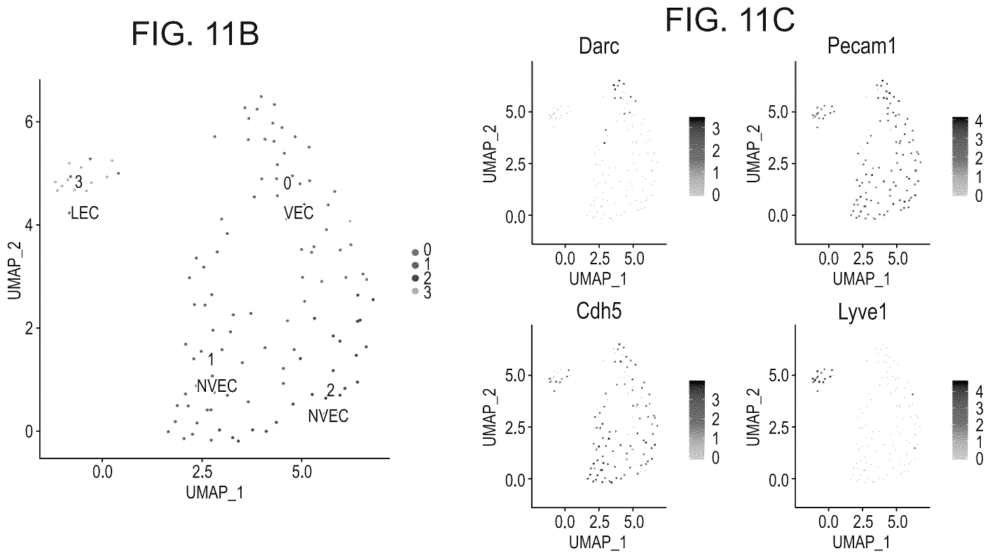

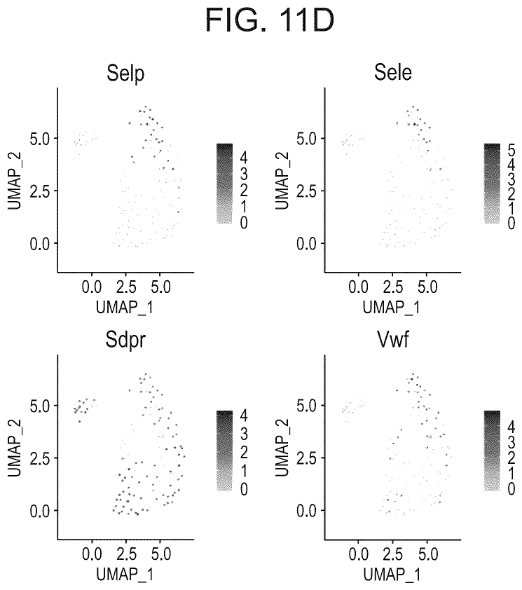

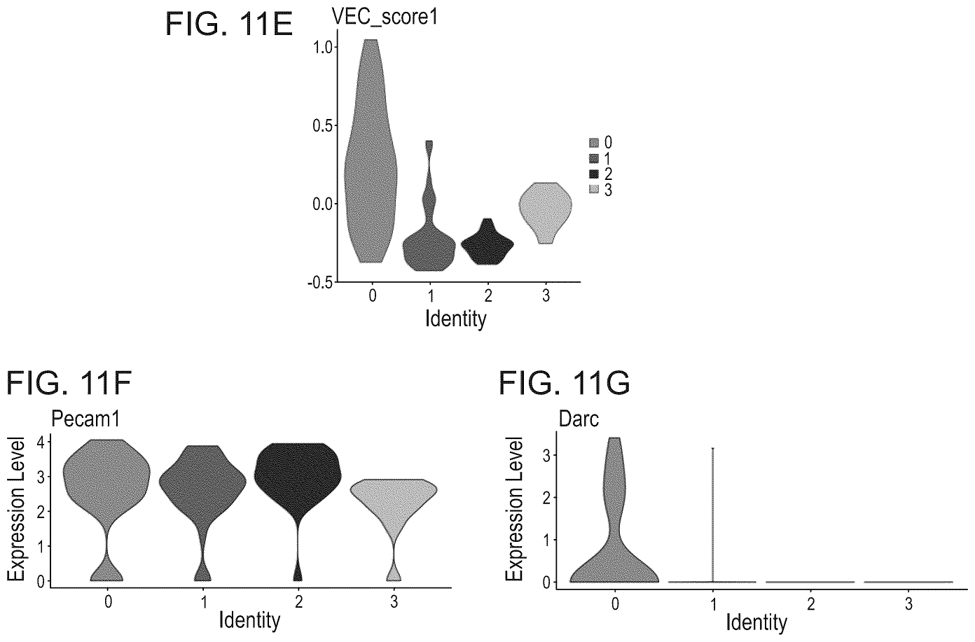

The first step was to develop an efficient methodology for isolating ECs. An iterative process that relied on several pieces of information to identify and isolate ECs was used (FIG. 9B). After clustering the cells using UMAP, the following was performed (1) differential expression to identify specific cell markers for each cluster to assess cell identity (2) heatmaps using those markers were used to help identify cluster(s) with similar gene expression patterns (3) EC scoring based on a list of previously validated markers (Table 2) to assess the EC-ness of a cluster. Based on those different criteria, clusters who were most likely to be containing ECs were isolated and re-clustered. The process was repeated until fully isolated ECs were acquired for each sample type—i.e. healthy skin. MC38 tumor, melanoma, etc. (FIG. 11A).

TABLE 2
Gene signatures used in EC and VEC module scoring.

| | |
|---|---|
| Mouse | Human |

| EC | VEC | EC | VEC |
|---|---|---|---|
| Tie1 | Selp | IFI27 | DARC |
| Mmrn2 | Sele | MGP | SELE |
| Eltd1 | Plvap | SDPR | PLVAP |
| Podxl | Vwf | RAMP2 | CLU |
| Flt1 | Icam1 | IGFBP6 | SELP |
| Selp | Lrg1 | SPARCL1 | ICAM1 |
| Pecam1 | Rasa4 | TIMP3 | LIFR |
| Gpr116 | Il6st | RGS5 | IL1R1 |
| Apold1 | Ctnnal1 | CAV1 | DUSP23 |
| Sele | Darc | VWF | LHX6 |
| Esam | Tmem252 | PTRF | UPP1 |
| Egfl7 | Upp1 | TM4SF1 | PDIA5 |
| Lyve1 | Nr2f2 | IGFBP7 | PRCP |
| Cyyr1 | Ehd4 | CLDN5 | VWF |
| Rasip1 | Syt15 | CPE | MCTP1 |
| Plvap | Sncg | SPARC | OLFM1 |
| Ptprb | Myc | IGFBP5 | LRG1 |
| Cdh5 | Tacr1 | DARC | IGFBP4 |
| Robo4 | Itgb4 | RAMP3 | MYRIP |
| Epas1 | Pdia5 | EMCN | VCAN |
| Vwf | Mctp1 | FCN3 | SYT15 |
| Gpihbp1 | Plekha7 | C7 | MYOF |
| Itga6 | Nuak1 | PTPRB | CSRP2 |
| Fgd5 | Zfp423 | ELTD1 | TACR1 |
| | Arrb1 | EGFL7 | FSTL1 |
| | Lepr | COL4A1 | IL13RA1 |
| | Pgm5 | ENG | CTNNAL1 |
| | Kank1 | AC011526.1 | NDRG1 |
| | Ptgs1 | CD34 | IL6ST |
| | Marveld1 | CDH5 | |
| | Spint2 | ADAMTS1 | |
| | Ret | TMEM100 | |
| | Lhx6 | LDB2 | |
| | Chp2 | LIFR | |
| | Igfbp4 | FBLN2 | |
| | Procr | SELE | |
| | Setbp1 | JAM2 | |
| | Bace2 | CYYR1 | |
| | Hoxd10 | IL33 | |
| | Tll1 | CTNNAL1 | |
| | Golm1 | PRSS23 | |
| | Olfml2a | PODXL | |
| | Vim | EPAS1 | |
| | Pam | PLVAP | |
| | Asap3 | PLCB4 | |
| | Slco2a1 | ENPP2 | |
| | Nmt2 | CRIP2 | |
| | Ldlrap1 | CLU | |
| | | GNG11 | |
| | | CALD1 | |
| | | ESAM | |
| | | APOLD1 | |
| | | ARHGAP29 | |
| | | FLT1 | |
| | | SOCS3 | |
| | | CNN3 | |
| | | CD36 | |
| | | CFH | |
| | | RNASE1 | |
| | | IGFBP3 | |
| | | CRIM1 | |
| | | MT1X | |
| | | RGS16 | |
| | | RND1 | |
| | | ADIRF | |
| | | CXCL2 | |
| | | YBX3 | |
| | | EMP1 | |
| | | SERPING1 | |
| | | CALCRL | |
| | | TNFSF10 | |
| | | C8orf4 | |
| | | EGR1 | |
| | | IL6 | |
| | | SPTBN1 | |
| | | IGFBP4 | |
| | | H19 | |
| | | MTUS1 | |
| | | IL1R1 | |
| | | FABP4 | |
| | | IFITM3 | |
| | | MKL2 | |
| | | CD320 | |
| | | CLIC4 | |
| | | TSC22D1 | |
| | | MAFF | |
| | | ICAM1 | |
| | | RDX | |
| | | ATF3 | |
| | | APOD | |
| | | CCL2 | |
| | | CXCL12 | |
| | | IL6ST | |
| | | GSN | |
| | | TIMP1 | |
| | | ZFP36 | |
| | | ITM2B | |
| | | NCOA7 | |
| | | GADD45B | |
| | | MT2A | |

Second, healthy skin controls for mouse and human were analyzed to ensure that known EC biology was observed and recapitulated. In the mouse healthy skin EC, 4 clusters were observed (FIG. 9C). When looking at the genes expressed by these clusters, the expression of pan-EC genes (CDH5 and PECAM1) in all cells was observed, confirming that ECs were successfully isolated. Genes known to be selectively expressed by EC subsets¹⁶⁻¹⁹ were used to further identify the clusters. One cluster of LECs, one cluster of VECs, one cluster of capillary ECs and finally one cluster of arteriole ECs were observed. Capillaries and arterioles presented more as a spectrum while VECs and LECs were clearly defined subsets (FIG. 9D). Similarly, out of the seven clusters observed in the human healthy skin 2 clear clusters of VEC, one cluster of LEC and 4 clusters of NVEC were observed. The 4 clusters of NVEC seemed to present a spectrum going from arterioles in NVEC-1 to capillaries in NVEC-4 (FIGS. 9E and 9F).

Immunogenic Tumor VEC More Closely Resemble Non-Tumor VEC and their Profile is Conducive to Recruit Immune Cells

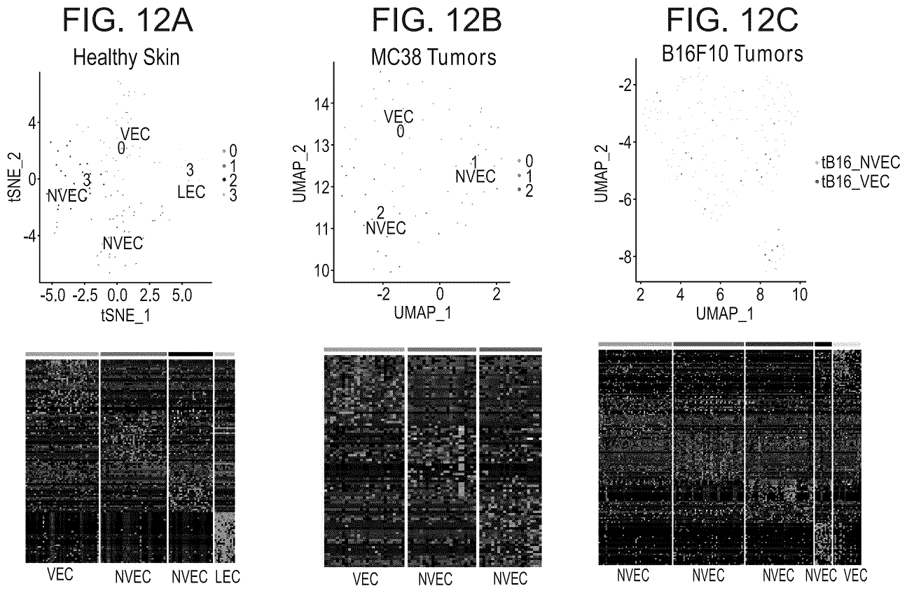

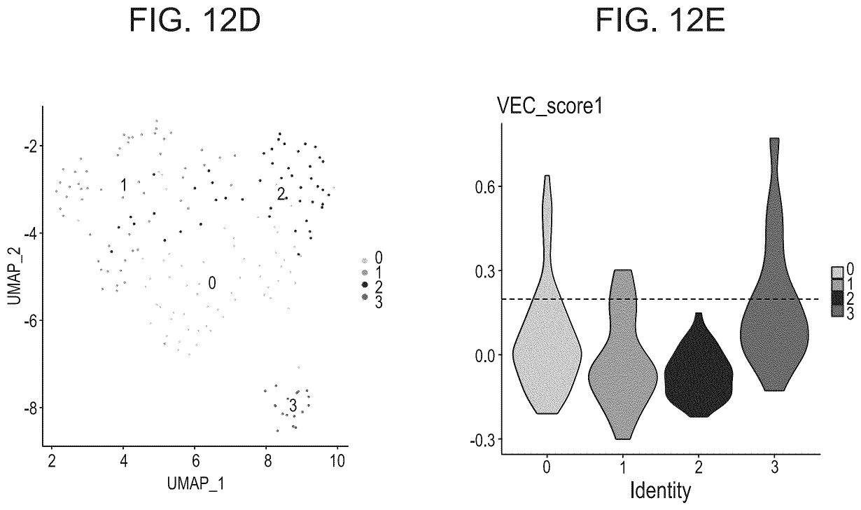

Next, upon establishing that endothelial cells were successfully isolated and analyzed, NVEC and VEC signatures in the immunogenic and non-immunogenic mouse models (MC38 and B16 respectively) were analyzed. To do so MC38 and B16 samples were processed according to the protocol described in FIGS. 9A and 9B. After final clustering, an individual VEC cluster in MC38 was identified (Similar to what was observed in the healthy skin, FIGS. 12A-12C). However, for B16 the VECs were not distinct enough from NVECs to separate into their own cluster. As a result, module scoring based on a gene list curated from genes upregulated in healthy skin VEC was used to identify VECs (Table S2). Using this strategy, VECs that were spread between all the different clusters were identified (FIGS. 12A-12C).

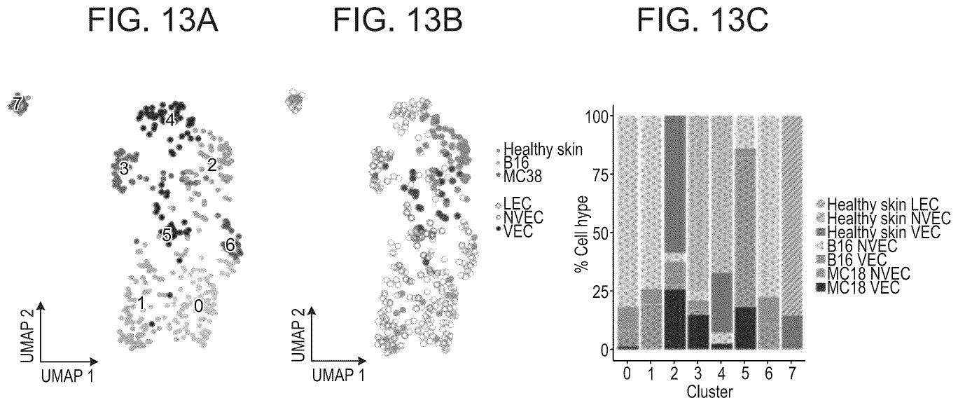

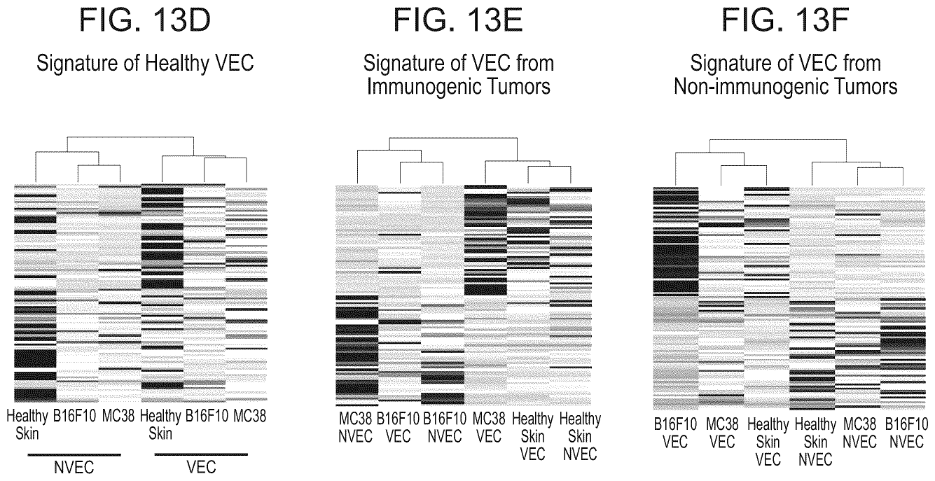

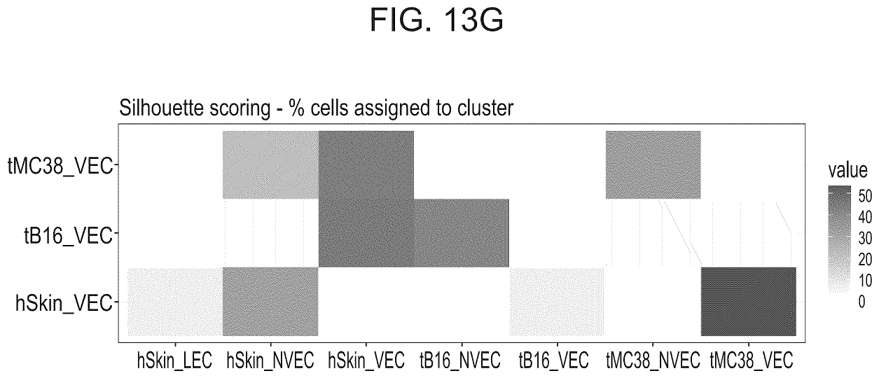

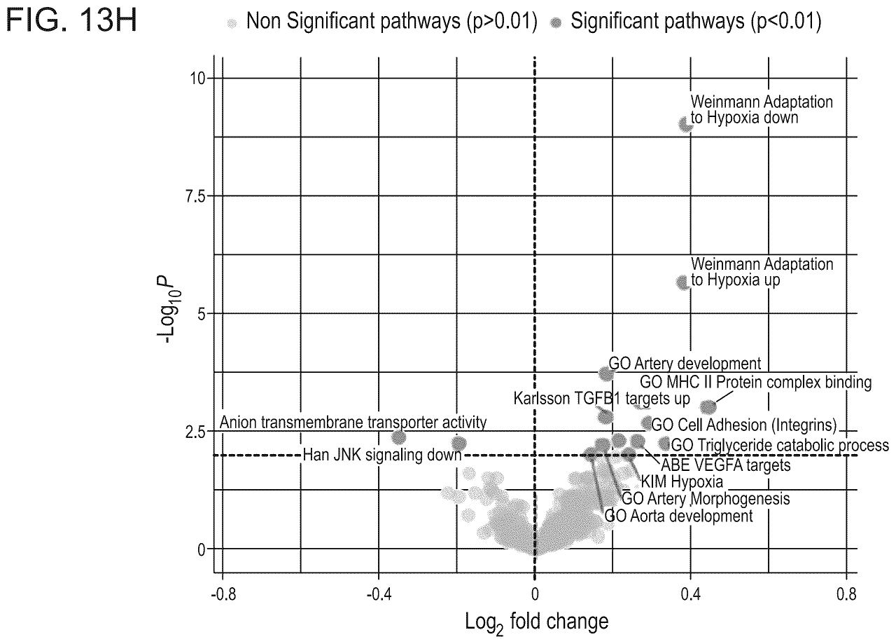

To determine the similarities between healthy, MC38 and B16 VECs and NVECs, all ECs from these samples were pooled together and an unsupervised UMAP clustering was run (FIGS. 13A and 13B). Seven distinct clusters were observed, and cells from different origins were compared to determine how they were spread between clusters. VECs from healthy skin and MC38 seemed to cluster in close proximity while VECs from B16 were spread all over the map. Checking for the exact division between clusters (FIG. 13C), cluster 2 contained mostly VECs from the healthy and MC38 samples while the VECs from B16 seemed to be spread fairly equally between clusters 0-3 and 5 and 6. This suggests that MC38 VECs are more similar to healthy VECs than B16 VECs.

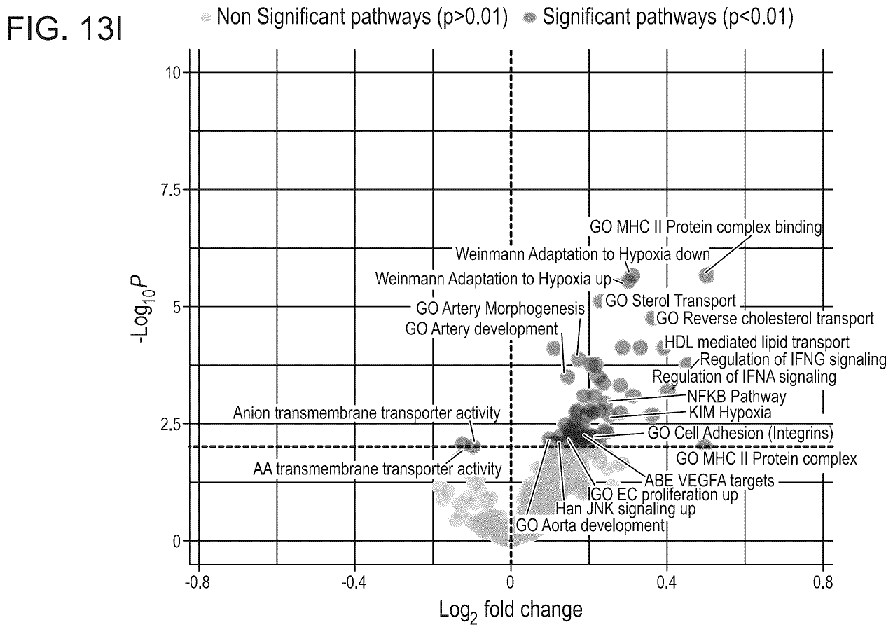

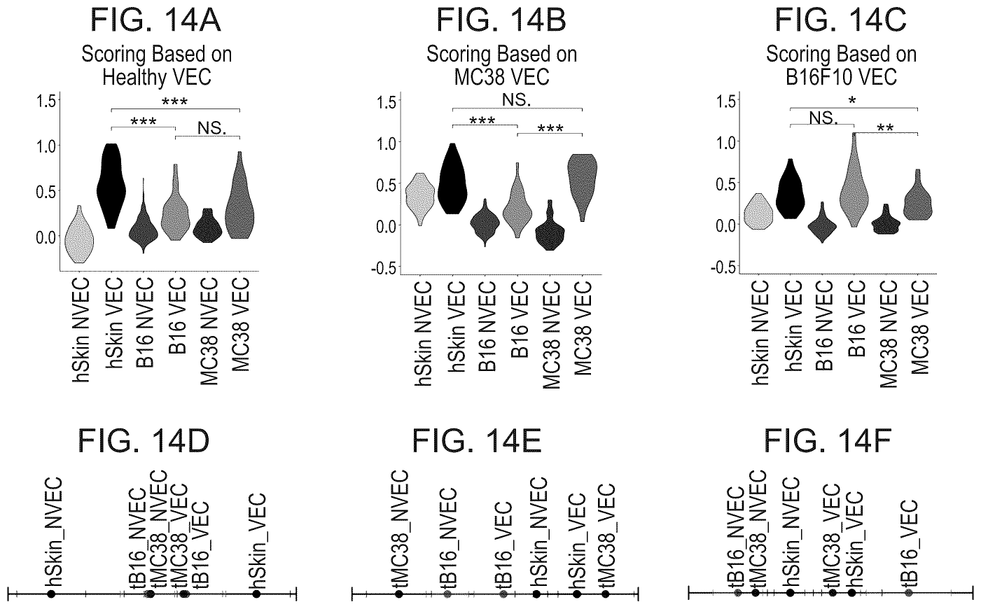

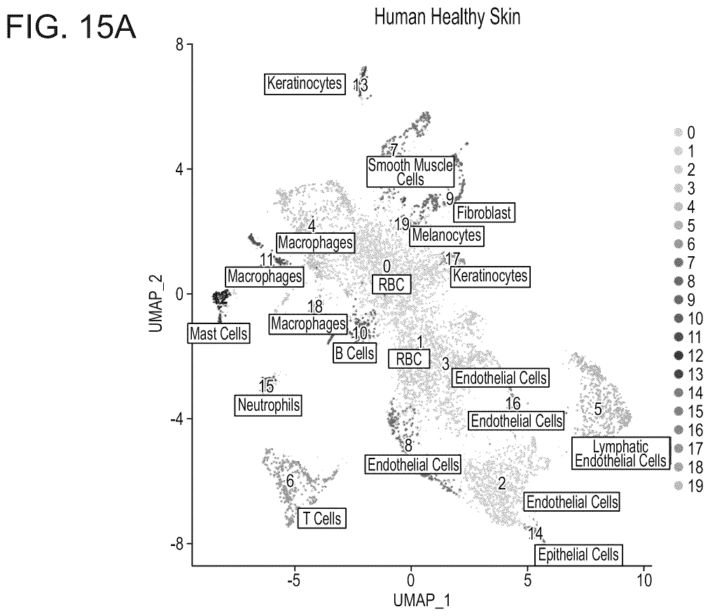

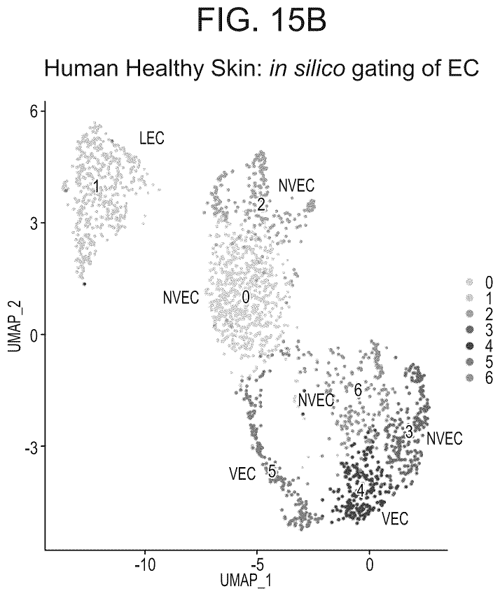

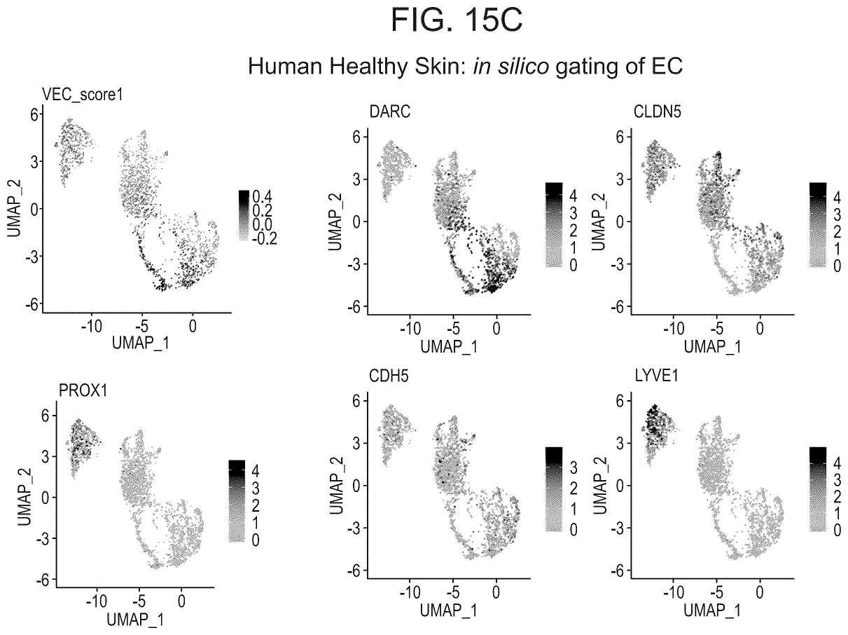

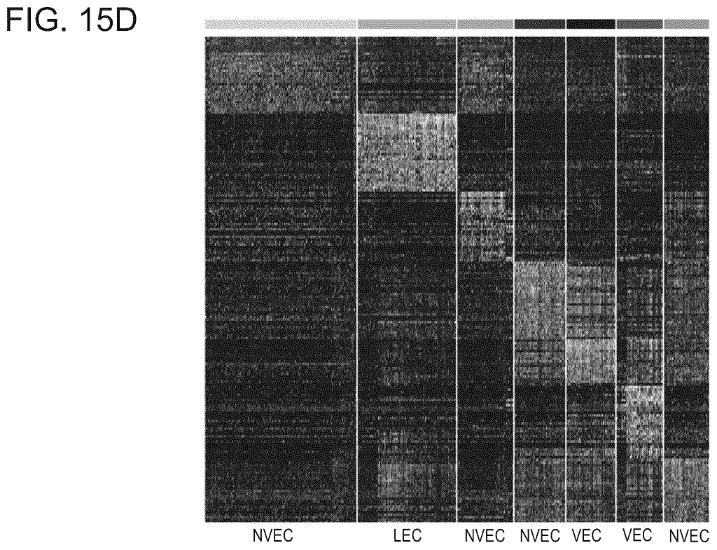

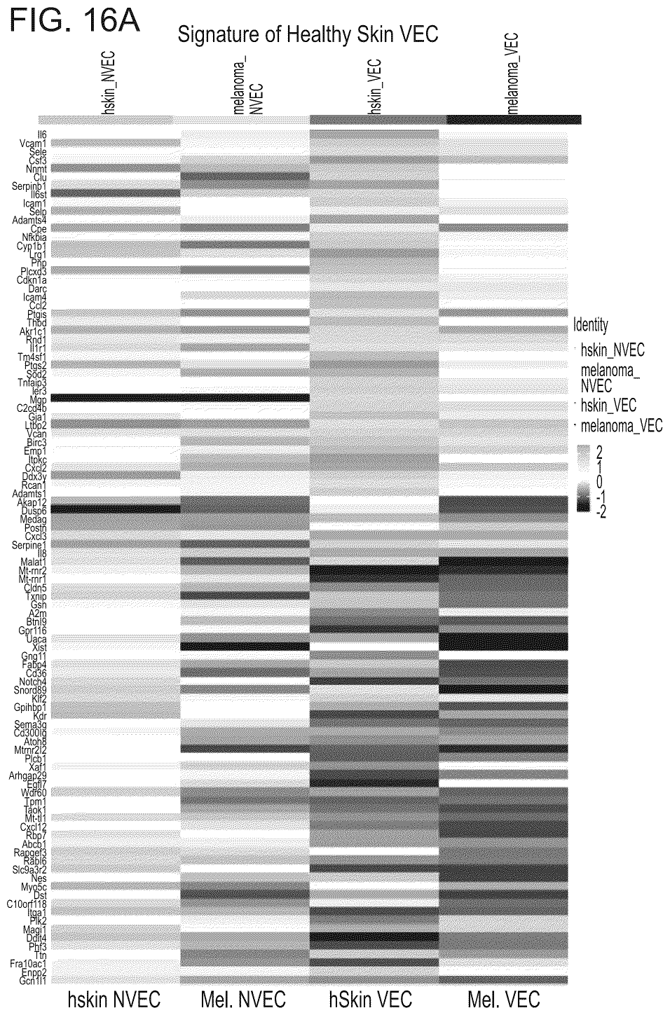

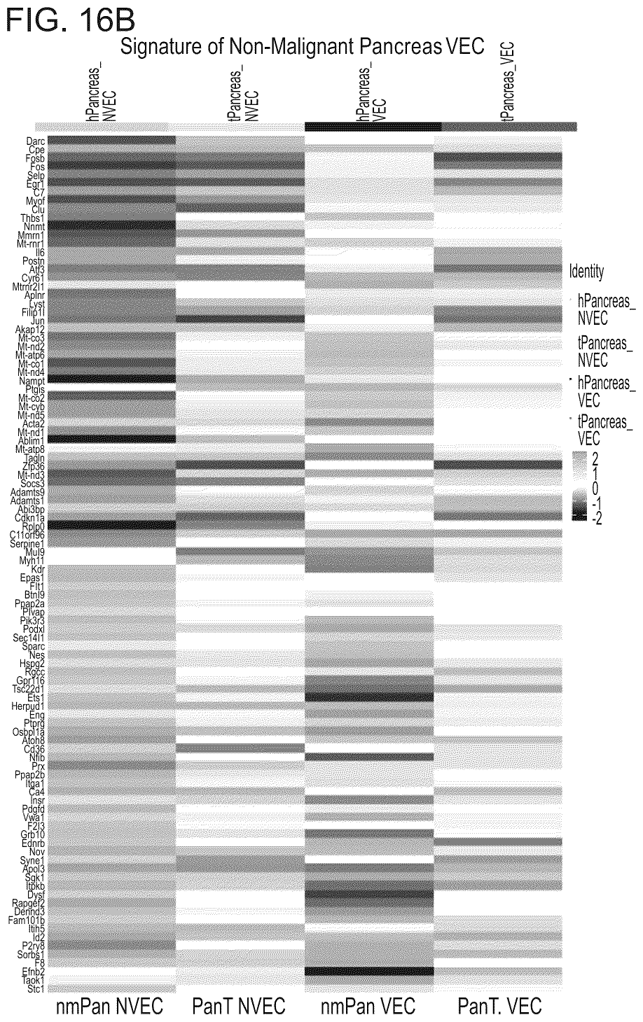

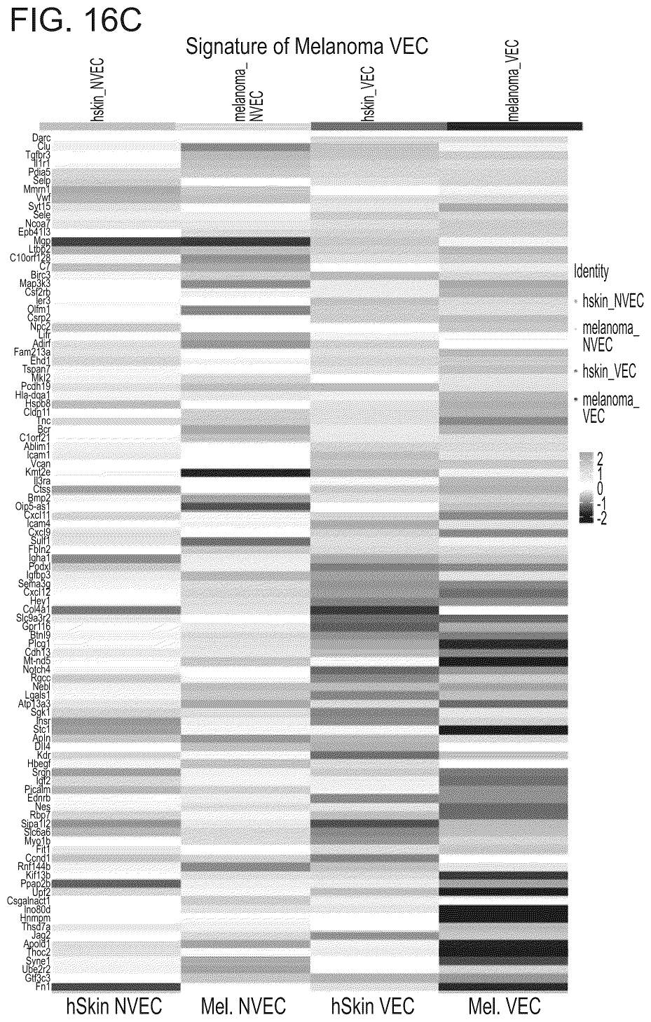

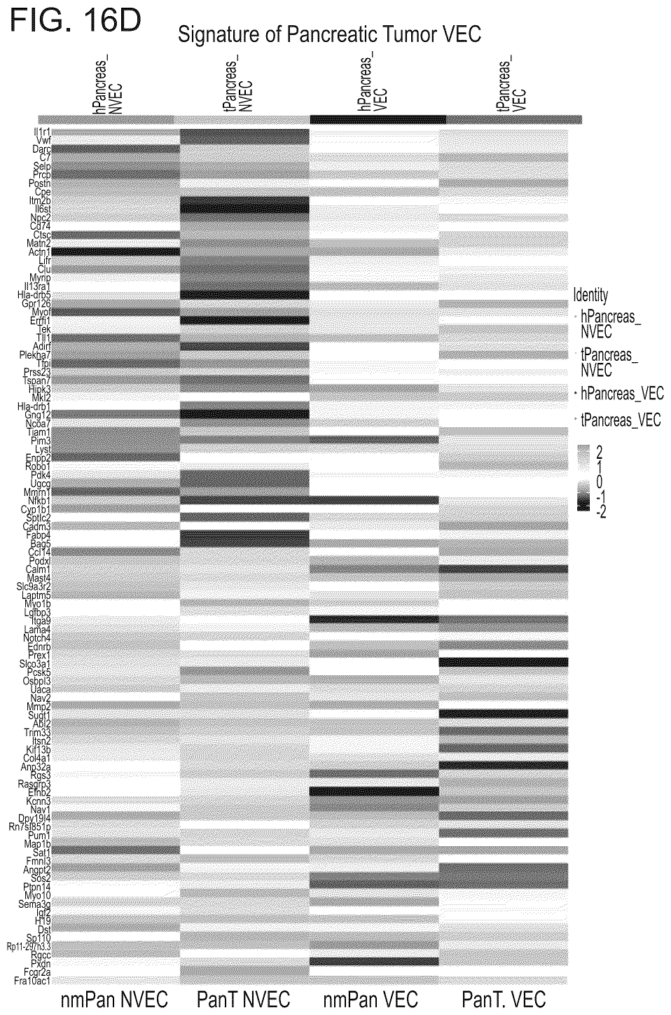

Silhouette algorithm²⁰ was used to assess the similarities between different subsets. Briefly, the silhouette algorithm evaluated which group of cells a cell is more closely related to, i.e. if the cell had not been assigned to its original group of cells silhouette will determine in which group it would have been placed. Silhouette was used on VECs from healthy skin, MC38 and B16 to see where those cells would fall (FIG. 13G). Most of the healthy skin VECs ended up being placed with the MC38 VECs showing that the healthy VECs resemble MC38 VECs more closely than B16 VECs. MC38 VECs and B16 VECs were split pretty equally between healthy VECs and their matched NVECs. This suggested the presence of a core VEC signature that is maintained in health and disease states. Other strategies based on module scoring were used to assess similarities between the different subsets providing similar results (FIGS. 14A-14F).

Thus, gene signatures from each VEC subset were analyzed and compared to each other. The differentially expressed genes between VECs and NVECs in each sample were looked at and the top and bottom 50 genes were picked. Those were plotted as a heatmaps against each other. Looking at the healthy signature, most of the upregulated genes are typical VEC genes (SELP, SELE, DARC, IL6ST, VWF) that appear to also be upregulated in MC38 and B16 VECs. A few other upregulated genes that are common between samples are new genes that had not been identified as VEC specific previously (CADM3, LRG1) (FIG. 13D). CADM3 (Cell Adhesion Molecule 3) is part of the Ca2+-independent immunoglobulin (Ig) superfamily that participate in the organization of epithelial and endothelial junctions,²¹ and LRG1 (Leucine Rich Alpha-2-Glycoprotein 1) has been shown to be involved in promoting neovascularization through causing a switch in transforming growth factor beta (TGFβ) signaling in endothelial cells.²² Both of those genes present new interesting targets for selective identification and isolation of VEC and would require to be further validated. Overall the genes differentially expressed in healthy skin VECs mostly revealed a core VEC signature that can be used to identify and select VECs regardless of health or disease.

The MC38 and B16 VEC signature on the other hand revealed more specific programs at play in the immunogenic and non-immunogenic context. In MC38, upregulation of genes involved in DNA damage protection (TMEM109), cell adhesion and migration (LAMB2), and proliferation and angiogenesis (TGFB, FOS) was observed (FIG. 13E). Meanwhile, in B16, upregulation of anti-inflammatory genes (NFKBIA, NFKBIZ, NKRF), as well as regulator of cell growth and proliferation (NDRG1, FOSB) was observed (FIG. 13F). This suggests that while VEC will grow and participate in angiogenesis in both MC38 and B16, they will serve different purpose in each tumor. In MC38 they seem to contribute to T cell recruitment through the expression of cell adhesion proteins, while the expression of anti-inflammatory proteins in B16 will drive down T cell infiltration.

Those preliminary observations were confirmed upon performing gene set variation analysis (GSVA)²³ using a previously curated list of 615 vascular related gene sets that were selected from the Molecular Signatures Database (MSigDB).²⁴ As expected, a set of common pathways between MC38 and B16 was observed. Those are mostly related to hypoxia, angiogenesis and EC proliferation. All of these are expected in a solid tumor environment as hypoxia is one of the main features of solid tumors and is known to promote angiogenesis and thus EC proliferation.²⁵ Pathways that are specific to MC38 or B16 were also observed. In MC38, upregulation was observed of inflammation, with several interferon and virus response pathways, as well as an increase in sterol and lipid transport and production, which are known for their protective effects on EC (antioxidant, anti-protease, anti-thrombotic, anti-apoptotic, etc.).²⁶ Active sterol and lipid transport are also a hallmark of healthy EC operation, highlighting once again the similarities between MC38 VECs and healthy VECs. In B16, upregulation was observed of TGFβ which is involved in cell proliferation and a downregulation of the JNK pathway which has been linked to a reduction in inflammation and T cell recruitment in ECs.²⁷

Human and Mouse Immunogenic VECs have a Common Transcriptional Profile

Human samples (healthy skin, melanoma, NM pancreas, pancreatic tumor) were used to see if a pattern similar to what was observed in mouse immunogenic and non-immunogenic tumors emerged. The comparison between healthy skin and melanoma samples was used to look for hallmarks of immunogenic tumors and the comparison between NM pancreas and pancreatic tumor was used to look for hallmarks of non-immunogenic tumors.

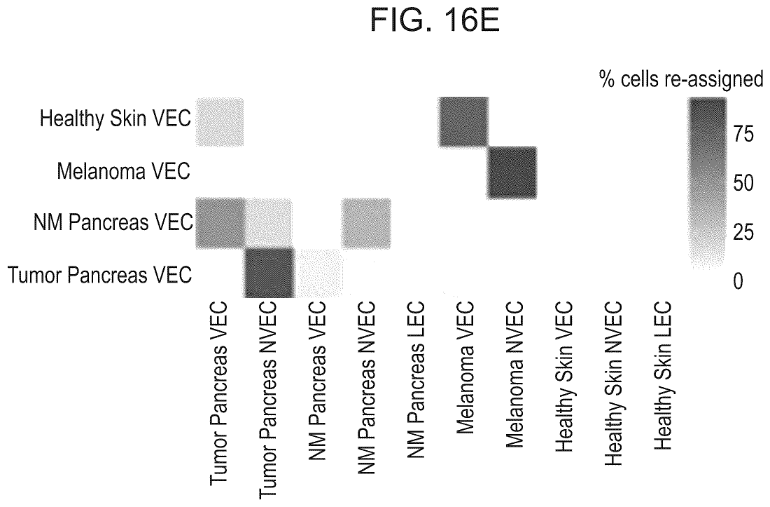

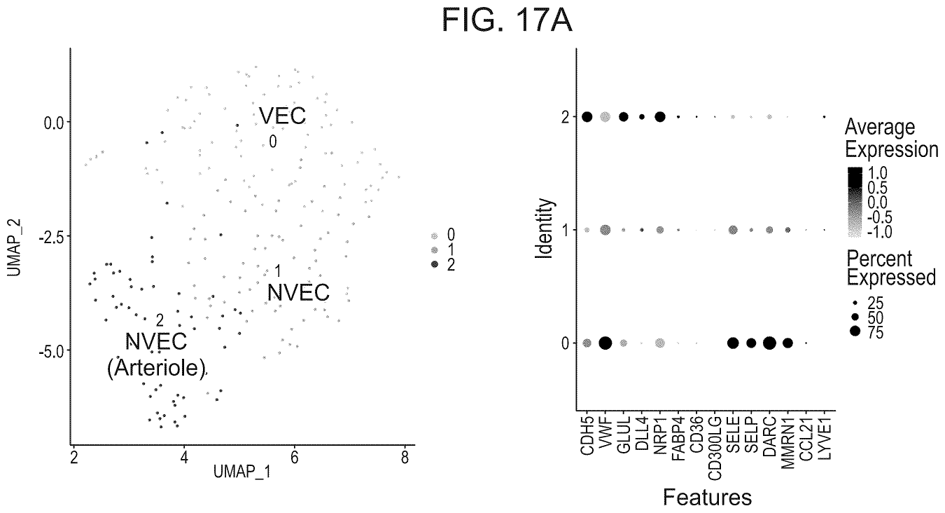

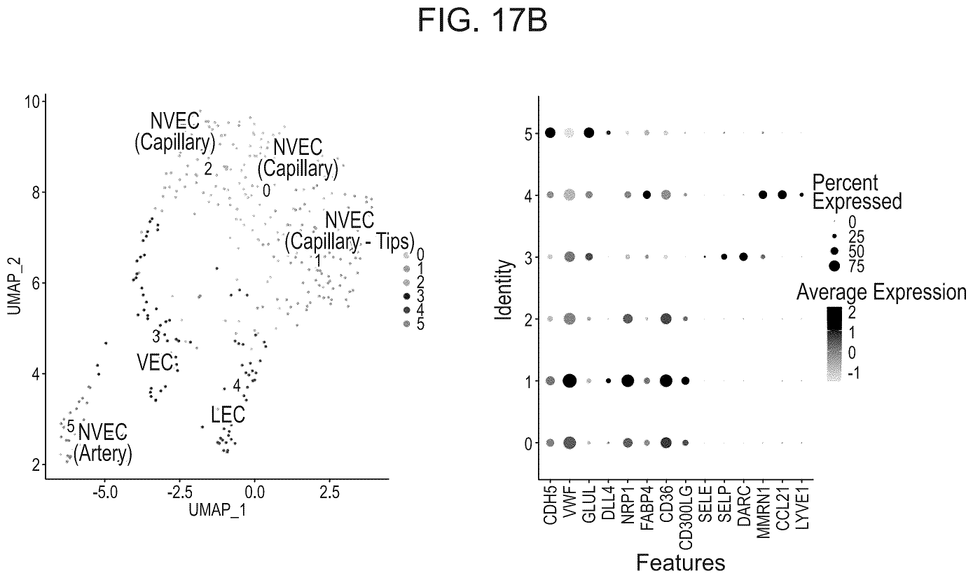

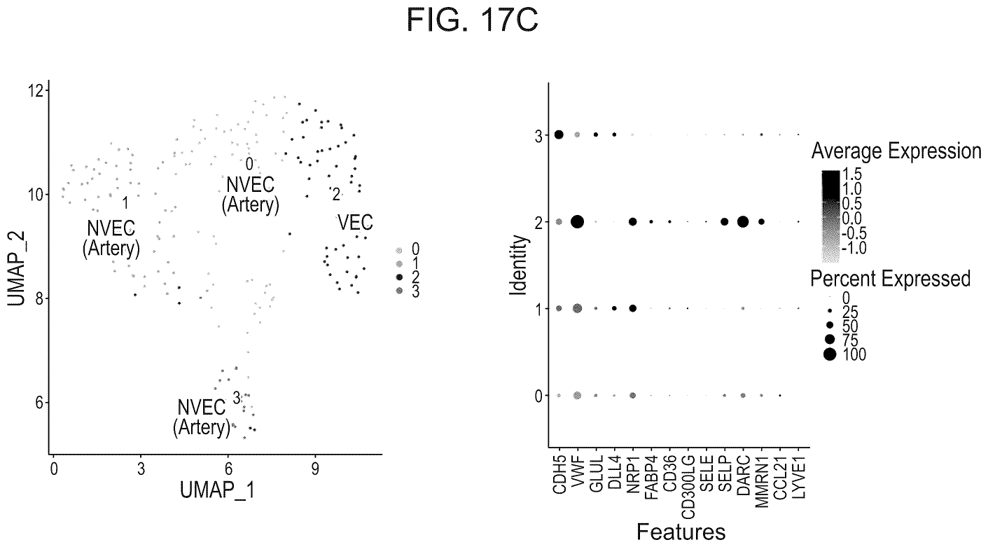

ECs were isolated from each sample, and processed by unsupervised clustering and cluster identification. In all cases, a clear separation between VEC and NVEC was observed (FIGS. 17A-17C). Human skin and melanoma ECs were pooled together and the cells clustered. The same process was repeated for NM pancreas and pancreatic tumor ECs. The four samples were not pooled together as they're coming from very different tissues and would then separate according to tissue type rather than EC subsets.

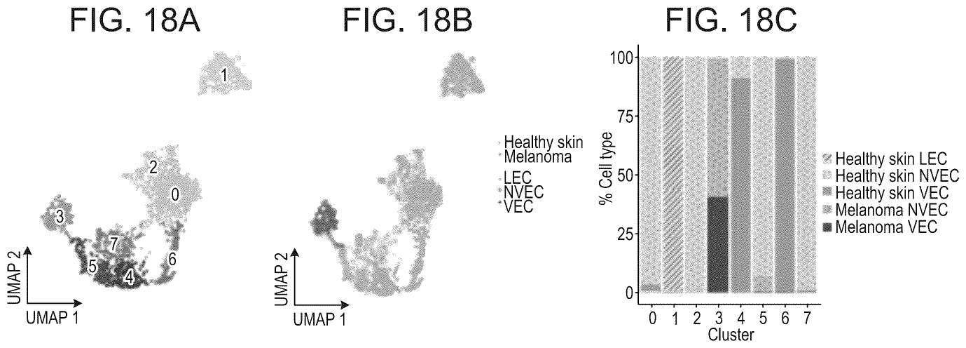

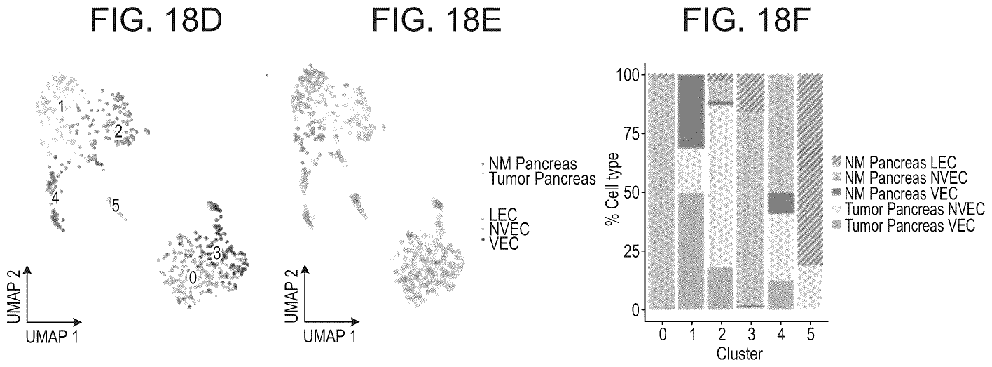

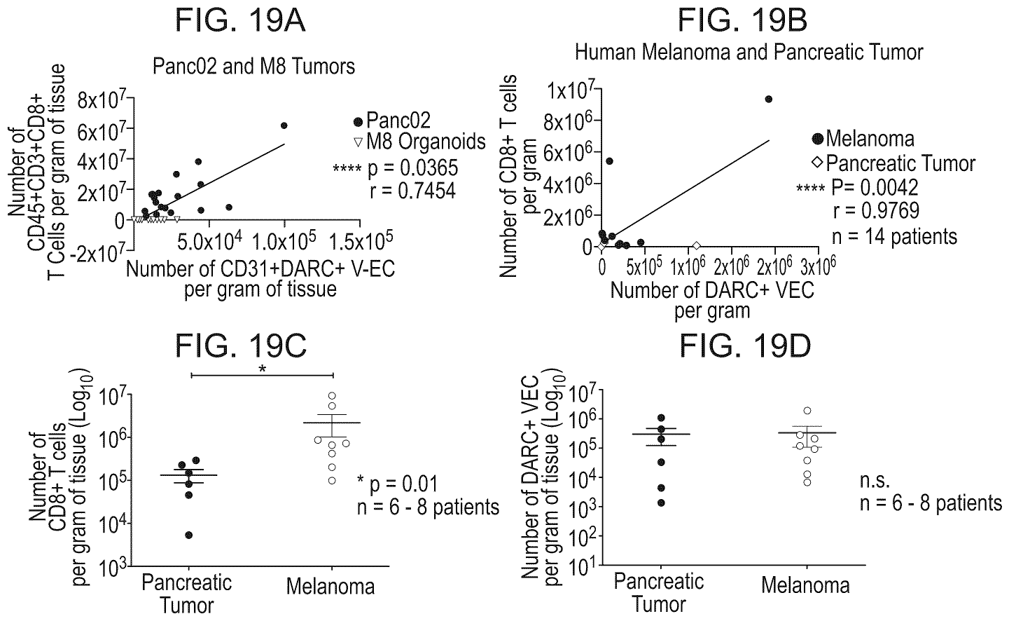

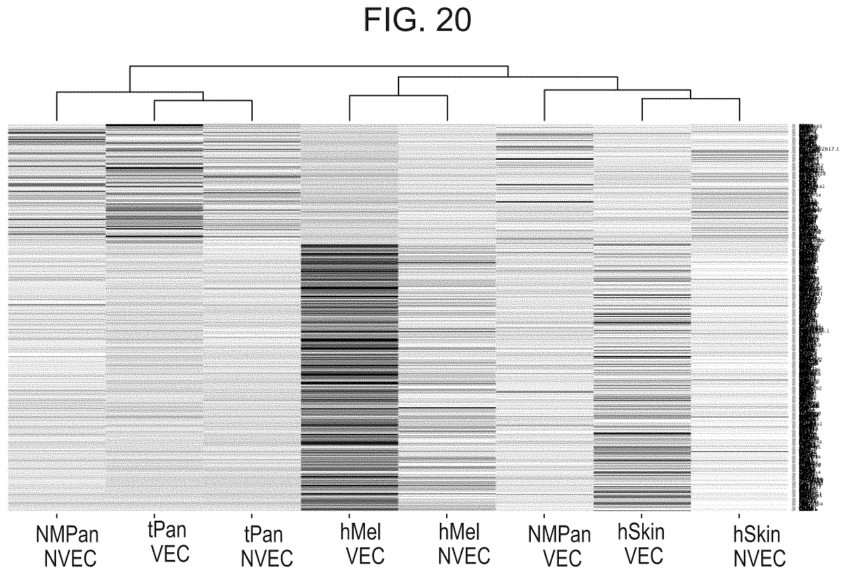

When pooling melanoma and healthy skin, a segregation by sample type was observed (FIGS. 18A-18C). The genes driving that separation are mostly heat shock protein and immune response genes that are both known to be upregulated in melanoma compared to healthy skin.²⁸⁻³⁰ The pooling of pancreas samples did not lead to a stark segregation between non-malignant and malignant samples, clustering based mostly on cell type was observed (FIGS. 18D-18F). Unlike the healthy skin control the non-malignant pancreas control was taken from a disease-free area of the pancreas of a non-healthy subject which partially explains the increased similarities between NM pancreas and pancreatic tumor when compared to healthy skin and melanoma.

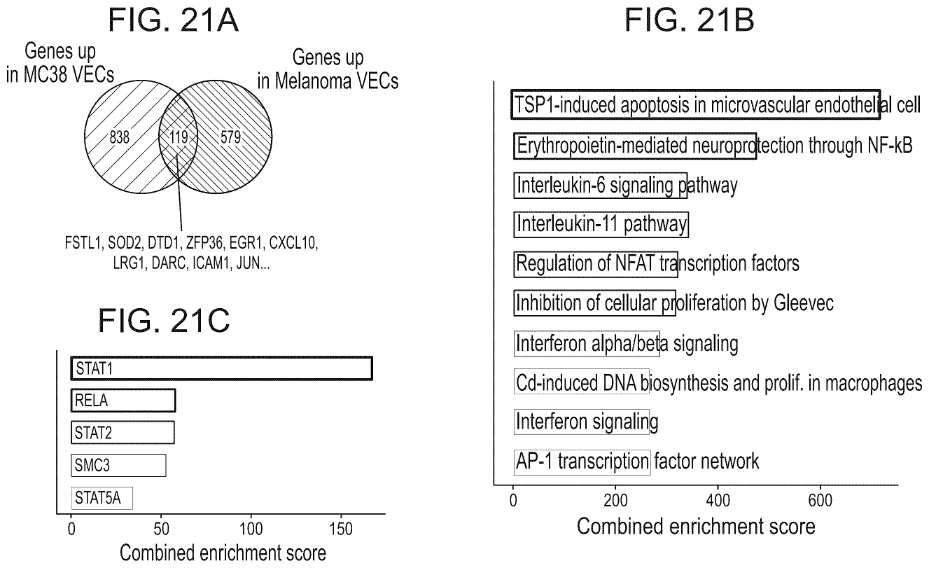

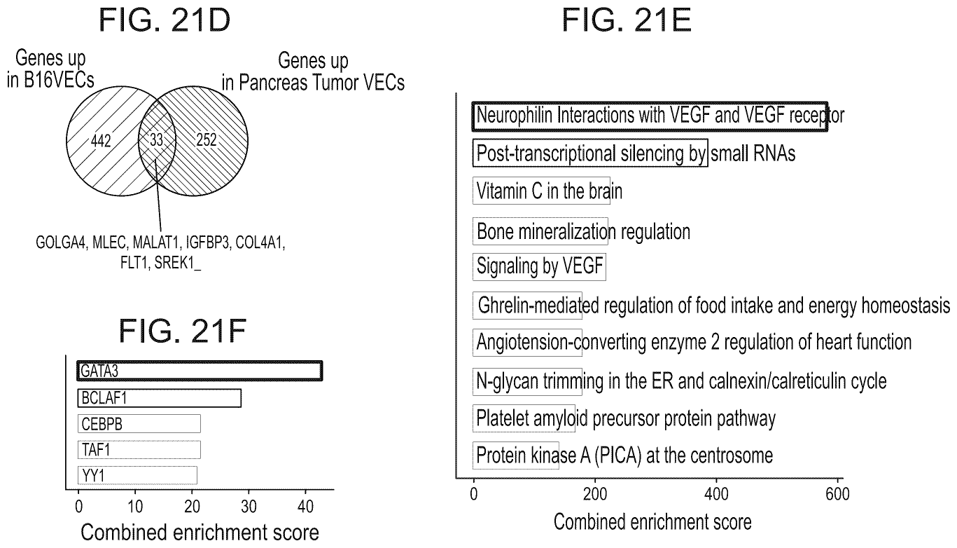

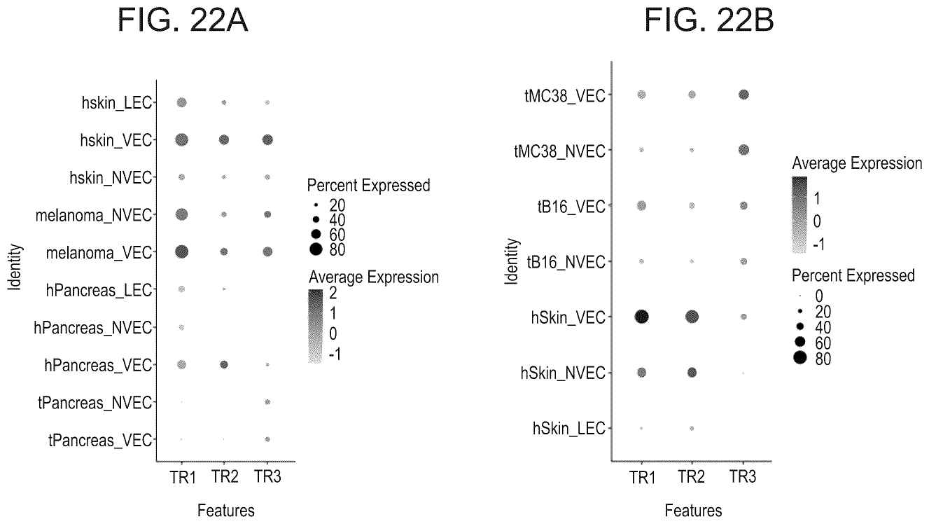

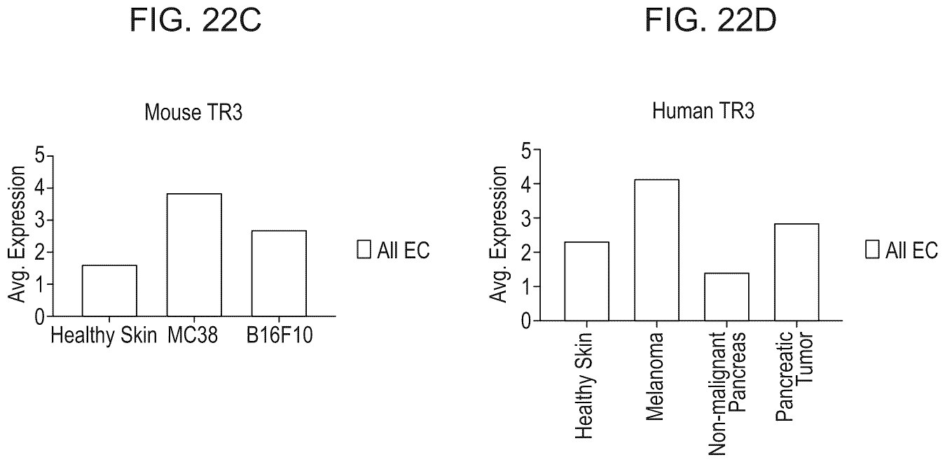

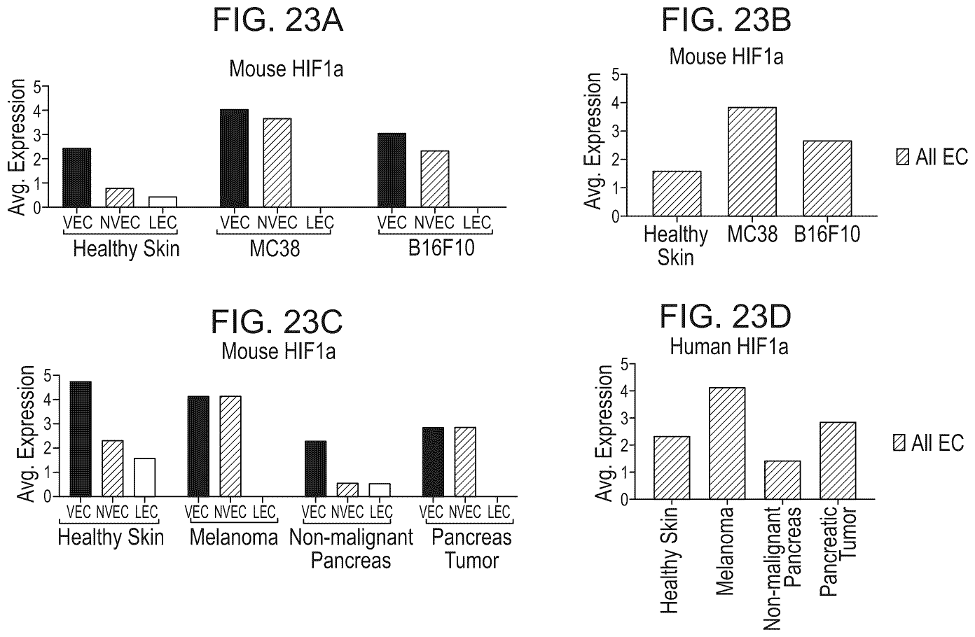

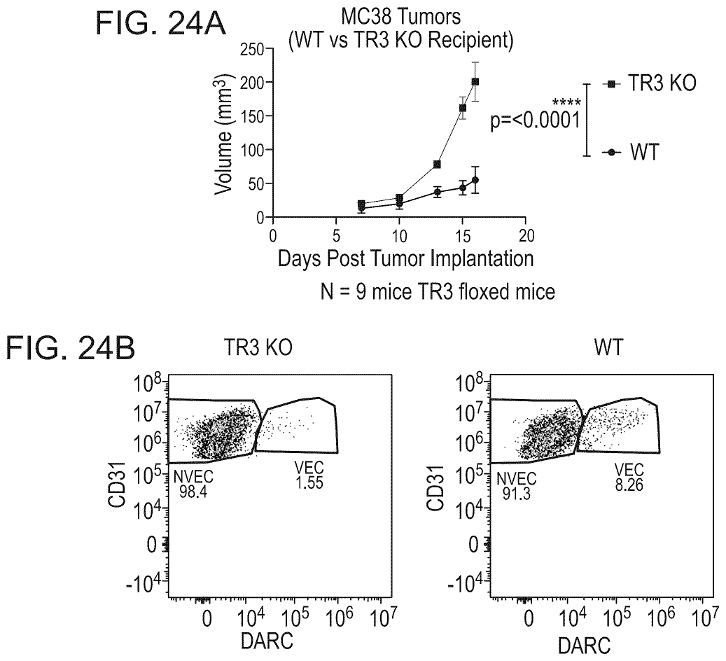

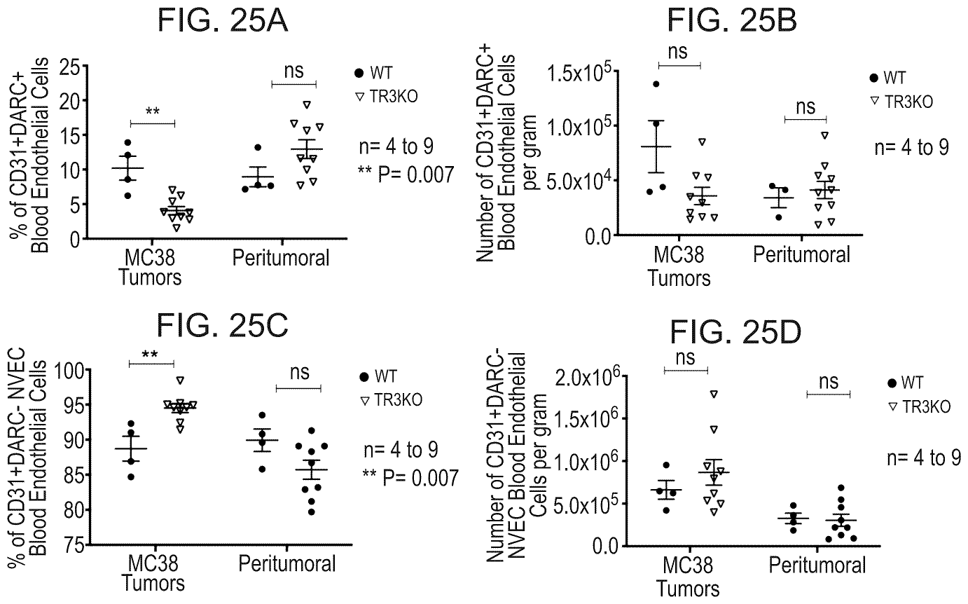

In order to look for differences between immunogenic and non-immunogenic VECs in humans, differential expression between melanoma (immunogenic) VECs and pancreatic tumor (non-immunogenic) VECs was looked at. This gene list was compared to the list of differentially expressed genes in MC38 VECs versus B16 VECs to search for common genes and pathways. 119 genes were found to be upregulated in both mouse and human immunogenic tumors (FIG. 21A) while 33 genes were found to be upregulated in both non-immunogenic tumors (FIG. 21B) (see full lists Table 3). Interestingly in the immunogenic tumor, upregulation was observed of core VEC genes such as DARC, ICAM1 and LRG1. This once again confirm that immunogenicity correlates with a healthier VEC profile. The expression of pro-inflammatory genes and chemokines (JUN, CXCL10), which are known to promote T cell adhesion and recruitment in EC, was observed.^{31,32}

TABLE 3
Shared genes between VECs in human and mouse tumors

| Immunogenic | Non-immunogenic |
|---|---|
| tumors | tumors |
| Vcan | Col4a1 |
| Tgfbr3 | Golga4 |
| Fstl1 | Mlec |
| Ncoa7 | Rbm39 |
| Dtd1 | Nes |
| Rps20 | Hecw2 |
| Mfhas1 | Stc1 |
| Ctss | Ankrd11 |
| Tspan4 | Srsf11 |
| Srp9 | Inpp5a |
| Psmb5 | Cdc42bpb |
| Nup50 | Atad2b |
| Kif26a | Srek1 |
| Ube216 | Evl |
| Psmd1 | Dmtf1 |
| Fos | Malat1 |
| Cst3 | Uaca |
| Fgl2 | Arglu1 |
| Ephb4 | Igfbp3 |
| Ppp1r10 | Apold1 |
| Psen1 | Flt1 |
| Vamp5 | Rbp7 |
| Lrrc16a | Itsn2 |
| B3gnt3 | Pcdh17 |
| Arl4c | Rbm25 |
| Gbp3 | Mlh3 |
| Wars | Tnrc6a |
| Irf1 | Pcnt |
| Txnl1 | Safb |
| Egr1 | Vps53 |
| Sox17 | Akap12 |
| Tuba1b | Eif5b |
| Ugcg | Ppap2a |
| Cd74 | |
| Sod2 | |
| Fcgrt | |
| Clec14a | |
| Srsf4 | |
| Pogk | |
| Syngr2 | |
| Ccni | |
| Zfp3612 | |
| Rgs2 | |
| Samhd1 | |
| Glul | |
| Myc | |
| Rnf19b | |
| Mat2a | |
| Tnfrsf1b | |
| Tsc22d1 | |
| Pcdh19 | |
| Mob4 | |
| Rplp0 | |
| Lamb2 | |
| Kctd10 | |
| Ier2 | |
| Ddx3x | |
| Ppapdc1b | |
| Rpl10a | |
| Utp20 | |
| Cd47 | |
| Anxa5 | |
| Cd9 | |
| Vps29 | |
| Hnrnpa0 | |
| Ifnar1 | |
| Ifitm3 | |
| Socs3 | |
| Pmp22 | |
| Paf1 | |
| Arl6ip1 | |
| Sertad1 | |
| Scarb1 | |
| Csf2rb | |
| Ifi27 | |
| Ppp1r15a | |
| Sav1 | |
| Dpy1914 | |
| Pttg1ip | |
| Fbln2 | |
| Cdc42 | |
| Lrrc8c | |
| Actr2 | |
| Ebf3 | |
| Klf2 | |
| Helz2 | |
| Meox2 | |
| Wdr82 | |
| Cxcl10 | |
| Chka | |
| Thbs1 | |
| Ppm1f | |
| Ctsl | |
| Ablim1 | |
| Lmo4 | |
| Darc | |
| Cdkn1a | |
| Ptgs2 | |
| Ly6e | |
| Jun | |
| Psmb8 | |
| Eef1a1 | |
| Nfkb2 | |
| Dusp1 | |
| Klf4 | |
| Nudt3 | |
| Lrp5 | |
| Fosb | |
| Ctgf | |
| Jak2 | |
| Crim1 | |
| Clu | |
| Imp3 | |
| C2cd2 | |
| Thbd | |
| Junb | |
| Lrg1 | |
| Pds5a | |
| Icam1 | |

In non-immunogenic tumors, expression of IGFBP3 and MALAT1 can be observed, both are known to be upregulated in cancer and are associated with poor prognosis^{33,34} which is consistent with the fact that non-immunogenic tumors are often harder to treat. They're both associated with vascular growth which suggest that the lower T-cell infiltration in non-immunogenic tumor is not due to a lack of neovascularization but to the fact that those VECs are less efficient at capturing T cells and at facilitating their transfer into the tumor microenvironment.

Next, the pathways and transcription factors that might be upregulated in immunogenic tumors were explored. Immunogenicity in a tumor may be improved by turning on relevant pathways. R implementation of EnrichR, a gene enrichment tool which currently contains a large collection of diverse gene set libraries available for analysis and download, was used.^{35,36} In total, EnrichR currently contains 180,184 annotated gene sets from 102 gene set libraries. The BioPlanet database, which integrates pathway annotations from publicly available, manually curated sources that have been subjected to thorough redundancy and consistency cross-evaluation via extensive manual curation, was used to analyze pathways. For transcription factor (TF) analysis the data presented here was generated using the most recent Chip-Seq ENCODE database^{37,38} but other TF databases gave similar results. (enrichment tables in Tables 4-7).

TABLE 4
GSVA pathway enrichment in MC38.

| Name | logFC | AveExpr | P.Value | adj.P.Val |
|---|---|---|---|---|
| GO_TRIGLYCERIDE_CATABOLIC_PROCESS | 0.50 | 0.00 | 0.00 | 0.00 |
| GO_MHC_CLASS_II_PROTEIN_COMPLEX | 0.50 | −0.19 | 0.00 | 0.01 |
| GO_MHC_CLASS_II_PROTEIN_COMPLEX_BINDING | 0.46 | −0.12 | 0.00 | 0.00 |
| REACTOME_REGULATION_OF_IFNA_SIGNALING | 0.40 | −0.23 | 0.00 | 0.00 |
| REACTOME_HDL_MEDIATED_LIPID_TRANSPORT | 0.39 | −0.03 | 0.00 | 0.00 |
| GO_REVERSE_CHOLESTEROL_TRANSPORT | 0.36 | 0.05 | 0.00 | 0.00 |
| RAFFEL_VEGFA_TARGETS_UP | 0.36 | 0.00 | 0.00 | 0.00 |
| GO_ACYLGLYCEROL_CATABOLIC_PROCESS | 0.33 | 0.03 | 0.00 | 0.00 |
| REACTOME_REGULATION_OF_IFNG_SIGNALING | 0.31 | −0.17 | 0.00 | 0.00 |
| WEINMANN_ADAPTATION_TO_HYPOXIA_DN | 0.31 | −0.08 | 0.00 | 0.00 |
| REACTOME_LIPID_DIGESTION_MOBILI- | 0.30 | −0.02 | 0.00 | 0.00 |
| ZATION_AND_TRANSPORT | | | | |
| REACTOME_LIPOPROTEIN_METABOLISM | 0.29 | −0.04 | 0.00 | 0.00 |
| GO_RENAL_SYSTEM_VASCULATURE_DEVELOPMENT | 0.28 | −0.08 | 0.00 | 0.00 |
| WEINMANN_ADAPTATION_TO_HYPOXIA_UP | 0.28 | −0.10 | 0.00 | 0.00 |
| KIM_HYPOXIA | 0.25 | −0.12 | 0.00 | 0.00 |
| GO_CELL_ADHESION_MEDIATED_BY_INTEGRIN | 0.25 | −0.06 | 0.00 | 0.00 |
| BIOCARTA_NFKB_PATHWAY | 0.24 | −0.08 | 0.00 | 0.00 |
| ABE_VEGFA_TARGETS_30 MIN | 0.24 | −0.14 | 0.00 | 0.00 |
| GO_LONG_CHAIN_FATTY_ACID_TRANSPORT | 0.24 | 0.00 | 0.00 | 0.00 |
| GO_STEROL_TRANSPORT | 0.23 | 0.00 | 0.00 | 0.00 |
| HALLMARK_ANGIOGENESIS | 0.23 | −0.09 | 0.00 | 0.00 |
| HAN_JNK_SINGALING_UP | 0.23 | −0.17 | 0.00 | 0.01 |
| GO_POSITIVE_REGULATION_OF_OXIDO- | 0.22 | −0.04 | 0.00 | 0.00 |
| REDUCTASE_ACTIVITY | | | | |
| GO_REGULATION_OF_NITRIC_OXIDE_SYN- | 0.22 | −0.09 | 0.00 | 0.00 |
| THASE_ACTIVITY | | | | |
| FRIDMAN_SENESCENCE_DN | 0.22 | −0.06 | 0.00 | 0.01 |
| GO_ENDOTHELIAL_CELL_PROLIFERATION | 0.21 | −0.10 | 0.00 | 0.00 |
| FRIDMAN_SENESCENCE_UP | 0.21 | −0.13 | 0.00 | 0.00 |
| GO_NEGATIVE_REGULATION_OF_ENDO- | 0.21 | −0.10 | 0.00 | 0.01 |
| THELIAL_CELL_PROLIFERATION | | | | |
| GO_RESPIRATORY_GASEOUS_EXCHANGE | 0.20 | −0.10 | 0.00 | 0.00 |
| MENSSEN_MYC_TARGETS | 0.20 | −0.12 | 0.00 | 0.01 |
| DAUER_STAT3_TARGETS_DN | 0.20 | −0.13 | 0.00 | 0.00 |
| GO_RESPONSE_TO_INTERFERON_BETA | 0.20 | −0.04 | 0.00 | 0.00 |
| GO_ARTERY_MORPHOGENESIS | 0.19 | −0.10 | 0.00 | 0.00 |
| ZHANG_PROLIFERATING_VS_QUIESCENT | 0.19 | −0.14 | 0.00 | 0.01 |
| GO_ANTIGEN_PROCESSING_AND_PRESEN- | 0.19 | −0.12 | 0.00 | 0.01 |
| TATION_OF_EXOGENOUS_PEPTIDE_ANTI- | | | | |
| GEN_VIA_MHC_CLASS_I | | | | |
| DAUER_STAT3_TARGETS_UP | 0.19 | −0.08 | 0.00 | 0.01 |
| GO_POSITIVE_REGULATION_OF_LIPID_BIOSYN- | 0.18 | −0.06 | 0.00 | 0.01 |
| THETIC_PROCESS | | | | |
| GO_FATTY_ACID_TRANSPORT | 0.18 | 0.01 | 0.00 | 0.01 |
| GO_POSITIVE_REGULATION_OF_COAGULATION | 0.18 | −0.03 | 0.00 | 0.00 |
| GO_POSITIVE_REGULATION_OF_ATPASE_ACTIVITY | 0.18 | −0.10 | 0.00 | 0.01 |
| GO_ARTERY_DEVELOPMENT | 0.17 | −0.08 | 0.00 | 0.00 |
| GO_POSITIVE_REGULATION_OF_STEROID_META- | 0.17 | −0.02 | 0.00 | 0.00 |
| BOLIC_PROCESS | | | | |
| GO_REGULATION_OF_ENDOTHELIAL_CELL_PRO- | 0.17 | −0.08 | 0.00 | 0.00 |
| LIFERATION | | | | |
| SANA_TNF_SIGNALING_UP | 0.17 | −0.06 | 0.00 | 0.01 |
| DER_IFN_BETA_RESPONSE_UP | 0.17 | −0.13 | 0.00 | 0.00 |
| GO_ANTIGEN_BINDING | 0.16 | −0.05 | 0.00 | 0.00 |
| PETROVA_PROX1_TARGETS_DN | 0.16 | −0.09 | 0.00 | 0.00 |
| GO_POSITIVE_REGULATION_OF_ENDO- | 0.16 | −0.08 | 0.00 | 0.01 |
| THELIAL_CELL_PROLIFERATION | | | | |
| DER_IFN_ALPHA_RESPONSE_UP | 0.15 | −0.11 | 0.00 | 0.01 |
| GO_POSITIVE_REGULATION_OF_REACTIVE_OXY- | 0.15 | −0.07 | 0.00 | 0.00 |
| GEN_SPECIES_BIOSYNTHETIC_PROCESS | | | | |
| HARRIS_HYPOXIA | 0.15 | −0.06 | 0.00 | 0.01 |
| GO_REGULATION_OF_NITRIC_OXIDE_BIOSYN- | 0.15 | −0.06 | 0.00 | 0.01 |
| THETIC_PROCESS | | | | |
| GO_REGULATION_OF_CGMP_METABOLIC_PROCESS | 0.15 | 0.06 | 0.00 | 0.01 |
| GO_RESPONSE_TO_VIRUS | 0.15 | −0.10 | 0.00 | 0.00 |
| GO_DEFENSE_RESPONSE_TO_VIRUS | 0.14 | −0.09 | 0.00 | 0.00 |
| GO_REGULATION_OF_OXIDOREDUCTASE_ACTIVITY | 0.14 | −0.06 | 0.00 | 0.00 |
| GO_ANTIGEN_PROCESSING_AND_PRESEN- | 0.13 | −0.12 | 0.00 | 0.01 |
| TATION_OF_PEPTIDE_ANTIGEN | | | | |
| GO_AORTA_DEVELOPMENT | 0.13 | −0.07 | 0.00 | 0.01 |
| GO_POSITIVE_REGULATION_OF_REACTIVE_OXY- | 0.12 | −0.07 | 0.00 | 0.01 |
| GEN_SPECIES_METABOLIC_PROCESS | | | | |
| ZHENG_IL22_SIGNALING_UP | 0.11 | 0.03 | 0.00 | 0.01 |
| WINTER_HYPOXIA_METAGENE | 0.11 | −0.09 | 0.00 | 0.01 |
| GO_ORGANIC_HYDROXY_COMPOUND_TRANSPORT | 0.11 | 0.02 | 0.00 | 0.00 |
| GO_REGULATION_OF_REACTIVE_OXYGEN_SPE- | 0.11 | −0.06 | 0.00 | 0.01 |
| CIES_METABOLIC_PROCESS | | | | |
| GO_REGULATION_OF_LIPID_BIOSYN- | 0.10 | −0.02 | 0.00 | 0.01 |
| THETIC_PROCESS | | | | |
| GO_MONOCARBOXYLIC_ACID_TRANSPORT | 0.10 | 0.03 | 0.00 | 0.01 |
| GO_ORGANIC_ANION_TRANSMEMBRANE_TRANS- | −0.10 | 0.11 | 0.00 | 0.01 |
| PORTER_ACTIVITY | | | | |
| GO_AMINO_ACID_TRANSMEMBRANE_TRANS- | −0.12 | 0.10 | 0.00 | 0.01 |
| PORTER_ACTIVITY | | | | |

Adj.P.Val: adjusted p value calculated using the Benjamini-Hochberg procedure.

TABLE 5
GSVA pathway enrichment in B16.

| Name | logFC | AveExpr | P.Value | adj.P.Val |
|---|---|---|---|---|
| GO_MHC_CLASS_II_PROTEIN_COMPLEX_BINDING | 0.44 | −0.12 | 0.00 | 0.00 |
| WEINMANN_ADAPTATION_TO_HYPOXIA_DN | 0.39 | −0.08 | 0.00 | 0.00 |
| WEINMANN_ADAPTATION_TO_HYPOXIA_UP | 0.38 | −0.10 | 0.00 | 0.00 |
| GO_TRIGLYCERIDE_CATABOLIC_PROCESS | 0.34 | 0.00 | 0.00 | 0.01 |
| GO_CELL_ADHESION_MEDIATED_BY_INTEGRIN | 0.29 | −0.06 | 0.00 | 0.00 |
| FRIDMAN_SENESCENCE_DN | 0.29 | −0.06 | 0.00 | 0.00 |
| MENSSEN_MYC_TARGETS | 0.27 | −0.12 | 0.00 | 0.00 |
| ABE_VEGFA_TARGETS_30 MIN | 0.26 | −0.14 | 0.00 | 0.01 |
| KIM_HYPOXIA | 0.24 | −0.12 | 0.00 | 0.01 |
| FRIDMAN_SENESCENCE_UP | 0.21 | −0.13 | 0.00 | 0.00 |
| GO_ARTERY_DEVELOPMENT | 0.18 | −0.08 | 0.00 | 0.00 |
| KARLSSON_TGFB1_TARGETS_UP | 0.18 | −0.12 | 0.00 | 0.00 |
| GO_ARTERY_MORPHOGENESIS | 0.17 | −0.10 | 0.00 | 0.01 |
| GO_AORTA_DEVELOPMENT | 0.14 | −0.07 | 0.00 | 0.01 |
| HAN_JNK_SINGALING_DN | −0.19 | −0.10 | 0.00 | 0.01 |
| GO_SODIUM_INDEPENDENT_ORGANIC_AN- | −0.35 | −0.11 | 0.00 | 0.00 |
| ION_TRANSMEMBRANE_TRANSPORTER_ACTIVITY | | | | |

Adj.P.Val: adjusted p value calculated using the Benjamini-Hochberg procedure.

TABLE 6
EnrichR pathway enrichment in Immunogenic and non-immunogenic mouse and human tumors.

| | | | | | |
|---|---|---|---|---|---|
| Term | Overlap | P.value | Adj.P.Val | CS | Genes |

IMMUNOGENIC

| | | | | | |
|---|---|---|---|---|---|
| TSP1-induced apoptosis in | 3/8 | 1.13E−05 | 0.000894 | 718 | JUN; FOS; THBS1 |
| microvascular endothelial cell | | | | | |
| Erythropoietin-mediated | 3/11 | 3.27E−05 | 0.001765 | 473 | CDKN1A; JAK2; |
| neuroprotection through NF-kB | | | | | SOD2 |
| Interleukin-6 signaling | 8/71 | 9.64E−09 | 2.08E−06 | 350 | SOCS3; JUN; LMO4; |
| pathway | | | | | MYC; IRF1; FOS; |
| | | | | | JAK2; JUNB |
| Interleukin-11 pathway | 4/23 | 9.67E−06 | 0.000859 | 338 | SOCS3; JUN; |
| | | | | | FOS; ICAM1 |
| Regulation of NFAT | 6/47 | 3.44E−07 | 3.71E−05 | 319 | EGR1; JUN; FOS; |
| transcription factors | | | | | PTGS2; JUNB; GBP3 |
| Inhibition of cellular | 4/24 | 1.15E−05 | 0.000872 | 318 | JUN; MYC; |
| proliferation by Gleevec | | | | | FOS; JAK2 |
| Interferon alpha/beta signaling | 7/64 | 1.04E−07 | 1.74E−05 | 296 | IFITM3; SOCS3; EGR1; |
| | | | | | IFI27; IRF1; PSMB8; |
| | | | | | IFNAR1 |
| Cadmium-induced DNA | 3/17 | 0.00013 | 0.005222 | 265 | JUN; MYC; FOS |
| biosynthesis and proliferation | | | | | |
| in macrophages | | | | | |
| Interferon signaling | 12/168 | 3.62E−10 | 1.82E−07 | 261 | IFITM3; SOCS3; EGR1; |
| | | | | | IFI27; NUP50; IRF1; |
| | | | | | UBE2L6; JAK2; PSMB8; |
| | | | | | ICAM1; IFNAR1; GBP3 |
| AP-1 transcription factor | 7/70 | 1.94E−07 | 2.44E−05 | 260 | EGR1; JUN; DUSP1; |
| network | | | | | MYC; FOSB; FOS; JUNB |
| T cell receptor calcium | 4/29 | 2.52E−05 | 0.001523 | 245 | JUN; FOS; PTGS2; |
| pathway | | | | | JUNB |
| BDNF signaling pathway | 15/261 | 4.48E−11 | 3.38E−08 | 230 | EGR1; JUN; CDKN1A; |
| | | | | | LMO4; DUSP1; FOS; |
| | | | | | PTGS2; CLU; RGS2; |
| | | | | | VCAN; ABLIM1; MYC; |
| | | | | | FOSB; JUNB; IER2 |
| TSH regulation of gene | 8/97 | 1.15E−07 | 1.74E−05 | 221 | PPP1R15A; EGR1; |
| expression | | | | | RGS2; JUN; MYC; |
| | | | | | FOS; PTGS2; ICAM1 |
| Activation of the AP-1 family | 2/10 | 0.00153 | 0.027202 | 218 | JUN; FOS |
| of transcription factors | | | | | |
| ID regulation of gene | 3/20 | 0.00022 | 0.006841 | 213 | CDKN1A; THBS1; |
| expression | | | | | ICAM1 |
| T cell receptor regulation of | 24/603 | 1.21E−13 | 1.83E−10 | 199 | IFITM3; EGR1; JUN; |
| apoptosis | | | | | CDKN1A; DUSP1; |
| | | | | | POGK; CSF2RB; FOS; |
| | | | | | RPL10A; TNFRSF1B; |
| | | | | | SOD2; CLU; PSMB8; |
| | | | | | CDC42; CXCL10; |
| | | | | | FCGRT; MYC; IRF1; |
| | | | | | FOSB; RPS20; JUNB; |
| | | | | | IER2; IFNAR1; GBP3 |
| Interleukin-5 signaling | 5/49 | 1.06E−05 | 0.000889 | 196 | JUN; MYC; CSF2RB; |
| pathway | | | | | FOS; JAK2 |
| Type II interferon signaling | 5/50 | 1.17E−05 | 0.000843 | 191 | SOCS3; CXCL10; IRF1; |
| (interferon-gamma) | | | | | JAK2; ICAM1 |
| Interleukin-4 regulation of | 14/267 | 6.89E−10 | 2.60E−07 | 186 | JUN; CDKN1A; FGL2; |
| apoptosis | | | | | RNF19B; UBE2L6; |
| | | | | | CSF2RB; FOS; PTGS2; |
| | | | | | ARL4C; RGS2; VCAN; |
| | | | | | CTSL; MYC; CD9 |
| Signaling events mediated by | 3/23 | 0.00033 | 0.009683 | 176 | EGR1; CDKN1A; |
| PRL | | | | | TUBA1B |

NON-IMMUNOGENIC

| | | | | | |
|---|---|---|---|---|---|
| Neurophilin interactions with | 1/5 | 0.00822 | 1 | 582 | FLT1 |
| VEGF and VEGF receptor | | | | | |
| Post-transcriptional silencing | 1/7 | 0.01149 | 1 | 387 | TNRC6A |
| by small RNAs | | | | | |
| Vitamin C in the brain | 1/11 | 0.01801 | 1 | 221 | COL4A1 |
| Bone mineralization regulation | 1/11 | 0.01801 | 1 | 221 | COL4A1 |
| Signaling by VEGF | 1/11 | 0.01801 | 1 | 221 | FLT1 |
| Ghrelin-mediated regulation of | 1/13 | 0.02125 | 1 | 180 | IGFBP3 |
| food intake and energy | | | | | |
| homeostasis | | | | | |
| Angiotensin-converting | 1/13 | 0.02125 | 1 | 180 | COL4A1 |
| enzyme 2 regulation of heart | | | | | |
| function | | | | | |
| N-glycan trimming in the ER | 1/13 | 0.02125 | 1 | 180 | MLEC |
| and calnexin/calreticulin cycle | | | | | |
| Platelet amyloid precursor | 1/14 | 0.02286 | 1 | 164 | COL4A1 |
| protein pathway | | | | | |
| Protein kinase A (PKA) at the | 1/16 | 0.02609 | 1 | 138 | PCNT |
| centrosome | | | | | |
| Insulin-like growth factor | 1/17 | 0.02769 | 1 | 128 | IGFBP3 |
| (IGF) activity regulation by | | | | | |
| insulin-like growth factor | | | | | |
| binding proteins (IGFBPs) | | | | | |
| Eukaryotic protein translation | 1/17 | 0.02769 | 1 | 128 | EIF5B |
| Acute myocardial infarction | 1/20 | 0.0325 | 1 | 104 | COL4A1 |
| Mismatch repair | 1/23 | 0.03729 | 1 | 87 | MLH3 |
| Angiogenesis | 1/23 | 0.03729 | 1 | 87 | FLT1 |
| Hypoxia and p53 in the | 1/23 | 0.03729 | 1 | 87 | IGFBP3 |
| cardiovascular system | | | | | |
| Triacylglyceride biosynthesis | 1/24 | 0.03888 | 1 | 82 | PPAP2A |
| Regulatory RNA pathways | 1/25 | 0.04047 | 1 | 78 | TNRC6A |
| VEGFR1 pathway | 1/27 | 0.04363 | 1 | 70 | FLT1 |
| S1P/S1P3 pathway | 1/29 | 0.04679 | 1 | 64 | FLT1 |

Overlap: overlap between the input set of genes and the annotated gene sets. Adj.P.Val: adjusted p value calculated using the Benjamini-Hochberg procedure. CS: combined score. Combination of the p-value and z-score calculated by multiplying the two scores as follows: c = ln(p) * z. Where c is the combined score, p is the p-value computed using Fisher's exact test, and z is the z-score computed to assess the deviation from the expected rank.

TABLE 7
EnrichR TF enrichment in Immunogenic and non-immunogenic mouse and human tumors.

| | | | | | |
|---|---|---|---|---|---|
| Term | Overlap | P.value | Adj.P.Val | CS | Genes |

IMMUNOGENIC

| | | | | | |
|---|---|---|---|---|---|
| STAT1 | 30/945 | 2.46E−14 | 2.01E−11 | 167 | IFITM3; CDKN1A; LRP5; UBE2L6; RPL10A; |
| HeLa-S3 | | | | | PTGS2; NUDT3; CTSS; ICAM1; SOCS3; |
| hg19 | | | | | ABLIM1; SYNGR2; TSPAN4; JAK2; JUNB; |
| | | | | | EGR1; JUN; WARS; FOS; TNFRSF1B; SOD2; |
| | | | | | PSMB8; NFKB2; EEF1A1; LRG1; IFI27; |
| | | | | | IRF1; NCOA7; LY6E; IFNAR1 |
| RELA | 26/1302 | 3.72E−08 | 6.08E−06 | 57 | CDKN1A; DDX3X; CCNI; ARL6IP1; RPL10A; |
| GM12892 | | | | | ZFP36L2; ICAM1; SYNGR2; MAT2A; JAK2; |
| hg19 | | | | | GLUL; JUNB; IER2; HNRNPA0; CD74; |
| | | | | | JUN; ANXA5; TNFRSF1B; PSMB8; NFKB2; |
| | | | | | EEF1A1; MOB4; LRG1; DPY19L4; IRF1; |
| | | | | | IFNAR1 |
| STAT2 | 9/271 | 3.56E−05 | 0.001383 | 57 | IFITM3; DDX3X; WARS; IFI27; IRF1; |
| K562 hg19 | | | | | UBE2L6; NCOA7; SAMHD1; ICAM1 |
| SMC3 | 34/2000 | 1.03E−08 | 2.80E−06 | 53 | CDKN1A; DDX3X; C2CD2; RPLP0; FGL2; |
| CH12.LX | | | | | RPL10A; CLU; PPM1F; SAV1; CST3; |
| mm9 | | | | | SYNGR2; JAK2; GLUL; PPAPDC1B; DTD1; |
| | | | | | CHKA; LMO4; DUSP1; ANXA5; TNFRSF1B; |
| | | | | | SRP9; PSMB8; NFKB2; EEF1A1; TGFBR3; |
| | | | | | ARL4C; MFHAS1; DPY19L4; IRF1; PAF1; |
| | | | | | CD9; RPS20; CD47; IFNAR1 |
| UBTF | 30/2000 | 1.43E−06 | 0.00013 | 34 | VPS29; CCNI; FGL2; PSEN1; PDS5A; |
| CH12.LX | | | | | ZFP36L2; SAV1; SOCS3; JAK2; PPAPDC1B; |
| mm9 | | | | | DTD1; EPHB4; HNRNPA0; ACTR2; DUSP1; |
| | | | | | KLF4; PSMB8; NFKB2; MOB4; ARL4C; |
| | | | | | KCTD10; DPY19L4; NUP50; IRF1; PAF1; |
| | | | | | RPS20; NCOA7; CD47; VAMP5; IFNAR1 |
| CHD1 | 14/691 | 6.29E−05 | 0.001975 | 33 | DDX3X; PPP1R10; FOS; RPL10A; SOD2; |
| MEL cell | | | | | ZFP36L2; EEF1A1; MAT2A; MYC; TSPAN4; |
| line mm9 | | | | | CD47; JUNB; VAMP5; HNRNPA0 |
| STAT5A | 6/217 | 0.00191 | 0.019008 | 29 | PPP1R15A; SOCS3; WARS; LRG1; IMP3; |
| K562 hg19 | | | | | SRSF4 |
| STAT3 | 28/1974 | 1.03E−05 | 0.000599 | 27 | IFITM3; CCNI; ARL6IP1; UBE2L6; PTGS2; |
| HeLa-S3 | | | | | FSTL1; CTGF; ICAM1; UGCG; SOCS3; |
| hg19 | | | | | ABLIM1; MYC; JUNB; GBP3; EGR1; JUN; |
| | | | | | WARS; CHKA; IMP3; DUSP1; CRIM1; FOS; |
| | | | | | SOD2; PSMB8; IRF1; NCOA7; VAMP5; |
| | | | | | IFNAR1 |
| CEBPB | 23/1513 | 2.56E−05 | 0.001099 | 27 | IFITM3; CHKA; LMO4; PPP1R10; PSEN1; |
| C2C12 | | | | | RPL10A; MEOX2; TNFRSF1B; PTGS2; |
| mm9 | | | | | SAMHD1; FSTL1; PPM1F; MOB4; SYNGR2; |
| | | | | | WDR82; CTSL; DPY19L4; MYC; CD9; |
| | | | | | RPS20; CD47; JUNB; IER2 |
| ATF3 | 28/1991 | 1.21E−05 | 0.000656 | 27 | VPS29; CCNI; ARL6IP1; RPLP0; RPL10A; |
| A549 hg19 | | | | | PTGS2; SAMHD1; CLU; SAV1; SOCS3; |
| | | | | | THBD; SERTAD1; MYC; TSPAN4; PSMD1; |
| | | | | | JUNB; HNRNPA0; ACTR2; JUN; IMP3; |
| | | | | | SRP9; EEF1A1; MOB4; KCTD10; DPY19L4; |
| | | | | | PAF1; FOSB; RPS20 |

NON-IMMUNOGENIC

| | | | | | |
|---|---|---|---|---|---|
| GATA3 | 9/1185 | 9.22E−05 | 0.037614 | 43 | RBM39; IGFBP3; HECW2; STC1; MALAT1; |
| SK-N-SH | | | | | UACA; APOLD1; PCDH17; MLH3 |
| hg19 | | | | | |
| BCLAF1 | 7/1007 | 0.00108 | 0.126471 | 29 | EIF5B; RBM25; DMTF1; MLEC; MALAT1; |
| K562 hg19 | | | | | SREK1; ATAD2B |
| CEBPB | 7/1173 | 0.00261 | 0.177375 | 22 | RBM39; EIF5B; ANKRD11; STC1; MALAT1; |
| MCF-7 | | | | | SREK1; APOLD1 |
| hg19 | | | | | |
| TAF1 | 9/1695 | 0.00131 | 0.118625 | 21 | RBM39; RBM25; GOLGA4; DMTF1; STC1; |
| MCF-7 | | | | | SREK1; PCNT; UACA; ATAD2B |
| hg19 | | | | | |
| YY1 | 10/2000 | 0.00105 | 0.17073 | 21 | RBM39; EIF5B; RBM25; INPP5A; VPS53; |
| GM12878 | | | | | EVL; MALAT1; CDC42BPB; SREK1; SRSF11 |
| hg19 | | | | | |
| SP2 | 8/1507 | 0.00258 | 0.191335 | 19 | RBM39; EIF5B; RBM25; ANKRD11; VPS53; |
| HepG2 | | | | | PCNT; UACA; ATAD2B |
| hg19 | | | | | |
| EP300 | 6/1155 | 0.01059 | 0.375767 | 14 | RBM39; RBM25; DMTF1; MALAT1; |
| T47D hg19 | | | | | TNRC6A; ATAD2B |
| SP1 K562 | 6/1249 | 0.01518 | 0.364235 | 12 | RBM39; ANKRD11; MLEC; MALAT1; |
| hg19 | | | | | PCNT; APOLD1 |
| MEF2A | 5/970 | 0.02038 | 0.426409 | 12 | DMTF1; MLEC; MALAT1; APOLD1; |
| GM12878 | | | | | ATAD2B |
| hg19 | | | | | |
| EGR1 | 4/741 | 0.03264 | 0.532647 | 11 | RBM39; RBM25; MALAT1; UACA |
| MCF-7 | | | | | |
| hg19 | | | | | |

Overlap: overlap between the input set of genes and the annotated gene sets. Adj.P.Val: adjusted p value calculated using the Benjamini-Hochberg procedure. CS: combined score. Combination of the p-value and z-score calculated by multiplying the two scores as follows: c = In(p) * z. Where c is the combined score, p is the p-value computed using Fisher's exact test, and z is the z-score computed to assess the deviation from the expected rank.

Similarly, to what was observed in mouse data alone, the combined human and mice immunogenic data showed and enrichment for inflammatory pathways (interferon, NF-κb, interleukin, RELA) (FIG. 21C). Down regulation of EC proliferation pathways was observed, which, once again, highlight the importance of proper EC function versus out of control neovascularization. Interestingly many TF involved in immune surveillance and T cell recruitment are upregulated (FIG. 21E) such as the STAT TFs which are intracellular TF that mediate many aspects of cellular immunity, proliferation, apoptosis and differentiation through interferon signaling. Defects in STAT signaling can lead to increased susceptibility to infections while overexpression has been linked to autoimmune diseases. This highlights their key role in immune recruitment.³⁹

Meanwhile, in non-immunogenic tumors an enrichment was observed for endothelial proliferation pathways (VEGF, Angiotensin) (FIG. 21D). Similarly, expression of BCLAF1 and CEBPB have been shown to promote angiogenesis by controlling the expression of the hypoxia inducible factor-1α (HIF-1α) (FIG. 21F).^{40,41} The presence of GATA3 is also of particular interest as GATA3 has been linked to the inhibition of Ang-1-Tie2 signaling, thus contributing to endothelial cell dysfunction.⁴² The upregulation of post-transcriptional silencing by small RNAs also suggest a disruption of proper VEC function.

Finally, it is of interest to note that EGR1 and ZFP36 are both upregulated in immunogenic tumors VECs even though they have diametrically opposed effects. EGR1 has been well documented as a key mediator to induce the expression of cytokines and growth factors. EGR1 targets genes related to inflammation in vasculature, more specifically TECK and IP-30.⁴³ TECK is a CC chemokine that functions as a chemoattractant for lymphocytes⁴⁴ and IP-30 was originally cloned as an interferon-regulated protein and suggested as playing an important role in IFN-induced inflammation.⁴⁵ Meanwhile ZFP36 has been shown to control inflammation in EC by inhibiting the expression of pro-inflammatory mRNA transcripts.⁴⁶ Those opposing effects could be a key factor is explaining why immunogenic VECs are so efficient at T cell recruitment since ZFP36 could help keep inflammation under control while EGR1 enhances T cell recruitment. ZFP36 and EGR1 could therefore present great targets for induction in non-immunogenic VECs to increase T cell recruitment.

Monoclonal antibodies that specifically recognize DARC (Duffy Antigen Receptor for Chemokines, a.k.a. ACRK1 or CD234) were used to study the distribution of VEC and T cells in healthy and diseased tissues. (See Thiriot et al.) More specifically, the DARC antibodies were used to elucidate the differences between VECs in immunogenic and non-immunogenic tumors environment as VECs regulate T cell infiltration in tissue.

First, a significant correlation between the number VEC and the number of CD8+ T cell in both immunogenic and non-immunogenic tumors in two mouse models, MC38 (immunogenic) and B16 (non-immunogenic) was established. This observation was further confirmed in human melanoma and pancreatic tumors. Furthermore, it became obvious that the number of VEC, and consequently T cells, was much lower in non-immunogenic tumors. Thus, the transcriptional differences between VECs in several tumor microenvironments were determined.

Using SeqWell, a microwell based scRNA-Seq technology,¹⁵ isolated ECs from healthy and tumor samples in both mouse and human were sequenced. After validating the protocol in healthy cells by controlling that usual EC expression patterns could be observed, this platform was used to compare MC38 and B16 VECs. This comparison revealed that (1) regardless of the microenvironment tumor, VECs upregulated hypoxia, angiogenesis and proliferation pathways, (2) immunogenic VECs closely resembled healthy VEC and (3) inflammation pathways (interferon and virus response) were upregulated in immunogenic VECs, thereby promoting T cell recruitment.

Finally, human melanoma (immunogenic) and pancreatic tumor (non-immunogenic) samples in combination with the mouse models were used to identify a common transcriptional signature for immunogenic VECs. They appear to be characterized by the upregulation of STAT and EGR1 transcription factor, which both promote T cell recruitment through the expression of specialized chemokines.^{39,43,44} Immunogenic VECs also express high levels of ZFP36, which might help keep inflammation under control while maintaining active T cell recruitment. This data suggests that these transcription factors could be targeted in non-immunogenic VECs to improve T cell infiltration in those tumors.

These data provide a snapshot of the anti-tumoral immune response, which is also determined by other factors such as intratumoral T cell proliferation, survival and egress into draining lymphatics.

### Methods

### Tumor Implantation and Harvest

Tumor cells were cultured in DMEM supplemented with 10% FBS, 1% of glutamine, penicillin/streptomycin, HEPES (1M stock), sodium pyruvate and non-essential amino acids unless cells were about 75% to 80% confluent. Cell suspension for tumor implantation were prepared at al density of 1×10\hat{ }6 cells per 50 ul for MC38 and B16F10 and 1×10\hat{ }5 cells and 1×10\hat{ }5 cells per 30 ul for murine pancreatic tumor models. MC38 and B16F10 tumors were implanted subcutaneously in the dorsal area of the mouse. KPC and Panc02 tumors were implanted orthotopically in the pancreatic tail by making a small incision and injecting 30 ul in the extravasated pancreas. The pancreas is returned into place and the skin sutured together. All tumors were implanted in C57Bl6J mice and in RAG KO for functional studies, these mice were purchased from Jackson Laboratories. All tumors harvested were about 100 to 200 mg. The peritumoral tissue were careful removed, tumors, peritumoral tissue and healthy tissue from non-tumor bearing mouse were analyzed separately.

### Flow Cytometry and Cell Preparation for Single Cell RNA Sequencing

### Digestion:

Harvested tissue were minced and incubated in digestion media containing a combination of collagenase, DNase, dispase and hyaluronidase with 2% of fetal bovine serum. Sample were incubated on an orbital shaker at 37 C for 10 to 30 mins depending on the density of the supernatant which was transferred to a secondary tube on ice containing enriched media. Undigested tissues bits were put through another digestion cycle for 10 to 20 mins and this process was repeated until all tissue were digestion.

### Single Cell RNA Seq Samples for Seq Well:

Non-enriched samples were transferred directly onto arrays for sequencing. For cell enrichment, CD45 positive cells were depleted by staining the samples with biotinylated anti-CD45 antibody followed by incubation with Dynabeads Biotin Binder (Cat #11047) as per manufacturers protocol. The supernatant was stained with anti-CD31 followed by incubation with Dynabeads, the supernatant were discarded and beads-bounded cells were washed and loaded only seq well arrays for sequencing.

### Flow Cytometry Analysis

Cells were treated with Fc block followed by staining with either mouse or human antibodies for immune cell and endothelial cell profiling. Anti-Ter119 (for mouse) or anti-CD235a/b (for human) antibodies were used to gate out red blood cells. Samples were also stained with antibodies against CD45, CD11b, CD11c, CD31, gp38, DARC, CD3, CD8b and CD4. For endothelial cell subsets, CD45+ter119+ (or CD235a/b) cells were gated out and CD31+gp38-cells were selected for blood endothelial cells (BEC) followed by gating on CD31+ and DARC+ for the venular endothelial cell (VEC) subset and CD31+DARC-non-venular endothelial cell subsets (NVEC) as described in Thiriot et al. BMC biology (10). For CD8 T cell gating, CD45+CD3+CD4−CD8b+ T cells were selected.

### Functional Adoptive Cell Transfer Experiment

Tumors were implanted in RAG KO mice and harvested as described above. Splenocytes from b-actin GFP mice were harvested and differentiated into effector CD8 T cells as described in (Weninger et al. JEM 2001). 5×10\hat{ }6 GFP+CD621−CD8b+CD44+ cells were transferred via retro-orbital route in RAG KO mice bearing tumors. Four hours after transfer, tumors were harvested, digested and prepared for flow cytometric analysis as describe above and by gating on CD45+CD3+GFP+CD8b+ T cells, T cell homing in tumors was assessed.

### Single-Cell Transcriptional Profiling

High-throughput single-cell mRNA sequencing by Seq-Well was per-formed on the single-cell suspensions described above, as previously described. Approximately 20,000 viable cells per sample were applied directly to the surface of a Seq-Well device. Depending on sample sizes 1, 2, 3 or 4 arrays were run for each sample.

### Sequencing and Alignment

Sequencing for all samples was performed either on an Illumina NovaSeq or an Illumina NextSeq. No batch effect related to the type of sequencer used was observed. Reads were aligned to the Mus musculus genome (mm10) or the Homo sapiens (hg19) genome using STAR2, and the aligned reads were then collapsed by cell barcode and unique molecular identifier (UMI) sequences using DropSeq Tools v.1 to generate digital gene expression (DGE) matrices, as previously described^{1,3}. To account for potential index swapping, all cell barcodes from the same sequencing run that were within a hamming distance of 1 were merged.

### Analysis of Single-Cell Sequencing Data

For each array, the quality of constructed libraries was assessed by examining the distribution of reads, genes and transcripts per cell. For each sample, dimensionality reduction (PCA) and clustering was performed as previously described^{4,5}. Results were visualized in a two-dimensional space using UMAP6, and annotated each cluster based on the identity of highly expressed genes. Prior to any further analysis doublets were removed from each sample using DoubletFinder as previously described.⁷ Normalization, RNA counts regression, clustering and further gene analysis such as violin plots, heatmaps, module scoring, differential expression . . . etc. were performed using Seurat tools unless specified otherwise.⁸ In some cases cluster identity was further assessed using module scoring. The gene lists used to define the modules are in Table 2. After EC isolation, to further characterize substructure within EC, dimensionality reduction (PCA) and clustering over those cells alone were performed as previously described¹. Results in two-dimensional space using UMAP were visualized. Clusters were further annotated (that is, as endothelial cells subsets, such as venular cells, capillary cells . . . etc) by cross-referencing cluster-defining genes with curated gene lists or by using module scoring.

### B16 VEC Isolation Strategy

For most samples, a VEC cluster was identified. However, this wasn't the case for B16 as B16 VECs were too similar to B16 NVECs. To identify VECs, VEC scoring based on the gene list in Table 2 with a 0.2 cutoff value was used. To define a score cutoff the B16 data matrix was shuffled and randomized first, then a sample of 100 cells was taken, their VEC score was calculated and the score value for the 95 quantiles was recorded. This process was repeated 10 times over 50 different permutations of the data matrix. The 95 quantiles value was averaged over all these iterations and used as the cutoff.

### Silhouette Analysis

Silhouette⁹ can be used to define the proximity between clusters by scoring the similarity between cells of those clusters. Here for each cell in a given cluster the silhouette algorithm was used to define a closest neighbor cluster (i.e. the cluster the cell should belong to if it didn't belong to the cluster it is currently in). Similarities between clusters were assessed by looking at the percentage of cells from one cluster reassigned to other clusters.

### GSVA Analysis

Gene Set Variation Analysis (GSVA)¹⁰ was used by using a collection of expert annotated vascular-related gene sets¹¹ from the Molecular Signatures Database (MSigDB version 5.2) to identify pathways and cellular processes enriched in different samples. GSVA was performed as implemented in the GSVA R-package (default parameters), where the gene-by-cell matrix is converted into a gene-set-by-cell matrix. The difference between the GSVA enrichment scores from each sample was analyzed by using a simple linear model and moderated t-statistics computed by the limma package using an empirical Bayes shrinkage method.¹² Using a p=0.01 cutoff, the differentially activated pathways between samples were examined.

### EnrichR Analysis

Genes differentially expressed between MC38 VECs and B16 VECs or melanoma VECs and pancreatic tumor VECs were determined using Seurat, as described above. These lists were compared to look for common elements. The resulting list of common elements was used for further gene set analysis with EnrichR. The EnrichR R package provides an interface to the EnrichR database.^{13,14} Two databases were selected: “BioPlanet_2019” for pathways and “ENCODE_TF_ChIP-seq_2015” for transcription factors and looked for enrichment. Full enrichment tables in Tables 6 and 7. The transcription factor analysis was repeated using “ChEA_2016”, “ARCHS4_TFs_Coexp”, “ENCODE_and_ChEA_Consensus_TFs_from_ChIP-X”, “Enrichr_Submissions_TF-Gene_Coocurrence”, and “TRRUST_Transcription_Factors_2019” databases, similar enrichment were observed.

### Example 2: Characterize Expression of Transmembrane Proteins on Microvascular ECs of Solid Tumors

A hallmark of solid tumors is the formation of new vasculature (angiogenesis). This process is required to support tumor growths beyond a few millimeters in size due to the limit of oxygen and nutrient diffusion within neoplastic tissues (Folkman, J. 1971. N Engl J Med 285:1182-1186). Tumor neovasculature is often poorly adhesive for blood-borne T cells, which is thought to present a major impediment to T cell dependent immunotherapy (Peske J D, Woods A B, Engelhard V H. Adv Cancer Res. 2015; 128:263-307). Normal microvasculature, which consists of a network of functionally specialized vessels, including arteries, arterioles, venules and veins, which are all connected by a common capillary network was observed. Arteries and arterioles regulate blood flow, while gas and nutrient exchange takes place at the capillary level. Using intravital microscopy, it has been previously shown that the recruitment of blood-borne leukocytes is invariably restricted to postcapillary and collecting venules, whereas capillaries and arterioles do not support leukocyte adhesion (Halin, C., J. Rodrigo Mora, C. Sumen, and U. H. von Andrian. 2005. Annu Rev Cell Dev Biol 21:581-603). There is strong evidence suggesting that this functional distinction among microvessels is due to segmental specialization of endothelial cells (ECs), not hemodynamic differences (Ley, K., and P. Gachtgens. 1991. Circ Res 69:1034-1041). Indeed, microvascular specialization is already apparent during embryogenesis before the initiation of blood flow (Lawson, N. D., and B. M. Weinstein. 2002. Nat Rev Genet 3:674-682). Both in uninflamed microvessels that constitutively recruit leukocytes, and in acutely or chronically inflamed peripheral tissues, venules are the exclusive port of exit for blood-borne leukocytes that access the extravascular compartment. Accordingly, multiple studies have shown that most leukocyte adhesion receptors are restricted to venular ECs (V-ECs), although the expression of these molecules is not uniform in different vascular beds (von Andrian, U. H., and C. R. Mackay. 2000. N. Engl. J. Med. 343:1020-1034). Several molecules have been identified that specify the differentiation of blood ECs and lymphatic ECs (LECs) and contribute to EC proliferation in tumors (Rocha, S. F., and R. H. Adams. 2009. Angiogenesis 12:139-147; Oliver, G., and R. S. Srinivasan. 2010. Development 137:363-372), but the mechanism(s) that render(s) V-ECs uniquely capable of supporting leukocyte trafficking remain(s) a mystery.

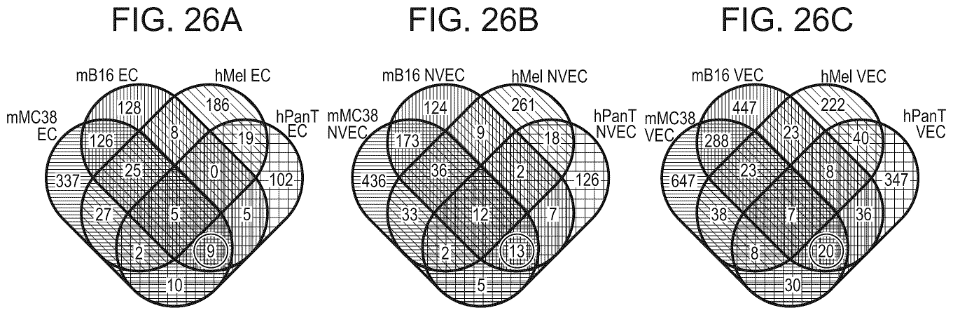

To address this issue, a monoclonal antibody (mAb) against DARC (ACKR1), which selectively recognizes V-ECs in normal murine tissues was used (Thiriot, A. et al. BMC Biology, 2017 May 19; 15 (1): 45). In recent experiments, using this mAb as well as a commercial mAb against human DARC, primary V-ECs, non-venular ECs (NV-ECs) and L-ECs were isolated from a variety of murine and human non-malignant tissues to compare EC subsets at the transcriptome and proteome level. In addition, single-cell RNAseq was used to compare EC transcriptomes of two subcutaneous murine tumors (MC38 colorectal adenocarcinoma and B16F10 melanoma) and fresh patient-derived human melanoma and pancreatic cancer. For each tumor, V-ECs and NV-ECs from peri-tumoral non-malignant tissue were analyzed. MC38 was used because it is an immunogenic tumor (T-cell rich and respond to checkpoint blockade) and B16F10 melanoma because it is a non-immunogenic (T-cell poor and do not respond to checkpoint blockade). Several endothelial genes, including genes encoding cell surface molecules that are uniquely upregulated in the tumor microvasculature were identified (FIGS. 26A-26C).

The mechanisms that enable venular ECs to recruit leukocytes but prohibit capillary and arteriolar endothelium to do so are entirely unknown. The differences between the venular phenotype of an immunogenic tumor and a non-immunogenic tumor is also not known. Identifying gene products that specify endothelial “venuleness” represent a novel class of attractive targets for tumor-specific EC targets and venular inducers for onco-immunotherapy. The only current treatments targeting tumor vasculature aim to inhibit angiogenesis by targeting VEGF, but this approach does not promote venular differentiation. There are no FDA-approved anti-tumoral drugs that can selectively boost endothelial cell dependent immune cell recruitment. This invention emerges from a proprietary discovery platform to generate novel anti-tumoral therapy that aims to differentiate non-adhesive endothelium within tumors into venular endothelium and identify endothelial cells (EC) specific surface markers for targeted treatments of solid tumors with minimum off target effects. Since V-ECs are the principal gatekeepers for leukocyte emigration, drugs that are able to target the intra-tumoral venular segment can be used to promote VEC differentiation could potentially boost tumor infiltration by T cells and thus enhance onco-immunotherapy. Therefore, the neovasculature of solid tumors may be inherently suboptimal at recruiting T cells because of inadequate endothelial differentiation into functional venular type microvessels. The present invention disclosure provides a proprietary discovery platform that will lead to a new generation of drugs that specifically target clinically relevant plasma membrane molecules from venular and non-venular endothelium, in both murine and human solid tumors for the targeted delivery of therapeutics with minimum off target effects. Additionally, it reveals the transcription programming of venular endothelial cells from immunogenic tumors (which are poised for immune cells recruitment), the molecules identified are not just restricted to plasma membrane but also includes novel transcription factors, miRNA and long noncoding RNA and list of genes that confer the programming needed to allow immune cells to extravasate into tumors.

The disclosed invention comprises lists of clinically relevant plasma membrane molecules that are overrepresented in all endothelial cells (Table 8) and specific segment of the vasculature such as venular endothelial cells (Table 9) and non-venular endothelial cells (Table 10) from murine and human tumors compared to their respective non-malignant tissues.

TABLE 8
A. Plasma membrane molecules over-represented in EC from tumor vs healthy shared by
human melanoma and pancreatic tumor (6 genes):
ENTPD1, MARCKS, SELP, APLNR, ROBO1, PLEKHO1
B. Plasma membrane molecules over-represented in EC from tumor vs healthy unique to
human melanoma (57 genes):
BTN3A2, SLCO2A1, SLC35G2, TNFSF10, PLIN2, ENG, PLVAP, PODXL, PPAP2A, RAMP3,
KDR, HLA-C, SLC6A6, INSR, TGFBR2, MLEC, HLA-DRA, VASP, C1QTNF5, EHD4, ITGA2,
HLA-DRB1, IFITM3, EFNA1, CALCRL, F2R, RELL1, VAMP5, CD40, SLC30A1, NRP1,
HLA-DOA, ESAM, THY1, BMPR2, ACVRL1, TM2B, MOB1A, SFRP1, SLC38A2, HEG1, CD99,
PPAP2B, SPRY4, ATP8B1, FZD6, ANXA5, CNIH1, DLL4, CSF2RB, CD164, TMEM165,
PLXND1, NT5E, RAB13, CD200, TMED10
C. Plasma membrane molecules over-represented in EC from tumor vs healthy unique to
human pancreatic tumor (23 genes):
CNKSR3, ASPH, STAB1, KCTD12, LEPR, PTP4A3, SVIL, ENPP2, TGFBR3, ITPR2, DSP,
FAP, BACE2, NRP2, CADM3, ACKR1, THSD7A, DST, CD93, SULF2, MCTP1, ADGRG6, TIE1
D. Plasma membrane molecules over-represented in EC from tumor vs healthy shared by
human pancreatic tumor, murine melanoma and/or murine colorectal cancer (9 genes):
VMP1, LAPTM5, EVL, PCDH17, ARRDC3, PMEPA1, MYOF, MMP14, PLEKHO1
E. Plasma membrane molecules over-represented in EC from tumor vs healthy shared by
human melanoma, murine melanoma and/or murine colorectal cancer (8 genes):
ACTR3, CD74, CLIC1, LAPTM4B, HLA-DQA1, TAPBP, MCAM, PLEKHO1
F. Plasma membrane molecules over-represented in EC from tumor vs healthy shared by
human melanoma, human pancreatic tumor, murine melanoma and murine colorectal cancer
(1 genes):
PLEKHO1

TABLE 9
A. Plasma membrane molecules over-represented in V-EC from tumor vs healthy shared by
human melanoma and pancreatic tumor (17 genes):
ENG, KDR, INSR, NRP1, ACVRL1, ROBO1, PKP4, CD200, ITGA2, CSF2RB, ENTPD1,
RELL1, TNFRSF1B, LAPTM4B, MARCKS, DYSF, PLXND1
B. Plasma membrane molecules over-represented in V-EC from tumor vs healthy unique to
human melanoma (50 genes):
SIGIRR, PTAFR, RAP1A, HLA-DMA, SYPL1, FAT4, HLA-DRB1, CERK, SYT15, CPD,
PTPRN2, HLADOA, THY1, FZD6, CNIH1, HLA-DQB2, IL3RA, BTN3A2, SLCO2A1,
TNFSF10, PLVAP, GPR68, CLSTN3, RAMP3, KL, HLA-C, HLA-DPB1, GBP5, DIAPH1,
HLA-DRA, EHD4, TMEM59, CX3CL1, ATP8A1, TSPAN7, LRRC8A, CD40, TACR1, IL10RB,
SLC30A1, SFRP1, BTN3A3, IL17RA, YIPF3, DLL1, ABI3, SEMA4C, SLC29A1, RAB13,
TMED10
C. Plasma membrane molecules over-represented in V-EC from tumor vs healthy unique to
human pancreatic tumor (63 genes):
TLR4, LEPR, ITGA5, ESYT1, RAC1, PAM, GNA14, ORAI2, ADGRL4, ASAP1, CADPS2,
TGFBR3, LRRC32, DSP, MET, LRP6, PPFIA1, OSBPL8, KRIT1, ANO2, PGRMC1, CLDN5,
EPS8, ADCY4, TMEM127, GRK5, IL13RA1, PLXDC2, NECTIN2, CADM3, PON2, ACKR1,
F2RL3, ITGA6, MFAP3, TIE1, CNKSR3, TJP1, FZD4, ENPP2, C1QTNF5, ITPR2, CALCRL,
EFNB2, CLEC1A, PNN, BACE2, ATP1B3, NRP2, FLT4, ITGA1, PPP4R3A, GPR146, CTTN,
CLTC, ATP2B4, ERLIN1, RIT1, USP9X, MCTP1, ADGRG6, ADGRF5, NCKAP1
D. Plasma membrane molecules over-represented in V-EC from tumor vs healthy shared by
human pancreatic tumor, murine melanoma and/or murine colorectal cancer (22 genes):
PCDH1, THSD7A, STAB1, PTPRG, PHACTR4, MLEC, GPR107, SEMA3F, CD93, EVL,
PCDH17, VMP1, BST2, MMP14, TM9SF2, ENTPD1, RELL1, TNFRSF1B, LAPTM4B,
MARCKS, DYSF, PLXND1
E. Plasma membrane molecules over-represented in V-EC from tumor vs healthy shared by
human melanoma, murine melanoma and/or murine colorectal cancer (23 genes):
TGFBR2, ESAM, IFNAR1, CD74, VAMP5, APLNR, HLA-DQA1, TMEM204, PCDH12,
MPZL1, F2R, GLG1, CLIC1, ACTR3, AMOTL1, PLEKHO1, MARCKS, DYSF, PLXND1,
ENTPD1, RELL1, TNFRSF1B, LAPTM4B, ACTR3, AMOTL1, PLEKHO1

TABLE 10
A. Plasma membrane molecules over-represented in NV-EC from tumor vs healthy shared by
human melanoma and pancreatic tumor (7 genes):
ENTPD1, MARCKS, APLNR, ROBO1, CD93, PCDH17, PLEKHO1
B. Plasma membrane molecules over-represented in NV-EC from tumor vs healthy unique to
human melanoma (71 genes):
APCDD1, JAG2, STX3, SLC35G2, HECW2, PLIN2, ENG, PLVAP, PODXL, RAMP3, MPZL2,
KDR, HLA-C, SLC6A6, INSR, TGFBR2, PLPP3, MLEC, HLA-DRA, VASP, JCAD, C1QTNF5,
ITGA2, MAGED2, HLA-DRB1, IFITM3, EFNA1, B4GALT1, CALCRL, F2R, VAMP5,
TSPAN12, LGALS9, PLPP1, TMEM30A, SLC30A1, GNB2, SELP, NRP1, FLT4, ESAM,
ABHD12, BMPR2, ACVRL1, ADGRL2, ITM2B, MOB1A, PKD2, SFRP1, SLC38A2, CDH5,
HEG1, CD99, CLIC4, GRK5, SPRY4, CLTC, ATP8B1, TAPBP, CNIH1, APP, DLL4, FLRT2,
DYSF, CD164, TMEM165, PLXND1, RAB13, GPR4, CD200, BSG
C. Plasma membrane molecules over-represented in NV-EC from tumor vs healthy unique to
human pancreatic tumor (30 genes):
STAB1, DLG1, KCTD12, LEPR, CACNA1C, PTP4A3, VMP1, TM4SF1, ATP11C, SVIL,
SLC4A7, ENPP2, DSP, PTPRE, NRP2, JAG1, EVL, PCSK5, SLC26A2, ACKR1, ATP2B1,
THSD7A, DST, ABCB1, FBLIM1, MCTP1, SULF2, ARRDC3, TIE1, KCNN3
D. Plasma membrane molecules over-represented in NV-EC from tumor vs healthy shared by
human pancreatic tumor, murine melanoma and/or murine colorectal cancer (6 genes):
FCGR2A, LAPTM5, PCDH17, PLEKHO1, PMEPA1, MMP14
E. Plasma membrane molecules over-represented in NV-EC from tumor vs healthy shared by
human melanoma, murine melanoma and/or murine colorectal cancer (13 genes):
SLCO2A1, VCAM1, GJA4, CD74, LAPTM4B, NT5E, TNFAIP1, EDNRB, ANXA5, PCDH17,
PLEKHO1, MCAM, CLIC1
F. Plasma membrane molecules over-represented in NV-EC from tumor vs healthy shared by
human melanoma, human pancreatic tumor, murine melanoma and murine colorectal cancer
(1 genes):
PLEKHO1

A more comprehensive list of genes including but not limited to transcription factors (TP), extracellular molecules, cytoplasmic molecules including miRNA. The tumor specific endothelial plasma membrane molecules will be use as targets for 1.) intra-tumoral specific gene delivery to venular endothelial cells to induce a venular programming (TF identified from the scRNA sequencing) that will increase intra-tumoral CD8 T cells and 2.) targeted delivery of therapeutics (CAR-T cells, TIL therapy, therapy comprising a cell expressing an antigen recognizing a tumor antigen, checkpoint blockade therapy and tumor specific chemotherapy delivery) with minimum off target effects. Molecules that are unique to human melanoma and/or human pancreatic tumors were identified. Many of these molecules are also upregulated in murine melanoma and/or colorectal adenocarcinoma tumor models, these molecules could be used for in vivo studies to characterize and demonstrate its tumor specificity with minimum off target effects and its ability to change the microvascular phenotype by increasing the “venuleness” of the venular segment of the vasculature. This will allow for increase immune cell recruitment in solid tumors. Molecules that are only over-represented in human tumors and not murine tumors will also be evaluated in other murine tumor models and other fresh human neoplastic tissues. Molecules that are in both human tumors could be also over-represented in other human solid tumors. The tables above list novel and unique plasma membrane molecules identified in human tumors. Other molecules identified are not just restricted to plasma membrane but also includes novel transcription factors, miRNA and long noncoding RNA and list of genes that confer the programming needed to allow immune cells to extravasate into tumors.

### Example 3: Characterize PMEPA1 Expression on Microvascular ECs of Murine and Human Solid Tumors

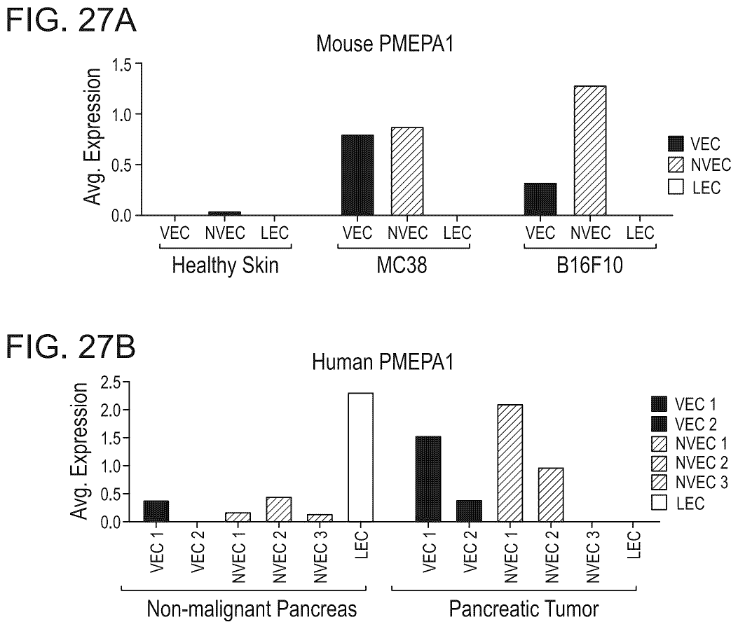

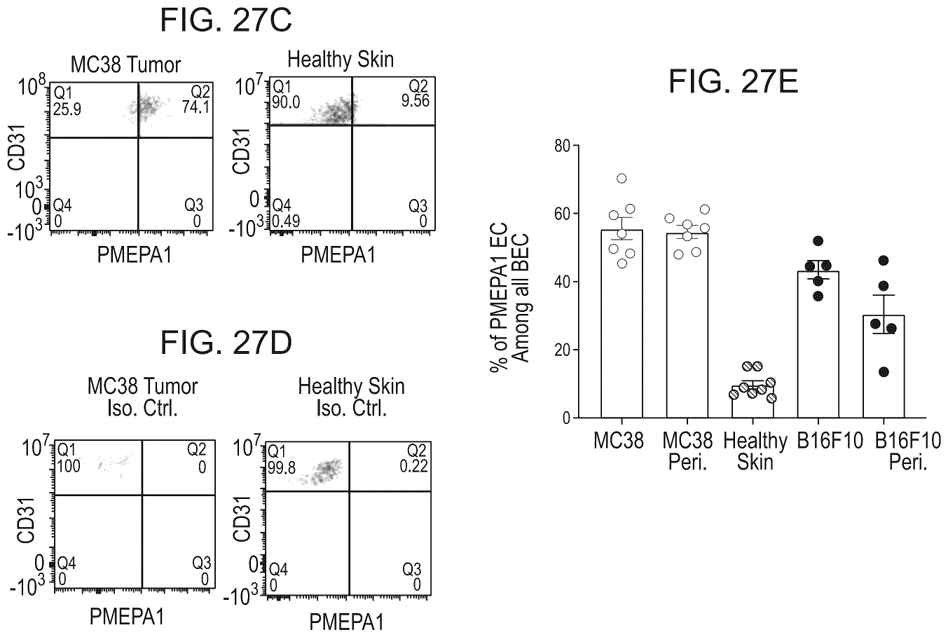

To determine PMEPA1 mRNA and protein expression and localization in primary human and murine tumors qPCR, FISH, flow cytometry and histology were be used. For murine tumor models, syngeneic colorectal adenocarcinoma (MC38) and melanoma (B16F10) tumor models were used, whereby tumor cells are implanted subcutaneously in the dorsal skin. MC38 is an immunogenic tumor with high T cell infiltrates (TILs) and responds to checkpoint blockade while B16F10 has the opposite characteristics. In FACS analysis of ECs from subcutaneously implanted syngeneic murine MC38 and B16F10 tumors, 74.1% of intratumoral ECs were PMEPA1+, whereas only 9.6% of ECs in normal skin expressed PMEPA1, and only at a very low level (FIGS. 27A-27E). For FACS analysis, the gating strategy for PMEPA1+ events was based on a polyclonal control antibody (Iso). The apparent basal level of PMEPA1 in healthy murine skin could reflect high background binding that is typical for many polyclonal Abs rather than true protein expression (FIG. 27A-27E). Indeed, healthy skin does not appear to express PMEPA1 at the mRNA level (FIG. 27A). PMEPA1 expression in fresh samples of human melanoma and peri-tumoral non-malignant skin, as well as, human pancreatic cancer and non-malignant pancreas at the protein level by flow cytometry and IHC were accessed.

### Example 4: Yeast Display sdAb Library to Generate and Validate sdAbs Against the PMEPA1 Ectodomain

Among these tumor EC restricted genes is Prostate Transmembrane Protein, Androgen Induced 1 (PMEPA1), a regulator of tissue responses to cytokines. PMEPA1 mRNA was significantly upregulated in ECs from MC38, B16F10 and human pancreatic cancer samples compared to ECs in nonmalignant peri-tumoral tissue (FIGS. 26A-26B and 27A-27E). PMEPA1 is also upregulated in human melanoma compared to healthy skin, however, it did not reach statistical significance in the analysis (red circles in FIG. 26A-26B). Additionally, PMEPA1 is present in 17 types of solid human tumors according to the TCGA database. Although EC expression of PMEPA1 in these tumors remains to be validated, it appears to be an attractive candidate to target ECs in a wide variety of tumors.

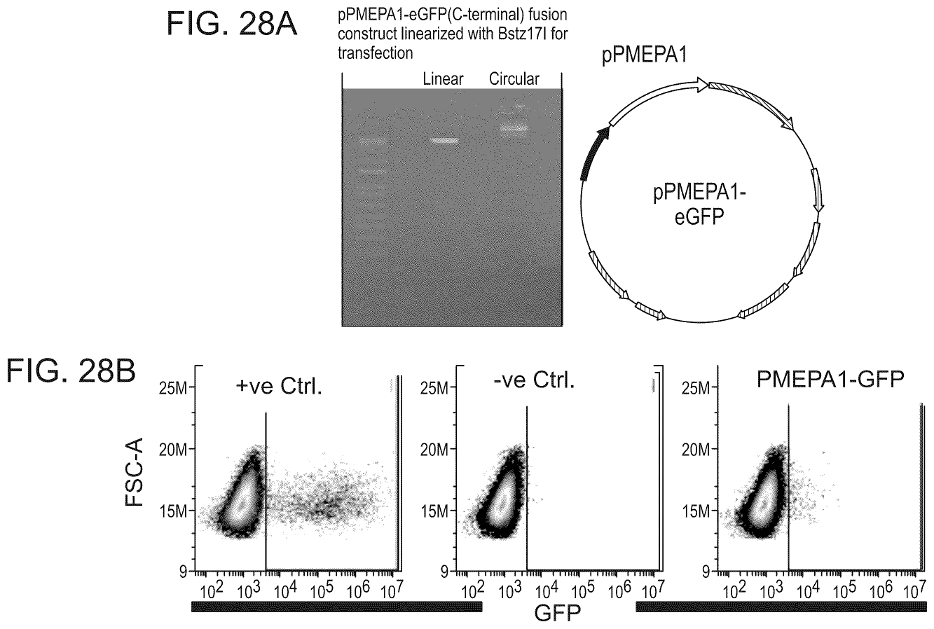

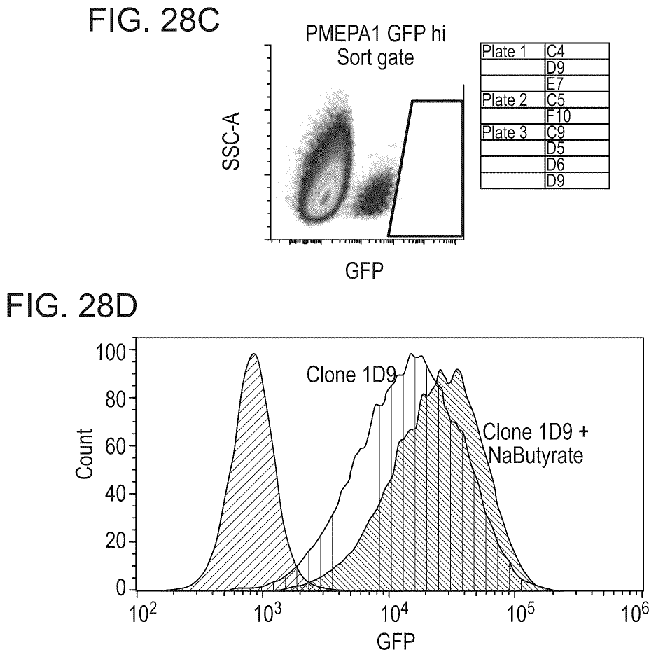

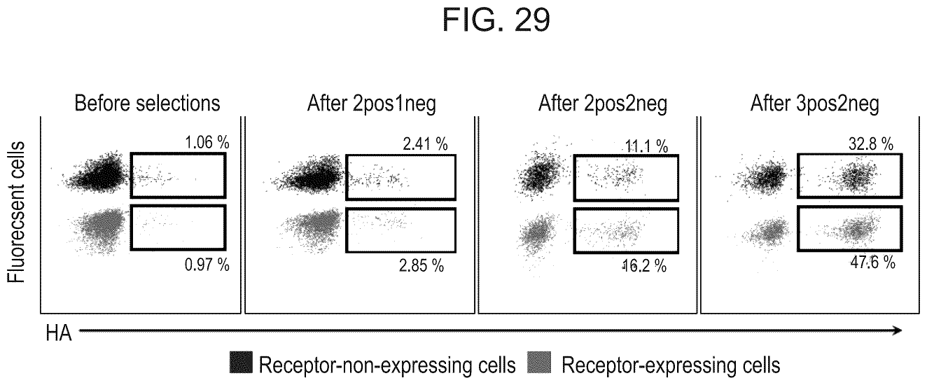

PMEPA1 mRNA and protein expression levels in normal and malignant human and murine tissues was validated. Mouse and human PMEPA1 have a single transmembrane domain and an ectodomain with 79% aa homology. While there are no reagents that specifically recognize this ectodomain, a commercially available polyclonal antibody to the cytoplasmic tail was used to validate at the protein level that PMEPA1 is preferentially expressed on tumor ECs (FIGS. 27A-27E). A yeast display library was used to raise sdAbs against human and murine PMEPA1 transfectants (FIGS. 28A-28D). Stably transfected L1.2 cells that express PMEPA1 fused with intracellular GFP were used. As negative and positive controls, L1.2 cells were transfected with empty vector or GFP alone (FIGS. 28A-28B). PMEPA1-GFP high cells were FACS sorted and subcloned by limiting dilution in 96-well plates and expanded in selection medium containing G418 (FIG. 28C). Clones which displayed consistently the highest mean fluorescence intensity were further expanded. Clone 1D9 that demonstrated the highest PMEPA1-GFP expression, which could be further enhanced by addition of sodium butyrate, an HDAC inhibitor, to the culture medium was selected (FIG. 28D). This clone is ready for use as ‘bait’ for the yeast display library to identify sdAb with reactivity against the ectodomain of PMEPA1. FIG. 29 shows an example where the yeast library was subjected alternatingly to three and two cycles of positive and negative selection, respectively. sdAb mediated yeast binding to target cells is readily detectable by FACS because sdAb expressing yeast cells coexpress a surface epitope from hemagglutinin (HA).

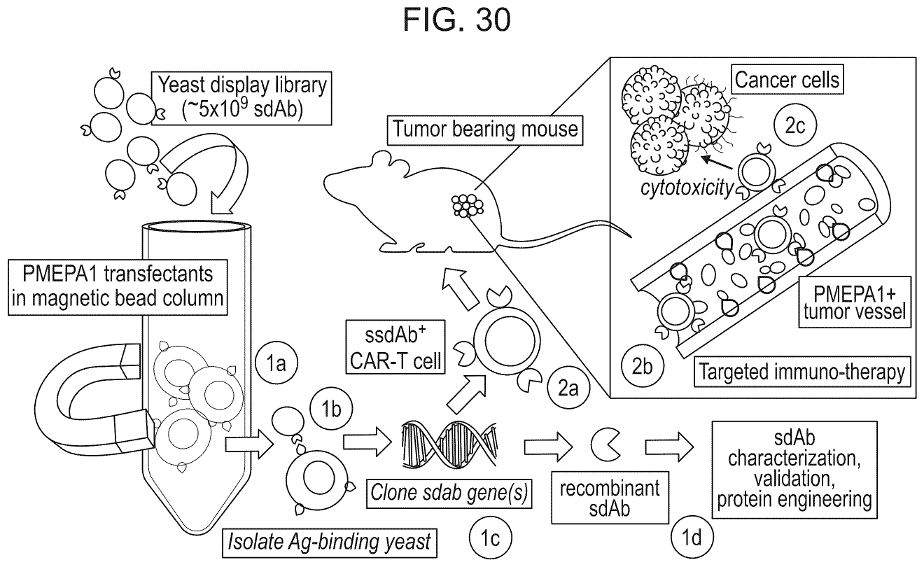

The yeast display library approach is based on performing a series of alternating magnetic-activated cell sorting (MACS)-based positive and negative selection steps followed by fluorescence-activating cell sorting (FACS)-based sorts (FIG. 30, Step 1a). For positive selections, PMEPA1-GFP expressing L1.2 cells will be labeled with anti-CD45 magnetic beads and loaded on magnetic columns. Next, sdAb-expressing yeast (which contains ˜5×10⁹ distinct sdAb clones) will be loaded on the same columns, and columns will be washed extensively. The contents of the columns will be retrieved, and yeast bound to L1.2 cells (determined as HA+ cells) will be sorted. Thus, the clones expressing relevant sdAb will be enriched. Each positive selection is followed by a negative selection cycle, whereby PMEPA1-negative L1.2 cells will be loaded on magnetic columns, followed by loading of sdAb-expressing yeast. The columns will be washed, and the unbound fraction will be collected, and HA+ yeast cells will be sorted. Yeast clones that bind to irrelevant surface antigens on L1.2 cells remain in the column and are eliminated (FIG. 30, Step 1b). Repeated cycles of positive and negative selection will result in decreased sdAb library diversity and increased efficiency of formation of cell-yeast conjugates, with enrichment for yeast clones that preferentially bind PMEPA1-expressing L1.2 cells. Upon reaching a high frequency of cell-yeast conjugates, the yeast sdAb library will be subcloned (FIG. 30, Step 1c), and the clones with maximum binding to PMEPA1-expressing L1.2 cells and no binding to control L1.2 cells will be identified. The sdAb encoding cDNAs will be subcloned into an expression vector and modified to append an N-terminal FLAG tag for protein purification and/or a C-terminal LPETG motif to allow for sortase A-mediated “click chemistry” linkage to acceptor moieties of interest. Recombinant sdAb will be expressed in E. coli and extensively characterized for reactivity with PMEPA1 in vitro (FACS, Western blot) and in situ using IHC and/or intravital microscopy of microvessels in tumors and non-malignant tissues at various anatomical location (FIG. 30, Step 1d). In addition, sdAbs will be engineered to allow surface expression/immobilization on CAR-T cells to test their ability to selectively target tumor ECs (FIG. 30, Steps 2a-2c).

### Example 5: Target CAR-T Cells with PMEPA1 sdAb to Tumor Microvessels and Assess Anti-Tumor Efficacy

Immobilized PMEPA1 sdAb on the surface of CAR T cells will be used to determine if these CAR T cells can be used as an effective targeted cell therapy. After IV infusion, the sdAb will enable CAR T cells to adhere selectively to tumor ECs that are normally non-adhesive for circulating T cells. Because of the immobilized PMEPA1 sdAb on the CAR T cells, it is expected that the CAR T cells will accumulate in solid tumors that are currently resistant to CAR T cell therapy. In particular, the lack of tumor targeting specificity of traditional CAR T cell therapies increases the risk for off-target effects and exhaustion. It is expected that sdAb-mediated targeting of such second-generation CAR T cells to tumors will further boost therapeutic efficacy.

The surface of the T cells that express a CAR specific for a tumor antigen will be decorated with PMEPA1 sdAb at a high density. In this setting, the sdAb will confer mechanical stability to CAR T cell binding to PMEPA1+ tumor microvessels, without transmitting an activating signal. As described above, at the transcriptional level, among all intravascular cells in mice only ECs lining the vasculature within tumors express robust levels of PMEPA1. Indeed, according to RNASeq data published by Immgen, among all murine cell types tested, the only healthy cells that express PMEPA1 are brain microglia (and, to a lesser degree, alveolar macrophages) (www.rstats.immgen.org/Skyline/skyline.html). After adoptive transfer, CAR T cell are unlikely to access the CNS because normal brain ECs do not express PMEPA1 and do not support substantial T cell trafficking. Therefore, sdAb decoration should focus CAR T cells onto tumor ECs without redirecting them to other cell types or anatomic sites. In humans, according to the human cell atlas website (www.humancellatlas.org), only a subset of PBMCs (Siglec6-CD123+CD11c-PBMC) from healthy patients had mRNA levels slightly above baseline. Detection of PMEPA1 has been reported only at the mRNA level in these databases, which does not always correlate with the presence of protein, especially if the RNA level is low. For the most rigorous analysis, a reliable antibody detecting the ectodomain of this transmembrane molecule is needed to allow for sensitive assessment by flow cytometry and immuno-histochemistry. Due to the absence of such a reagent, very few studies have been performed on this molecule to date.

Although it is expected that the sdAb-targeted CAR T cells will be highly selective for tumor ECs, there may be other PMEPA1+ target cells. However, even if this were the case, expression of PMEPA1 sdAb without a signaling domain should not cause increased toxicity as long as such hypothetical PMEPA1+ cells do not also express the CAR antigen or other means to activate CAR T cells.

A schematic of the proposed protocol is shown in FIG. 30. Briefly, CAR-T cells express chimeric Ag receptors (CARs) that link an extracellular Ag recognition component to an intracellular signaling domain resulting in T cell activation when a tumor Ag is encountered. CAR T cells will be generated against human CD20. These CAR T cells will be modified to display one or more PMEPA1 sdAbs on their surface by transfecting CAR T cells with chimeric sdAbs containing either a cytoplasmic and transmembrane domain and a linker region or a Gpi anchor (FIG. 30, Step 2a). The PMEPA1 sdAb constructs will not include an activating signaling domain. The sdAb may function as an anchor to immobilize CAR T cells within tumor microvessels upon adoptive transfer into tumor bearing mice. Thus, adoptive transfer experiments and in situ imaging in tumor bearing mice will be performed to determine whether surface displayed sdAbs enhance CAR T cell accumulation in tumors (FIG. 30, Step 2b). MC38 or B16F10 solid tumors will be transduced to express human CD20 with anti-human CD20 CAR T cells that will be surface modified either with anti-PMEPA1 sdAbs or a non-binding control sdAb. Assessment whether CAR T cell decoration with anti-PMEPA1 sdAb confers selective targeting of CAR T cells to tumors and enhancement of anti-tumor immunity will be performed (i.e. suppression of tumor growth, survival etc.) (FIG. 30, Step 2c).

The anti-PMEPA1 sdAb serving as the Ag binding domain of a CAR will also be tested to determine if T cell activation after recognition of PMEPA1 by the sdAb-CAR results in tumor EC killing. In this setting, the CAR T cells would exert cytotoxic activity towards tumor ECs, destroying the tumor by going after these vital stromal cells rather than the tumor cells themselves. While this approach would likely result in rapid killing of host tumors since CAR T cells will initially be present at a high density in the blood stream, there may be a greater risk for on-target off-tumor side effects due to recognition of PMEPA1 on cells other than intra-tumoral ECs. Thus, recipient animals' health and potential organ damage will be monitored. If off-target toxicity is unacceptable, it could offer a powerful new treatment modality for solid tumors because the CAR T cells could function within the tumor vessel lumen, without the need to extravasate.

### REFERENCES

(1) Folkman, J. Tumor angiogenesis: therapeutic implications. N Engl J Med, 285, 1182-1186, (1971).
(2) Peske J D, Woods A B, Engelhard V H. Control of CD8 T-Cell Infiltration into Tumors by Vasculature and Microenvironment. Adv Cancer Res., 128, 263-307, (2015).
(3) Potente, M., Mäkinen, T. Vascular heterogeneity and specialization in development and disease. Nat Rev Mol Cell Biol 18, 477-494 (2017).
(4) Halin, C., J. Rodrigo Mora, C. Sumen, and U. H. von Andrian. In vivo imaging of lymphocyte trafficking. Annu Rev Cell Dev Biol, 21, 581-603, (2005).
(5) Ley, K., and P. Gaehtgens. Endothelial, not hemodynamic, differences are responsible for preferential leukocyte rolling in rat mesenteric venules. Circ Res, 69, 1034-1041, (1991).
(6) Lawson, N. D., and B. M. Weinstein. Arteries and veins: making a difference with zebrafish. Nat Rev Genet, 3, 674-682, (2002).
(7) von Andrian, U. H., and C. R. Mackay. 2000. T-cell function and migration. Two sides of the same coin. N. Engl. J. Med., 343, 1020-1034, (2000).
(8) Rocha, S. F., and R. H. Adams. Molecular differentiation and specialization of vascular beds. Angiogenesis, 12, 139-147, (2009).
(9) Oliver, G., and R. S. Srinivasan. Endothelial cell plasticity: how to become and remain a lymphatic endothelial cell. Development, 137, 363-372, (2010).
(10) Thiriot, A. et al. Differential DARC/ACKR1 expression distinguishes venular from non-venular endothelial cells in murine tissues. BMC Biology, 15, 45, (2017).
(11) Ngiow, S. F. et al. A Threshold Level of Intratumor CD8+ T-cell PD1 Expression Dictates Therapeutic Response to Anti-PD1. Cancer Research, 75, 3800-3811, (2015).
(12) Vikram R. Juneja, Kathleen A. McGuire, Robert T. Manguso, Martin W. LaFleur, Natalie Collins, W. Nicholas Haining, Gordon J. Freeman, Arlene H. Sharpe. PD-L1 on tumor cells is sufficient for immune evasion in immunogenic tumors and inhibits CD8 T cell cytotoxicity. J Exp Med, 214, 895-904, (2014).
(13) Weninger, W., M. A. Crowley, N. Manjunath, and U. H. von Andrian. Migratory properties of naive, effector, and memory CD8 (+) T cells. J. Exp. Med. 194, 953-966, (2001).
(14) Gerlach, C. et. al. The Chemokine Receptor CX3CR1 Defines Three Antigen-Experienced CD8 T Cell Subsets with Distinct Roles in Immune Surveillance and Homeostasis. Immunity, 45, 1270-1284, (2016).
(15) Gierahn, T. M. et al. Seq-Well: portable, low-cost RNA sequencing of single cells at high throughput. Nature Methods 14, 395-398, (2017).
(16) Wolf, K. et. al. Molecular identity of arteries, veins, and lymphatics. J. Vasc. Surgery., 69, 253-262, (2019).
(17) Nathaniel G. de la Paz and Patricia A. D'Amore. Arterial versus venous endothelial cells. Cell tissue, 335, 5-16, (2014).
(18) Zhao, Q. et. al. Single-Cell Transcriptome Analyses Reveal Endothelial Cell Heterogeneity in Tumors and Changes following Antiangiogenic Treatment. Cancer Research, 78, 2370-2383, (2018).
(19) Iso, T. et. al. Capillary Endothelial Fatty Acid Binding Proteins 4 and 5 Play a Critical Role in Fatty Acid Uptake in Heart and Skeletal Muscle. Arteriosclerosis, Thrombosis, and Vascular Biology, 33, 2549-2557, (2013).
(20) Rousseeuw, P. J. Silhouettes: A graphical aid to the interpretation and validation of cluster analysis. Journal of Computational and Applied Mathematics, 20, 53-65, (1987).
(21) Dong, X. et. al. Crystal Structure of the V Domain of Human Nectin-like Molecule-1/Syncam3/Tsll1/Igsf4b, a Neural Tissue-specific Immunoglobulin-like Cell-Cell Adhesion Molecule. JBC, 281, 10610-10617, (2006).
(22) Wang X, Abraham S, Mckenzie J A, Jeffs N, Swire M, Tripathi V B, Luhmann U F, Lange C A, Zhai Z, Arthur H M, Bainbridge J W, Moss S E, Greenwood J. LRG1 promotes angiogenesis by modulating endothelial TGF-β signalling. Nature, 499, 306-11, (2013).
(23) Hanzelmann, S., Castelo, R. & Guinney, J. GSVA: gene set variation analysis for microarray and RNA-Seq data. BMC Bioinformatics, 14, 7, (2013).
(24) Kalucka, J. et. al. Single-Cell Transcriptome Atlas of Murine Endothelial Cells. Cell, 180, 764-779, (2020).
(25) Muz, B., de la Puente, P., Azab, F., & Azab, A. K. The role of hypoxia in cancer progression, angiogenesis, metastasis, and resistance to therapy. Hypoxia (Auckland, N.Z.), 3, 83-92, (2015).
(26) Tran-Dinh et. al., HDL and endothelial protection. BJP, 169, 493-511, (2013).
(27) Narimasa Miho, Takafumi Ishida, Noriko Kuwaba, Mari Ishida, Keiko Shimote-Abe, Kumiko Tabuchi, Tetsuya Oshima, Masao Yoshizumi, Kazuaki Chayama. Role of the JNK pathway in thrombin-induced ICAM-1 expression in endothelial cells. Cardiovascular Research, 68, 289-298, (2005).
(28) Saul, L., Ilieva, K. M., Bax, H. J., Karagiannis, P., Correa, I., Rodriguez-Hernandez, I., Josephs, D. H., Tosi, I., Egbuniwe, I. U., Lombardi, S., Crescioli, S., Hobbs, C., Villanova, F., Cheung, A., Geh, J. L., Healy, C., Harries, M., Sanz-Moreno, V., Fear, D. J., Spicer, J. F., . . . Karagiannis, S. N. IgG subclass switching and clonal expansion in cutaneous melanoma and normal skin. Scientific reports, 6, 29736, (2016).
(29) Prabir Kumar Chakraborty and Soumyajit Banerjee Mustafi. Heat Shock Proteins: Heatingup skin cancer biology. Journal of Dermatitis, 1, 1-4, (2015).
(30) Muchemwa, Faith Chengetayi; Nakatsura, Tetsuya; Fukushima, Satoshi; Nishimura, Yasuharu; Kageshita, Toshiro; Ihn, Hironobu. Differential expression of heat shock protein 105 in melanoma and melanocytic naevi. Melanoma Research, 18, 166-171, (2008).
(31) Pleunie van den Borne, Paul H. A. Quax, Imo E. Hoefer, and Gerard Pasterkamp. The Multifaceted Functions of CXCL10 in Cardiovascular Disease. Biomed Research International, 2014, 893106, (2014).
(32) Gabriele S. V. Campanella, Jan Grimm, Lindsay A. Manice, Richard A. Colvin, Benjamin D. Medoff, Gregory R. Wojtkiewicz, Ralph Weissleder, Andrew D. Luster. Oligomerization of CXCL10 Is Necessary for Endothelial Cell Presentation and In vivo Activity. The Journal of Immunology, 177, 6991-6998, (2006).
(33) Ji, P., Diederichs, S., Wang, W. et al. MALAT-1, a novel noncoding RNA, and thymosin B4 predict metastasis and survival in early-stage non-small cell lung cancer. Oncogene 22, 8031-8041 (2003).
(34) Xue, Aiqun MSc*: Scarlett, Christopher J. PhD*: Jackson, Christopher J. PhDt: Allen, Barry J. DSc, FAPS: Smith, Ross C. MD, FRACS* Prognostic Significance of Growth Factors and the Urokinase-Type Plasminogen Activator System in Pancreatic Ductal Adenocarcinoma. Pancreas, 36, 160-167, (2008).
(35) Chen, E. Y., Tan, C. M., Kou, Y. et al. Enrichr: interactive and collaborative HTML5 gene list enrichment analysis tool. BMC Bioinformatics 14, 128 (2013).
(36) Kuleshov, M. V., Jones, M. R., Rouillard, A. D., Fernandez, N. F., Duan, Q., Wang, Z., Koplev, S., Jenkins, S. L., Jagodnik, K. M., Lachmann, A., McDermott, M. G., Monteiro, C. D., Gundersen, G. W., & Ma'ayan, A. Enrichr: a comprehensive gene set enrichment analysis web server 2016 update. Nucleic acids research, 44 (W1), W90-W97, (2016).
(37) Dunham, I., Kundaje, A., Aldred, S. et al. An integrated encyclopedia of DNA elements in the human genome. Nature 489, 57-74 (2012).
(38) Davis, C. A., Hitz, B. C., Sloan, C. A., Chan, E. T., Davidson, J. M., Gabdank, I., Hilton, J. A., Jain, K., Baymuradov, U. K., Narayanan, A. K., Onate, K. C., Graham, K., Miyasato, S. R., Dreszer, T. R., Strattan, J. S., Jolanki, O., Tanaka, F. Y., & Cherry, J. M. The Encyclopedia of DNA elements (ENCODE): data portal update. Nucleic acids research, 46 (D1), D794-D801, (2018).
(39) Mogensen Trine H. IRF and STAT Transcription Factors—From Basic Biology to Roles in Infection, Protective Immunity, and Primary Immunodeficiencies. Frontiers in Immunology, 9, 3047, (2019).
(40) Min, Y., Ghose, S., Boelte, K. et al. C/EBP-δ regulates VEGF-C autocrine signaling in lymphangiogenesis and metastasis of lung cancer through HIF-1α. Oncogene 30, 4901-4909 (2011).
(41) Wen, Y., Zhou, X., Lu, M. et al. Bclaf1 promotes angiogenesis by regulating HIF-1α transcription in hepatocellular carcinoma. Oncogene 38, 1845-1859 (2019).
(42) Song, H., Suehiro, J., Kanki, Y., Kawai, Y., Inoue, K., Daida, H., Yano, K., Ohhashi, T., Oettgen, P., Aird, W. C., Kodama, T., & Minami, T. Critical role for GATA3 in mediating Tie2 expression and function in large vessel endothelial cells. The Journal of biological chemistry, 284, 29109-29124, (2009).
(43) Fu, M. et. al. Egr-1 target genes in human endothelial cells identified by microarray analysis. Gene, 315, 33-41, (2003).
(44) Vicari, A. P. et. al. TECK: A Novel CC Chemokine Specifically Expressed by Thymic Dendritic Cells and Potentially Involved in T Cell Development. Immunnity, 7, 291-301, (1997).
(45) Luster, A. D. et. al. Molecular and biochemical characterization of a novel y-interferon-inducible protein. JBC, 263, 12036-12043, (1988).
(46) Zhang, H. et. al. mRNA-Binding Protein ZFP36 Is Expressed in Atherosclerotic Lesions and Reduces Inflammation in Aortic Endothelial Cells. Arteriosclerosis, Thrombosis, and Vascular Biology, 33, 1212-1220, (2013).
(47) Cohnheim J. Lectures on General Pathology: A Handbook for Practitioners and Students. London: The New Sydenham Society; 1889.
(48) Yano K, Gale D, Massberg S, Cheruvu P K, Monahan-Earley R, Morgan E S, Haig D, von Andrian U H, Dvorak A M, Aird W C. Phenotypic heterogeneity is an evolutionarily conserved feature of the endothelium. Blood, 109, 613-5, (2007).
(49) Atherton A, Born G V R. Quantitative investigation of the adhesiveness of circulating polymorphonuclear leucocytes to blood vessel walls. J Physiol., 222, 447-74, (1972).
(50) von Andrian U H, Chambers J D, McEvoy L M, Bargatze R F, Arfors K E, Butcher E C. Two-step model of leukocyte-endothelial cell interaction in inflammation: distinct roles for LECAM-1 and the leukocyte beta 2 integrins in vivo. Proc Natl Acad Sci USA, 88, 7538-42, (1991).
(51) T. M. Gierahn, M. H. 2. Wadsworth, T. K. Hughes, B. D. Bryson, A. Butler, R. Satija, S. Fortune, J. C. Love and A. K. Shalek. Seq-Well: portable, low-cost RNA sequencing of single cells at high throughput,” Nat Methods, pp. 395-398, 13 Apr. 2017.
(52) A. Dobin and e. al., “STAR: ultrafast universal RNA-seq aligner,” Bioinformatics, no. 29, pp. 15-21, 2013.
(53) E. Z. Macosko, “Highly parallel genome-wide expression profiling of individual cells using nanoliter droplets.,” Cell, no. 161, pp. 1202-1214, 2015.
(54) R. Satija, J. A. Farrell, D. Gennert, A. F. Schier and A. Regev, “Spatial reconstruction of single-cell gene expression data,” Nat. Biotechnol., no. 33, pp. 495-502, 2015.
(55) F. A. Wolf, P. Angerer and F. J. Theis, “SCANPY: large-scale single-cell gene expression data analysis.,” Genome Biol., no. 19, p. 15, 2018.
(56) E. e. a. Becht, “Dimensionality reduction for visualizing single-cell data using UMAP.,” Nat. Biotechnol., no. 37, pp. 38-44, 2018.
(57) C. S. McGinnis, L. M. Murrow and Z. J. Gartner, “DoubletFinder: Doublet Detection in Single-Cell RNA Sequencing Data Using Artificial Nearest Neighbors,” Cell Systems, vol. 8, no. 4, pp. 329-337, 2019.
(58) Stuart, T., Butler, A. et. al. Comprehensive Integration of Single-Cell Data. Cell, 177, 1888-1902, (2019).
(59) Rousseeuw, P. J. Silhouettes: A graphical aid to the interpretation and validation of cluster analysis. Journal of Computational and Applied Mathematics, 20, 53-65, (1987).
(60) Hänzelmann, S., Castelo, R. & Guinney, J. GSVA: gene set variation analysis for microarray and RNA-Seq data. BMC Bioinformatics, 14, 7, (2013).
(61) Kalucka, J. et. al. Single-Cell Transcriptome Atlas of Murine Endothelial Cells. Cell, 180, 764-779, (2020).
(62) Smyth G K. Linear models and empirical bayes methods for assessing differential expression in microarray experiments. Stat Appl Genet Mol Biol. 3, Article3, (2004).
(63) Chen, E. Y., Tan, C. M., Kou, Y. et al. Enrichr: interactive and collaborative HTML5 gene list enrichment analysis tool. BMC Bioinformatics 14, 128 (2013).
(64) Kuleshov, M. V., Jones, M. R., Rouillard, A. D., Fernandez, N. F., Duan, Q., Wang, Z., Koplev, S., Jenkins, S. L., Jagodnik, K. M., Lachmann, A., McDermott, M. G., Monteiro, C. D., Gundersen, G. W., & Ma'ayan, A. Enrichr: a comprehensive gene set enrichment analysis web server 2016 update. Nucleic acids research, 44 (W1), W90-W97, (2016).

### INCORPORATION BY REFERENCE

The entire disclosure of each of the patent documents, including patent application documents, scientific articles, governmental reports, websites, and other references referred to herein is incorporated by reference herein in its entirety for all purposes. In case of a conflict in terminology, the present specification controls. All sequence listings, or Seq. ID. Numbers, disclosed herein are incorporated herein in their entirety.

The following references, to the extent that they provide exemplary procedural or other details supplementary to those set forth herein, are specifically incorporated herein by reference.

Although illustrative embodiments of the present invention have been described herein, it should be understood that the invention is not limited to those described, and that various other changes or modifications may be made by one skilled in the art without departing from the scope or spirit of the invention.

1. A targeting molecule, wherein the targeting molecule binds to a transmembrane molecule on a tumor vascular endothelial cell in which expression of the transmembrane molecule is upregulated and wherein the transmembrane molecule is selected from the group consisting of molecules set forth in Tables 8-10.

2. The targeting molecule of claim 1, wherein the tumor vascular endothelial cell is a venular cell.

3. The targeting molecule of claim 1, wherein:
(a) the transmembrane molecule is not expressed in non-tumor vascular endothelial cells, the transmembrane molecule is expressed at higher levels in the tumor vascular endothelial cells as compared to in non-tumor vascular endothelial cells, or the transmembrane molecule is a variant of a transmembrane protein expressed in non-tumor vascular endothelial cells, optionally, wherein the transmembrane molecule is expressed at least 1.5-fold, at least 2-fold, at least 2.5-fold, at least 3-fold, at least 3.5-fold, at least 4-fold, at least 4.5-fold, or at least 5-fold more in the tumor vascular endothelial cells as compared to expression in non-tumor vascular endothelial cells; and/or
(b) the expression of the transmembrane molecule is upregulated as compared to a control level, optionally, wherein the control level is the level of expression of the transmembrane molecule in a non-tumor vascular endothelial cell; and/or
(c) the targeting molecule is an antibody or antigen-binding fragment thereof, optionally, wherein the antibody or antigen-binding fragment is a monoclonal antibody, human antibody, a humanized antibody, a chimeric antibody, a recombinant antibody, a multispecific antibody, or an antigen-binding fragment thereof; optionally, wherein the antigen-binding fragment is 1) an Fv, Fab, F(ab′)2, Fab′, dsFv, scFv, or sc(Fv)2; 2) a diabody, ScFv, SMIP, single chain antibody, affibody, avimer, or nanobody; or 3) a single domain antibody.

4.-9. (canceled)

10. A composition comprising 1) the targeting molecule of claim 1, and 2) an agent that (a) induces cell death to a tumor cell in which the expression of at least one transmembrane molecule selected from the group consisting of those molecules set forth in Tables 8-10 is upregulated as compared to a non-tumor vascular endothelial control cell, optionally wherein the tumor cell is a tumor vascular cell or tumor stromal cell, or (b) induces an inflammatory response.

11. The composition of claim 10, wherein:
(a) the agent that induces cell death is an agent that induces immunogenic cell death; or
(b) the agent that induces cell death is an agent that induces non-immunogenic cell death; or
(c) the agent is selected from the group consisting of a small molecule, saccharide, oligosaccharide, polysaccharide, peptide, protein, peptide analog and derivatives, peptidomimetic, siRNAs, shRNAs, antisense RNAs, ribozymes, dendrimers, aptamers, and any combination thereof; or
(d) the agent that induces an inflammatory response is a TLR4 agonist or GP-130 agonist; or
(e) the agent that induces cell death is a chemotherapeutic agent; or
(f) the agent that induces cell death is an engineered CAR-immune cell, optionally the CAR-immune cell is a CAR-T cell, CAR-macrophages, CAR-monocyte, CAR-granulocyte, CAR-NK cell, a CAR-NKT cell, a tumor infiltrating lymphocyte (TIL), a cell expressing an antigen recognizing a tumor antigen or a cell expressing a receptor recognizing an antibody bound to the surface of a tumor cell; and/or
(g) the agent is coupled to or is co-administered with the targeting molecule.

12.-17. (canceled)

18. A pharmaceutical composition comprising 1) the targeting molecule of claim 1, and 2) a pharmaceutically acceptable carrier.

19. The pharmaceutical composition of claim 18, wherein the pharmaceutical composition comprises a lipid formulation, optionally, wherein the lipid formulation comprises a lipid nanoparticle.

20. (canceled)

21. (canceled)

22. A method of treating cancer in a subject in need thereof,
wherein the cancer is characterized by a tumor vascular endothelial cell in which the expression of at least one transmembrane molecule is upregulated, comprising administering to the subject a composition comprising a targeting molecule which binds to the transmembrane molecule on the tumor vascular endothelial cell and an agent that induces cancer cell death, optionally wherein the composition is a composition of claim 10.

23. A method of treating cancer in a subject in need thereof, comprising administering to the subject the composition of claim 10.

24. The method of claim 23, wherein
(a) the agent is coupled to the targeting molecule;
(b) the agent is co-administered with the targeting molecule;
(c) the agent is co-administered with a lipid nanoparticle comprising the targeting molecule; and/or
(d) wherein the expression of the transmembrane molecule is upregulated as compared to a control level, optionally wherein the control level is the level of expression of the transmembrane molecule in a non-tumor vascular endothelial cell.

25.-27. (canceled)

28. The method of claim 22,
(a) further comprising identifying in the subject the presence of the tumor vascular endothelial cell in which the expression of the at least one transmembrane molecule is upregulated as compared to expression of the transmembrane molecule in a non-tumor vascular endothelial cell and wherein the transmembrane molecule is selected from the group consisting of molecules set forth in Tables 8-10; and/or
(b) wherein the method elicits or enhances an immune response to the cancer, wherein the method increases the level or activity of intra-tumoral T cells, optionally wherein the level or activity of intra-tumoral T cells are increased at least 1.5-fold, at least 2-fold, at least 2.5-fold, at least 3-fold, at least 3.5-fold, at least 4-fold, at least 4.5-fold, or at least 5-fold more after administration as compared to the level or activity of intra-tumoral T cells prior to administration.

29.-31. (canceled)

32. A method of treating cancer in a subject in need thereof, comprising modifying the expression in a tumor vascular endothelial cell of at least one transmembrane molecule which is upregulated, optionally wherein the transmembrane molecule is selected from the group consisting of those molecules set forth in Tables 8-10, wherein modifying the expression of at least one transmembrane molecule comprises delivering a nucleic acid capable of modifying gene expression of the at least one transmembrane molecule.

33. The method of claim 32,
(a) further comprising identifying in the subject the presence of the tumor vascular endothelial cell in which the expression of the at least one transmembrane molecule is upregulated as compared to a control level, optionally wherein the control level is the level of expression of the transmembrane molecule in a non-tumor vascular endothelial control cell;
(b) wherein the nucleic acid is selected from the group consisting of an antisense RNA, siRNA, shRNA, and a CRISPR system, optionally
(1) wherein the antisense RNA, siRNA, or shRNA targets an mRNA of at least one transmembrane molecule; or
(2) wherein the CRISPR system comprises i) one or more guide RNAs (gRNAs), wherein the gRNA targets at least one transmembrane molecule gene or promoter region; and ii) a Cas9 protein, wherein the Cas9 protein is nuclease deficient (dCas9),
(i) wherein the dCas9 protein further comprises an effector molecule, wherein the effector molecule is selected from the group consisting of DNA-binding domain, epigenetic modifier, and a nuclease,
wherein the DNA-binding domain is a DNA-binding domain from a Transcription activator-like effector (TALE) polypeptide or a zinc finger (ZNF) polypeptide; and/or (ii) wherein the epigenetic modifier is selected from the group consisting of a DNA methyltransferase, histone acetyltransferase, histone deacetylase, histone methyltransferase, and histone demethylase.

34.-42. (canceled)

43. The method of claim 32:
(a) wherein the nucleic acid is present in a viral expression vector, optionally, wherein the viral expression vector is present in a pharmaceutical composition comprising a lipid formulation comprising a targeting molecule, optionally, wherein the targeting molecule binds to the transmembrane molecule on a tumor vascular endothelial cell in which expression of the transmembrane molecule is upregulated and wherein the transmembrane molecule is selected from the group consisting of molecules set forth in Tables 8-10;
(b) wherein the targeting molecule binds the same transmembrane molecule that the nucleic acid is capable of modifying expression of, or wherein the targeting molecule binds a different transmembrane molecule than the nucleic acid is capable of modifying expression of; and/or
(c) wherein the at least one transmembrane molecule is not expressed in non-tumor vascular endothelial cells, the at least one transmembrane molecule is expressed at higher levels in the tumor vascular endothelial cells as compared to in non-tumor vascular endothelial cells, or the transmembrane molecule is a variant of a transmembrane protein expressed in non-tumor vascular endothelial cell; and/or
(d) further comprising
(1) determining that expression of the at least one transmembrane molecule has been decreased as compared to a control cell that has not been administered the nucleic acid; or
(2) determining that expression of the at least one transmembrane molecule has been increased as compared to a control cell that has not been administered the nucleic acid.

44.-52. (canceled)

53. A method of treating cancer in a subject in need thereof, comprising:
1) administering to a subject having cancer an immune effector cell expressing a chimeric antigen receptor (CAR), wherein the CAR comprises a targeting molecule of claim 1, wherein the targeting molecule binds to a transmembrane molecule on a tumor vascular endothelial cell in which expression of the transmembrane molecule is upregulated; or
2) administering to a subject having cancer an immune effector cell expressing a chimeric antigen receptor (CAR), wherein a targeting molecule of claim 1 is expressed on the cell surface of the immune effector cell, wherein the targeting molecule binds to a transmembrane molecule on a tumor vascular endothelial cell in which expression of the transmembrane molecule is upregulated.

54. (canceled)

55. The method of claim 53:
(1) wherein the immune effector cell is a T cell, macrophage, monocyte, granulocyte, natural killer (NK) cell, or natural killer T (NKT);
(2) further comprising identifying in the subject the presence of the tumor vascular endothelial cell in which the expression of the at least one molecule is upregulated as compared to in a non-tumor vascular endothelial cell;
(3) wherein the method elicits or enhances an immune response to the cancer, optionally by increasing the level or activity of intra-tumoral T cells, wherein the level or activity of intra-tumoral T cells is increased at least 1.5-fold, at least 2-fold, at least 2.5-fold, at least 3-fold, at least 3.5-fold, at least 4-fold, at least 4.5-fold, or at least 5-fold more after administration as compared to the level or activity of intratumoral T cells to prior administration; and/or
(4) wherein the method elicits an inflammatory response.

56.-59. (canceled)

60. A method of diagnosing or prognosing cancer in a subject, comprising determining the expression of at least one transmembrane molecule selected from the group consisting of those molecules set forth in Tables 8-10 on a tumor vascular endothelial cell, wherein upregulation of expression of the at least one molecule on the tumor vascular endothelial cell as compared to a control level is indicative of the presence or progression of the cancer, optionally wherein the control level is the level of expression of the transmembrane molecule in a non-tumor vascular endothelial cell.

61. (canceled)

62. A method of determining the efficacy of treatment of cancer in a subject, comprising
i) determining the expression of at least one transmembrane molecule selected from the group consisting of those molecules set forth in Tables 8-10 on a tumor vascular endothelial cell prior to administering a cancer treatment, wherein increased expression of the at least one transmembrane molecule on the tumor vascular endothelial cell as compared to a control level is indicative of the presence or progression of the cancer;
ii) determining the expression of the at least one transmembrane molecule after administration of the cancer treatment, wherein decreased expression of the at least one transmembrane molecule as compared to a control level is indicative of effective cancer treatment,
optionally, wherein the control level is the expression of the transmembrane molecule on a tumor vascular endothelial cell prior to administering the cancer treatment.

63. (canceled)

64. A composition comprising the targeting molecule of claim 1 associated with a detectable marker,
optionally, wherein the detectable marker is selected from the group consisting of fluorescent labels, phosphorescent labels, chemiluminescent labels or bioluminescent labels, radio-isotopes, metals, metals chelates or metallic cations, chromophores and enzymes.

65. (canceled)

66. A medical imaging method comprising (i) administering the composition of claim 64, and (ii) detecting the targeting molecule in the body of the patient.

67. The method of claim 22, wherein:
(a) the tumor vascular endothelial cell is a venular cell;
(b) the transmembrane molecule is not expressed in non-tumor vascular endothelial cells, the molecule is expressed at higher levels in the tumor vascular endothelial cells as compared to non-tumor vascular endothelial cells, or the transmembrane molecule is a variant of a transmembrane protein expressed in non-tumor vascular endothelial cell, and/or
(c) the transmembrane molecule is expressed at least 1.5-fold, at least 2-fold, at least 2.5-fold, at least 3-fold, at least 3.5-fold, at least 4-fold, at least 4.5-fold, or at least 5-fold more in tumor vascular endothelial cells as compared to in non-tumor vascular endothelial cells.

68.-69. (canceled)

70. The method of claim 22, wherein the cancer is
(i) a non-immunogenic cancer;
(ii) a hematological cancer;
(iii) a solid tumor;
(iv) selected from the group consisting of melanoma, pancreatic cancer, and colorectal cancer; or
(v) breast cancer, prostate cancer, renal cell carcinoma, bone metastasis, lung cancer or metastasis, osteosarcoma, multiple myeloma, astrocytoma, pilocytic astrocytoma, dysembryoplastic neuroepithelial tumor, oligodendrogliomas, ependymoma, glioblastoma multiforme, mixed gliomas, oligoastrocytomas, medulloblastoma, retinoblastoma, neuroblastoma, germinoma, teratoma, gangliogliomas, gangliocytoma, central gangliocytoma, primitive neuroectodermal tumors (PNET, e.g. medulloblastoma, medulloepithelioma, neuroblastoma, retinoblastoma, ependymoblastoma), tumors of the pineal parenchyma (e.g. pincocytoma, pineoblastoma), ependymal cell tumors, choroid plexus tumors, neuroepithelial tumors of uncertain origin (e.g. gliomatosis cerebri, astroblastoma), esophageal cancer, colorectal cancer, CNS, ovarian, melanoma pancreatic cancer, squamous cell carcinoma, hematologic cancer (e.g., leukemia, lymphoma, and multiple myeloma), colon cancer, rectum cancer, stomach cancer, kidney cancer, pancreas cancer, skin cancer, or a combination thereof.

71.-72. (canceled)


SEQUENCE LISTING (59 sequences)

SEQ ID NO: 1-40 - skipped (40 entries)

SEQ ID NO: 41 - protein, 9 aa, synthetic construct
  REGION 1..9: note = Synthetic: 1-A4
DGTLAVPFK

SEQ ID NO: 42-50 - skipped (9 entries)

SEQ ID NO: 51 - protein, 20 aa, synthetic construct
  REGION 1..20: note = Synthetic: peptide linker
  VARIANT 6..20: note = "GGGGS" may be present or absent
GGGGSGGGGSGGGGSGGGGS

SEQ ID NO: 52 - protein, 8 aa, synthetic construct
  REGION 1..8: note = Synthetic: peptide linker
GGGGGGGG

SEQ ID NO: 53 - protein, 6 aa, synthetic construct
  REGION 1..6: note = Synthetic: peptide linker
GGGGGG

SEQ ID NO: 54 - protein, 15 aa, synthetic construct
  REGION 1..15: note = Synthetic: peptide linker
EAAAKEAAAKEAAAK

SEQ ID NO: 55 - protein, 15 aa, synthetic construct
  REGION 1..15: note = Synthetic: peptide linker
  VARIANT 6..15: note = "EAAAK" may be present or absent
EAAAKEAAAKEAAAK

SEQ ID NO: 56 - protein, 46 aa, synthetic construct
  REGION 1..46: note = Synthetic: peptide linker
AEAAAKEAAAKEAAAKEAAAKALEAEAAAKEAAAKEAAAKEAAAKA

SEQ ID NO: 57 - protein, 5 aa, synthetic construct
  REGION 1..5: note = Synthetic: peptide linker
PAPAP

SEQ ID NO: 58 - protein, 12 aa, synthetic construct
  REGION 1..12: note = Synthetic: peptide linker
AEAAAKEAAAKA

SEQ ID NO: 59 - protein, 68 aa, synthetic construct
  REGION 1..68: note = Synthetic: peptide linker
  VARIANT 21..68: note = "AP" may be present or absent
APAPAPAPAPAPAPAPAPAPAPAPAPAPAPAPAPAPAPAPAPAPAPAPAPAPAPAPAPAPAPAPAPAP